(19) United States
(12) Patent Application Publication
(10) Pub. No.: US 2024/0383981 A1
(43) Pub. Date: Nov. 21, 2024

(54) USES OF T CELL ENGAGING PROTEINS IN THE TREATMENT OF CANCER

(71) Applicant: Boehringer Ingelheim International GmbH, Ingelheim am Rhein (DE)

(72) Inventors: Susanne HIPP, Biberach an der Riß (DE); Matus STUDENY, Bratislava (SK); Tillmann TAUBE, Biberach an der Riss (DE); Jin ZHANG, Riehen (CH)

(21) Appl. No.: 18/667,440

(22) Filed: May 17, 2024

(30) Foreign Application Priority Data
May 19, 2023 (EP) 23174355.0

(51) Int. Cl. C07K 16/28; A61K 39/00; A61P 35/00; C07K 16/30

(52) U.S. Cl. CPC C07K 16/2809; A61P 35/00; C07K 16/30; A61K 2039/507; A61K 2039/545; C07K 2317/31; C07K 2317/565

(57) ABSTRACT

The invention generally relates to the treatment of cancer by using DLL3/CD3 binding proteins. In particular, the present invention relates to uses and methods of using DLL3/CD3 binding proteins in a dosage and administration regimen for the treatment of cancer.

### SEQUENCE LISTING

The present disclosure contains a Sequence Listing which has been submitted electronically in XML format and is hereby incorporated by reference in its entirety. Said XML copy, created on May 17, 2024, is named PT24-019EP-US-SEQ26.xml and is 66,000 bytes in size.

### TECHNICAL FIELD

The invention generally relates to the treatment of cancer by using T cell engaging protein that specifically binds to CD3 and DLL3. In particular, the present invention relates to uses and methods of using DLL3/CD3 binding T cell engaging proteins in a dosage and administration regimen for the treatment of cancer.

### BACKGROUND INFORMATION

### DLL3

Delta-like ligand 3 (DLL3), also known as delta-like protein 3, is a developmental protein which plays a key role in somitogenesis during embryonic development (Geffers et al., J Cell Biol; 2007; 178(3); 465-476). The protein sequence of human DLL3 can be retrieved, for example, from the UniProt database (Uniprot: Q9NYJ7). DLL3 is a member of the Notch signaling pathway, more specifically a Notch ligand.

It is a well-suited target for cancer therapies. It is expressed in cancers of neuroendocrine origin and glioma but has very restricted tissue expression in healthy adult tissues.

Therapeutic proteins disclosed herein as well as in the applicant's earlier patent application WO 2019/234220 (herewith incorporated by reference) are humanized IgG-like T cell engagers (TcE) and are designed to induce T cell redirected lysis of cells that carry DLL3 (Delta-like 3) on the cell surface. This concept combines the selectivity of monoclonal antibodies with the therapeutic potential of cytolytic T cells.

With cancers known to express DLL3 molecule (also referred to herein as “DLL3 positive cancers”), the above-mentioned T cell engagers have the potential to provide benefit to these patients.

### Overview of Cancers Known to Express DLL3

DLL3 is known to be expressed in neoplasms of neuroendocrine origin (e.g., Neuroendocrine Neoplasms (NENs)), and furthermore in Merkel Cell Carcinomas (MCC), medullary thyroidal carcinoma, glioma and glioblastoma (Saunders et al., ACCR; 2017, Cancer Res; 2017; 77(13)(Suppl).

Neuroendocrine neoplasms (NENs) include neuroendocrine carcinomas (NECs), that are characterized by a poorly differentiated cytology and high proliferation rates, which result in an exceptionally aggressive clinical behavior (Nicholson et al., J Thorac Oncol; 2021; 17(3); 362-387). Thus, they are distinct from well-differentiated and more indolent group of neuroendocrine tumors (NETs). (FIG. 1). The NECs can be further subdivided into small-cell and large-cell variants and can arise from a variety of tissues. Most commonly, NECs originate from the lung, but are also known to originate in the gastroenteropancreatic system, the genitourinary system, skin and other tissues. In addition, a significant proportion of NECs have no identifiable primary site (Dasari et al., Cancer; 2018; 124(4); 807-815). It is thus recognized that the NECs represent a heterogenous group of tumors. The incidence rate of NECs overall is reported to be around 9.36 per 100,000. Small cell lung cancer (SCLC) is both the most common pulmonary NEC and most common NEC overall, accounting for around 13% of all lung cancer cases (Wang et al., Semin Cancer Biol; 2022; 86(Part 2); 543-554, Dasari et al., Cancer; 2018; 124(4); 807-815). A rare variant of pulmonary NEC is large-cell carcinoma of the lung (LCNEC of the lung).

NEC of any tissue of origin other than the lung are categorized as extrapulmonary NEC (epNEC). For this group of cancers, it is challenging to estimate trends regarding incidence due to the heterogeneity of the disease the associated lack of reliability in reporting, although the incidence of gastroenteropancreatic NECs (GEP-NECs) was reported to be stable in recent decades (Dasari et al., Cancer; 2018; 124(4); 807-815).

Diagnosis of NECs is based on distinct morphological characteristics of the tumor cells and expression of specific neuroendocrine markers via immunohistochemistry (Kawasaki et al., Nat Rev Clin Oncol; 2023; 20; 16-32). The tumors are further characterized based on site of the primary tumor (see above). In general, more men are affected by NEC and patients tend to be older (median age 65-67 years) (Dasari et al., Cancer; 2018; 124(4); 807-815, DiBonaventura., Ther Clin Risk Manage; 2019; 15; 355-366). Currently, there is no procedure available that is effective in early detection of SCLC and a large screening trial using computed tomography in high-risk groups has failed to shift diagnosis to earlier stages with a better prognosis (Silva et al., J Thorac Oncol; 2015; 11(2); 187-193). For epNEC, there are no disease-specific screening programs.

Treatment Options for NEC, Including SCLC and epNEC

Over the last several years, the backbone of first-line standard chemotherapy (platinum-etoposide) for patients with advanced SCLC (extensive-stage) has not changed, although the addition of checkpoint inhibitors to chemotherapy has recently been approved (Horn et al., N Engl J Med; 2018; 379(23); 2220-2229, Paz-Ares et al., Lancet; 2019; 394; 1929-1939). For the significant proportion of patients with brain metastases, radiotherapy of the brain may be used in addition to systemic therapy. Radiotherapy of other sites can be performed in case of symptomatic lesions. Patients failing initial therapy may receive further treatment, e.g., repeating first-line chemotherapy in case of a prolonged response (National Comprehensive Cancer Network. NCCN clinical practice guidelines in oncology (NCCN guidelines): small cell lung cancer (version 3.2023, Dec. 21, 2022), Dingemans et al., Ann Oncol; 2021; 32(7); 839-853). For patients with SCLC that are refractory to initial treatment or experience early relapse, but are eligible for second-line therapy, a variety of chemotherapy options are available, including topotecan and lurbinectedin. For the third-line treatment setting and beyond, there is no approved and accepted standard of care. The minority of patients presenting with early-/limited-stage disease may be eligible for surgery in select cases, but for most patients, systemic therapy is indicated, with or without the addition of radiotherapy.

Principles of therapy for epNEC and LCNEC of the lung are similar to SCLC (National Comprehensive Cancer Network. NCCN Clinical Practice Guidelines in Oncology (NCCN Guidelines): neuroendocrine and adrenal tumors (version 1.2022—May 23, 2022)). For the majority of patients, who present with advanced stages of disease, systemic therapy is the preferred option, with or without addition of radiotherapy.

Platinum-based chemotherapy is the standard of care in the frontline setting. Less commonly, patients may receive other regimens such as FOLFOX, FOLFIRI or FOLFIRINOX, temozolomide and capecitabine or pembrolizumab (only in case of high tumor mutational burden or mismatch-repair deficiency). Addition of immunotherapy to chemotherapy currently does not represent an approved treatment option for patients with epNEC. There is no consensus or strong recommendation for treatment in the relapsed/refractory setting of epNECs, due to limited efficacy data. In eligible patients with resectable or locally advanced disease, the same chemotherapy regimens as in metastatic disease are recommended and can be combined with surgery and/or radiotherapy.

The clinical course of SCLC and epNECs is highly aggressive, and the disease usually progresses very rapidly (Pietanza et al., Clin Cancer Res; 2015; 21(10); 2244-2255). While these tumors are generally sensitive to chemotherapy and radiotherapy initially, they will usually relapse quickly. Treatment outcomes have improved only modestly in the past years, and remain comparatively poor, especially in the setting of advanced disease (Bogart et al., J Clin Oncol; 2022; 40(6); 661-671). In the setting of extensive-stage (ES)-SCLC, the addition of checkpoint inhibitors to chemotherapy has improved median overall survival (mOS) only by ˜2 months, and overall, it continues to remain poor at ˜13 months (Horn et al., N Engl J Med; 2018; 379(23); 2220-2229, Paz-Ares et al., Lancet; 2019; 394; 1929-1939). The prognosis for patients with advanced or metastatic epNEC is similarly dismal, with platinum-based chemotherapy leading to mOS of ˜11-15 months in patients with advanced digestive NEC (Morizane et al., JAMA Oncol; 2022; 8(10); 1447-1455). For NEC patients, in particular epNEC patients with advanced disease experiencing relapse, which almost invariably occurs, outcomes are even worse, e.g., mOS of 7-9 months for patients with relapsed/refractory SCLC receiving treatment (Trigo et al., Lancet Oncol; 2020; 21; 645-654, Baize et al., Lancet Oncol; 2020; 21; 1224-1233). Survival times in later-line therapy decrease even further and many patients are ineligible for multiple lines of treatment due to rapidly declining performance status and toxicity of standard therapy, which is chemotherapy-based.

To summarize, while often advanced/metastatic SCLC and epNECs will initially respond to established treatment, the patients will almost invariably experience relapse and eventually succumb to their cancer. For this reason, this group of cancers represents a major challenge for the physicians treating and the unsatisfactory outcomes with current Standard of Care (SoC) treatment indicate a high need for improvement.

As disclosed below, and as evidenced by the Examples, the present inventors provide a solution to this challenge.

### BRIEF SUMMARY OF THE INVENTION

The present invention is based on a treatment using a dosing regimen of a T cell engaging protein that specifically binds to CD3 and DLL3 for a cancer, said cancer preferably being selected from (i) Neuroendocrine Neoplasms (NEN) including Small cell lung cancer (SCLC), Large Cell neuroendocrinecarcinoma of the lung (LCNEC), extrapulmonary neuroendocrine carcinoma (epNEC), neuroendocrine tumors (NET), as well as (ii) Merkel Cell Carcinomas (MCC), medullary thyroidal carcinoma, glioma and glioblastoma. Due to the dosing regimen, treatment side effects, such as a cytokine release syndrome (CRS), can be reduced to a degree allowing for unprecedented high doses of the T cell engaging protein during the treatment without considerable safety concerns caused by the treatment.

In a first aspect, the present invention provides a T-cell engaging protein comprising a first antigen binding unit that specifically binds to DLL3 and a second antigen binding unit that specifically binds to CD3 for use in a method of treatment of a DLL3-positive cancer or a cancer selected from a Neuroendocrine Neoplasm (NEN), Merkel cell carcinoma (MCC), medullary thyroidal carcinoma and glioma, the method comprising a step-in cycle, said step-in cycle comprising the steps of:
administering to a subject an initial dose of said T-cell engaging protein, said initial dose ranging from about 5 μg/kg of the subject's body weight to about 40 μg/kg of the subject's body weight,
administering to the subject an elevated dose, which is elevated as compared to said initial dose, of said T-cell engaging protein, the elevated dose ranging from about 15 μg/kg of the subject's body weight to about 100 μg/kg of the subject's body weight, or the elevated dose ranging from about 5 mg to about 20 mg, and
administering to the subject a further dose of the T-cell engaging protein, said further dose ranging from about 80 μg/kg of the subject's body weight to about 1600 μg/kg of the subject's bodyweight, wherein said further dose is equal to or higher than said elevated dose, or said further dose is ranging from about 5 mg to about 70 mg.

In a second aspect, the present invention provides a T-cell engaging protein comprising a first antigen binding unit that specifically binds to DLL3 and a second antigen binding unit that specifically binds to CD3 for use in a method of treatment of a DLL3-positive cancer or a cancer selected from a Neuroendocrine Neoplasm (NEN), Merkel cell carcinoma (MCC), medullary thyroidal carcinoma and glioma, the method comprising a step-in cycle, said step-in cycle comprising the steps of:
administering to a subject an initial dose of said T-cell engaging protein, said initial dose ranging from about 5 μg/kg of the subject's body weight to about 15 μg/kg of the subject's body weight,
administering to the subject an elevated dose, which is elevated as compared to said initial dose, of said T-cell engaging protein, the elevated dose ranging from about 15 μg/kg of the subject's body weight to about 100 μg/kg of the subject's body weight, or the elevated dose ranging from about 5 mg to about 20 mg, and
administering to the subject a further dose of the T-cell engaging protein, said further dose ranging from about 5 mg to about 70 mg.

In a third aspect, the present invention provides a T-cell engaging protein comprising a first antigen binding unit specifically binding to DLL3 and a second antigen binding unit specifically binding to CD3 for use in a method of treatment of a DLL3-positive cancer or a cancer selected from a Neuroendocrine Neoplasm (NEN), Merkel cell carcinoma (MCC), medullary thyroidal carcinoma and glioma;
the method comprising a step-in cycle, said step-in cycle comprising the steps of:
   administering to a subject an initial dose of said T-cell engaging protein, said initial dose ranging from about 20 μg/kg of the subject's body weight to about 40 μg/kg of the subject's body weight,
   administering to the subject an elevated dose, which is elevated as compared to said initial dose, of said T-cell engaging protein, the elevated dose ranging from about 60 μg/kg of the subject's body weight to about 100 μg/kg of the subject's body weight,
   administering to the subject a further dose of the T-cell engaging protein, said further dose ranging from about 80 μg/kg of the subject's body weight to about 1600 μg/kg of the subject's bodyweight, wherein said further dose is equal to or higher than said elevated dose.

Of note, said initial dose may be yet lower, be it in the context of other treatment regimens disclosed herein or in the course of the regimen defined above. As such, the lower limit of said initial dose may be about 5 μg/kg (and the upper limit about 40 μg/kg as defined above), preferred values including said about 5 μg/kg as well as about 10 μg/kg. Preferably, the initial dose is about 10 μg/kg or about 30 μg/kg.

Similarly, also said elevated dose may be lower than specified above, also in the context of other treatment regimens disclosed herein or in the course of the regimen defined above. As such, the lower limit of said elevated dose may be about 15 μg/kg (and the upper limit about 100 μg/kg as defined above), preferred values including said about 15 μg/kg as well as about 30 μg/kg, provided that said elevated dose is higher as compared to said initial dose. Additionally, the elevated dose may range from about 5 mg to about 20 mg.

Preferably, the elevated dose is about 30 μg/kg, about 90 μg/kg, or about 10 mg.

In some embodiments, said further dose is equal to a target dose, said target dose being the dose to be administered during a first treatment cycle which optionally directly follows said step-in cycle.

In some embodiments, said initial dose, said elevated dose and/or said further dose is/are administered once; or said initial dose, said elevated dose and/or said further dose is/are administered more than once, optionally 2 to 10 times, wherein optionally the number of administrations is determined based on treatment tolerability including presence and/or degree of a cytokine release syndrome (CRS) and/or of an immune effector cell-associated neurotoxicity syndrome (ICANS), and wherein further optionally said elevated dose follows the initial dose only if said CRS, to the extent determined, is rated grade 1 or less in terms of NCI CTC grade and/or if said ICANS, to the extent determined, is rated grade 1 or less, and/or wherein further optionally said further dose follows the elevated dose only if said CRS, to the extent determined, is rated grade 1 or less in terms of NCI CTC grade and/or if said ICANS, to the extent determined, is rated grade 1 or less.

In some embodiments, said initial dose is administered three times to ten times within the step-in cycle.

In some embodiments, said initial dose is administered three times within three weeks, optionally on day 1, day 8 and day 15 of the three weeks.

More generally speaking, preference is given to weekly administration. As such, in case of an extended step-in cycle (preferably in response to insufficient treatment tolerability) comprising, say, 5 doses in total, said step-in cycle will last five weeks, with administrations to be effected on days 1, 8, 15, 22 and 29, wherein certain deviations from said specific days are acceptable as detailed further below.

In some embodiments, said step-in cycle has a duration of at least three weeks.

In some embodiments, said initial, elevated and further doses are administered within three weeks, said initial dose being administered once, wherein optionally said initial dose is administered on day 1, said elevated dose is administered on day 8 and said further dose is administered on day 15 of said step-in cycle.

In some embodiments, said initial dose ranges from about 25 μg/kg of the subject's body weight to about 35 μg/kg of the subject's body weight, and optionally said initial dose is 30 μg/kg of the subject's body weight; said elevated dose ranges from about 85 μg/kg of the subject's body weight to about 95 μg/kg of the subject's body weight, and optionally said elevated dose is about 90 μg/kg of the subject's body weight; and said further dose ranges from about 85 μg/kg of the subject's body weight to about 1550 μg/kg of the subject's body weight, and optionally the further dose is about 90 μg/kg, about 180 μg/kg, about 270 μg/kg, about 360 μg/kg, about 540 μg/kg, about 720 μg/kg, about 1080 μg/kg or about 1530 μg/kg of the subject's body weight.

Generally speaking, said step-in cycle will be performed once for a given patient. Yet, it is conceivable that a given patient has to interrupt therapy in accordance with the invention for reasons which are not related to said therapy or potential side effects thereof. This may be the case when the patient has to undergo surgery (either related to the malignancy to be treated in accordance with the invention or for other reasons). After such interruption, the treatment will likely have to be started all over again and in that sense, said step-in cycle would indeed be repeated with the same patient.

In some embodiments, the method further comprises a first treatment cycle following the step-in cycle, said first treatment cycle comprising the steps of:
administering to a subject a first target dose of said T-cell engaging protein, said first target dose ranging from about 80 μg/kg of the subject's body weight to about 1600 μg/kg of the subject's body weight,
administering to the subject a second target dose of said T-cell engaging protein, said second dose ranging from about 80 μg/kg of the subject's body weight to about 1600 μg/kg of the subject's body weight, and
administering to the subject a third target dose of said T-cell engaging protein, said third dose ranging from about 80 μg/kg of the subject's body weight to about 1600 μg/kg of the subject's bodyweight,
   wherein optionally the amount of all three target doses is identical.

In some embodiments, the method further comprises a first treatment cycle following said step-in cycle, said first treatment cycle comprising the steps of:
administering to a subject a first target dose of the T-cell engaging protein, said first target dose ranging from about 5 mg to about 70 mg,
administering to the subject a second dose of the T-cell engaging protein, said second dose ranging from about 5 mg to about 70 mg, and
administering to the subject a third dose of the T-cell engaging protein, said third dose ranging from about 5 mg to about 70 mg,
   wherein optionally the amount of all three target doses is identical.

In some embodiments, at least one of said first, second or third target doses of said first treatment cycle comprises more than 30 mg.

In some embodiments, at least one of said first, second or third target doses of the first treatment cycle is about 5 mg, about 10 mg, about 20 mg, about 30 mg, about 40 mg, about 50 mg, about 60 mg, or about 70 mg, optionally about 10 mg or about 60 mg.

In preferred embodiments, said first treatment cycle is not repeated or said first treatment cycle is repeated at least once.

Such repetitions are generally preferred or medically indicated to secure successful treatment. As such, envisaged is a continuation of said first treatment cycle for up to three years or, where necessary more.

Accordingly, said first treatment cycle may be repeated 2, 3, 4, 5, 6, 7, 8, 9, 10, 20, 30, 40, 50, 60, 70, 80, 90, 100, 150 times or last for 6 months, 12 months, 18 months, 24 months, 30 months or 36 months. Repetitions or extended repetitions of said first treatment cycle are preferred in those embodiments where no second treatment cycle(s) are applied.

In preferred embodiments, said first, second and third target doses are administered within three weeks of said first treatment cycle, wherein optionally said first target dose is administered on day 1, the second target dose is administered on day 8 and the third target dose is administered on day 15 of said first treatment cycle, wherein optionally said first treatment cycle has a duration of three weeks.

In preferred embodiments, said first treatment cycle is followed by a second treatment cycle, said second treatment cycle comprising the step of or consisting of the step:
administering to the subject a target dose of said T-cell engaging protein, said target dose ranging from about 80 μg/kg of the subject's body weight to about 1600 μg/kg of the subject's body weight; wherein said second treatment cycle is optionally repeated at least once.

In preferred embodiments, said first treatment cycle is followed by a second treatment cycle, said second treatment cycle comprising the step of or consisting of the step:
administering to the subject a target dose of the T-cell engaging protein, said target dose ranging from about 5 mg to about 70 mg;
   wherein said second treatment cycle is optionally repeated at least once.

Such repetitions are generally preferred or medically indicated to secure successful treatment. As such, envisaged is a continuation of said second treatment cycle for up to three years or, where necessary more.

Accordingly, said second treatment cycle may be repeated 2, 3, 4, 5, 6, 7, 8, 9, 10, 20, 30, 40, 50, 60, 70, 80, 90, 100, 150 times or last for 6 months, 12 months, 18 months, 24 months, 30 months or 36 months.

More preferred target doses are between about 5 mg and about 60 mg, including about 5, about 10, about 20, about 30, about 40, about 50 and about 60 mg, about 5, about 10, about 30 and about 60 mg being especially preferred. It is generally to be understood that any specific values recited may define a lower or upper limit of a sub-interval in addition to the sub-interval from about 5 mg to about 60 mg explicitly spelled out above.

In preferred embodiments, said second treatment cycle has a total duration of three weeks and/or said target dose is administered on day 1 of said three weeks.

In preferred embodiments, said first treatment cycle directly follows said step-in cycle; and/or said second treatment cycle directly follows said first treatment cycle.

Cancers amenable to treatment and not belonging to NENs include glioma (such as local glioma, diffuse glioma, multiple glioma and glioblastoma), medullary thyroidal carcinoma, and Merkel cell carcinoma.

Glioma also may include astrocytoma, especially IDH mutant, CNS WHO Grade 2-4; oligodendroglioma, especially IDH mutant and 1p19q co-deleted, CNS WHO Grade 2 and 3; and/or glioblastoma (IDH wild type) according to the WHO 2021 brain tumour classification.

NEN in turn can be subdivided into Neuroendocrine Carcinomas (NECs) and Neuroendocrine Tumors (NETs). NECs include Lung Neuroendocrine Carcinoma preferably selected from the group consisting of Lung-NEC such as LCNEC of the lung, Extrapulmonary (ep) NEC including NEC with unknown primary site, small cell lung cancer (SCLC) including extensive-stage (ES)-SCLC, and limited-stage (LS) SCLC; or said NEN is a Neuroendocrine Tumor (NET) such as grade 3 NET.

All of the above listed cancers are considered to be known to express DLL3. In other words, said cancers are considered to be inherently characterized as being DLL3-positive and/or expressing DLL3. As such, a separate determination of whether said cancers are DLL3-positive prior to administration of the treatment in accordance with the invention is not a requirement.

In some embodiments, a premedication comprising or consisting of a non-steroidal anti-inflammatory drug (NSAID), such as acetaminophen, an anti-histaminicum and/or a steroid, preferably a corticosteroid such as prednisolone or dexamethasone, is to be administered prior to at least one of the doses, optionally prior to each dose. “Dose” in this context refers to any dose, be it during the step-in cycle or any of the treatment cycles.

More preferably, a premedication comprising or consisting of a non-steroidal anti-inflammatory drug, an anti-histaminicum and a steroid is given during said step-in cycle.

Depending on the occurrence of adverse effects or limited tolerability in response to the administration of said T-cell engaging protein, premedication may be adjusted. This applies in particular to said steroid. As such, if it is observed that a given dose of said T-cell engaging protein is well tolerated (criteria therefore are disclosed herein), doses of said premedication may be reduced as compared to the previous dose of said premedication. In particular, the dose of said steroid to be administered prior to a dose of said T-cell engaging protein is reduced, such as to about 10% to about 90% of the previous dose including to about ⅔ or about ⅓, preferably about to about 50% of said previous dose, or skipped altogether when satisfactory tolerance of the T-cell engaging protein is observed upon the preceding administration of said T-cell engaging protein.

Such reductions may be performed at any time, be it during said step-in cycle or the treatment cycles. A preferred point in time is at the beginning of the first treatment cycle.

As regards the non-steroidal anti-inflammatory drug and the anti-histaminicum, it is preferred to continue administration of the same amounts.

Any further adaptations of the premedication can be performed by the attending clinician without further ado.

In some embodiments, the subject has been treated with a chemotherapy involving administration of e.g., cytostatic agents or inhibitors of cell growth and/or division prior to the step-in cycle. This includes a scenario wherein said chemotherapy has been administered as a first line treatment and has failed, as explained in the background section herein above as well as in the section entitled “Indications/patient population”. In certain embodiments, said subject has alternatively or in addition undergone a therapy selected from radiotherapy and surgery.

Having said that, and this is subject of a separate embodiment further below, the T cell engaging proteins of the invention may also be part of a combination therapy, one or more further agent(s) to be given in the course of said combination therapy being chemotherapy or, equivalently, one or more chemotherapeutic agent(s).

In either case, preferred chemotherapeutic agents are platinum-based chemotherapeutic agents such as carboplatin; cisplatin; etoposide; temozolomide; and topotecan; and furthermore lurbinectedin and capecitabine. Said chemotherapy in turn may be a combination of a plurality of agents such a platinum-based agent (e.g., carboplatin) with another cytostatic agent (e.g., etoposide). The use of combination therapy (T cell engager of the invention plus one or more chemotherapeutic agents) is not particularly limited to any of the medical indications disclosed herein. To the extent NENs (such as SCLC, epNEC and LCNEC of the lung), NETs as well as Merkel cell carcinoma are considered, combinations with one, more or all of platin-based chemotherapy, etoposide and topotecan with T cell engagers is preferred. For the treatment of LS-SCLC radiotherapy and/or surgery can be used in combination with the T cell engagers of the invention. For the treatment of glioma and glioblastoma, combinations of said T cell engagers with temozolomide are preferred. Yet more specific information about preferred combination therapies can be found in the section entitled “Indications/patient populations” further below.

Further in terms of combination therapies, in addition to a T cell engaging molecule of the invention, and optionally in addition to said chemotherapeutic agent, an immune-checkpoint inhibitor such as an anti-PD-1 antibody or an anti-PD-L1 antibody is to be administered. Preferred anti-PD-1 antibodies are ezabenlimab, pembrolizumab, nivolumab, or pidilizumab. Preferred anti-PD-L1 antibodies are durvalumab, avelumab or atezolizumab.

In some embodiments, an occurrence of cytokine release syndrome is to be treated with an anti-cytokine release syndrome agent, preferably selected from the group consisting of a steroid such as a corticosteroid; an IL-6 antagonist and an IL-6R antagonist. A preferred IL-6R antagonist is tocilizumab. Preferred corticosteroids are those disclosed above in the context of premedication.

In some embodiments, said T-cell engaging protein comprises immunoglobulin (Ig) domains, preferably 12 Ig domains.

The term “immunoglobulin domain” has its art-established meaning. It refers to a protein domain that consists of a two-layer sandwich of seven to nine antiparallel beta-strands arranged in two beta-sheets with a Greek key topology. Further information about Ig domains can be found in entry PF00047 of the Pfam database (Pfam: The protein families database in 2021: J. Mistry, S. Chuguransky, L. Williams, M. Qureshi, G. A. Salazar, E. L. L. Sonnhammer, S. C. E. Tosatto, L. Paladin, S. Raj, L. J. Richardson, R. D. Finn, A. Bateman Nucleic Acids Research (2020) doi: 10.1093/nar/gkaa913).

In a further embodiment, said Ig domains are comprised in two polypeptide chains, wherein each polypeptide chain comprises six Ig domains.

In a further embodiment, either antigen binding unit comprises six complementarity determining regions (CDRs).

In a further embodiment, said T-cell engaging protein comprises a first antigen binding unit specifically binding to DLL3, comprising light chain CDRs comprising the amino acid sequences of SEQ ID NO:13 (CDR1), SEQ ID NO:14 (CDR2) and SEQ ID NO:15 (CDR3) and heavy chain CDRs comprising the amino acid sequences of SEQ ID NO:16 (CDR1), SEQ ID NO:17 (CDR2) and SEQ ID NO:18 (CDR3) and a second antigen binding unit specifically binding to CD3 comprising light chain CDRs comprising the amino acid sequences of SEQ ID NO:55 (CDR1), SEQ ID NO:56 (CDR2) and SEQ ID NO:57 (CDR3) and heavy chain CDRs comprising the amino acid sequences of SEQ ID NO:58 (CDR1), SEQ ID NO:59 (CDR2) and SEQ ID NO:60 (CDR3).

In a further embodiment, said T-cell engaging protein comprises (i) a first antigen binding unit specifically binding to DLL3 comprising a light chain variable domain comprising the amino acid sequence of SEQ ID NO:41 and heavy chain variable domain comprising the amino acid sequence of SEQ ID NO:42 and (ii) a second antigen binding unit specifically binding to CD3 comprising a light chain variable domain comprising the amino acid sequence of SEQ ID NO:67 and a heavy chain variable domain comprising the amino acid sequence of SEQ ID NO:68.

In a further embodiment, said T-cell engaging protein comprises a first single chain Fab forming a first antigen binding unit comprising the sequence of SEQ ID NO:51 and a second single chain Fab comprising the sequence of SEQ ID NO:71 forming a second antigen binding unit.

In a further embodiment, said T-cell engaging protein comprises or consists of a first polypeptide chain specific for DLL3 comprising an amino acid sequence of SEQ ID NO:75 and a second polypeptide chain specific for CD3 comprising the amino acid sequence of SEQ ID NO:79. To the extent said T-cell engaging protein consists of said first and second polypeptide chains, said T-cell engaging protein is bispecific.

In some embodiments, no or essentially no cytokine release syndrome occurs after said elevated dose of said step-in cycle in the subject. This would be a scenario where premedication may be adjusted as explained above. Furthermore, this would generally allow for a further dose in the course of said step-in cycle which is identical to the target dose to be administered in the ensuing first treatment cycle.

In some embodiments, said T-cell engaging protein is the only anti-cancer agent to be administered.

In some embodiments, at least one further anti-cancer agent is to be administered, said further anti-cancer agent preferably being a chemotherapeutic agent, preferred chemotherapeutic agents being those disclosed above.

In a fourth aspect, the invention provides a T-cell engaging protein comprising a first antigen binding unit specifically binding to DLL3 and a second antigen binding unit specifically binding to CD3 for use in a method of treatment of a DLL3-positive cancer or a cancer selected from a Neuroendocrine Neoplasm (NEN), Merkel cell carcinoma (MCC), medullary thyroidal carcinoma and glioma;
the method comprising a step-in cycle, the step-in cycle comprising the steps of:
   administering to the subject an initial dose, an elevated dose, which is elevated as compared to said initial dose, and a further dose of said T-cell engaging protein, wherein a ratio between said initial, elevated and further dose per kilogram of the subject's body weight is about 1:about 3:about 9.

In a fifth aspect, the invention provides a T-cell engaging protein comprising a first antigen binding unit specifically binding to DLL3 and a second antigen binding unit specifically binding to CD3 for use in a method of treatment of a DLL3-positive cancer or a cancer selected from a Neuroendocrine Neoplasm (NEN), Merkel cell carcinoma (MCC), medullary thyroidal carcinoma and glioma;
the method comprising a step-in cycle, the step-in cycle comprising the steps of:
   administering to a subject an initial dose of said T-cell engaging protein, said initial dose ranging from about 5 μg/kg of the subject's body weight to about 15 μg/kg of the subject's body weight,
   administering to the subject an elevated dose, which is elevated as compared to said initial dose, of said T-cell engaging protein, the elevated dose ranging from about 20 μg/kg of the subject's body weight to about 40 μg/kg of the subject's body weight,
   administering to the subject a further dose of said T-cell engaging protein, said further dose ranging from about 80 μg/kg of the subject's body weight to about 1600 μg/kg of the subject's bodyweight.

Embodiments of the first aspect of the invention define mutatis mutandis preferred embodiments of said second, third, fourth and fifth aspect.

Further aspects, embodiments, uses and methods involving the binding proteins of the invention will become clear from the following detailed description of the invention and from the appended claims.

### BRIEF DESCRIPTION OF THE DRAWINGS

FIG. 1: Classification of Neuroendocrine Neoplasms adapted from Rindi et al., Endocr Pathol; 2022; 33; 115-154.

FIG. 2: Phase Ia trial design.

FIG. 3: Safety Results from regimens A, B1 and B2 (data output Q1/2023): Frequency of patients with investigator defined treatment related AEs occurring with incidence in preferred term >=5% by highest CTCAE grade, on treatment period.

FIG. 4A: Frequency of patients with cytokine release syndrome by step-in phase, toxicity grade group in regimen B2 (data output Q1/2023).

FIGS. 4B1-B3: Frequency [N (%)] of patients with cytokine release syndrome by dose level and highest CTCAE grade—Treated set—Regimen A (data output Q1/2024).

FIG. 4C: Frequency [N (%)] of patients with cytokine release syndrome by dose level and highest CTCAE grade—Treated set—Regimen B1 (data output Q1/2024).

FIGS. 4D1-D3: Frequency [N (%)] of patients with cytokine release syndrome by treatment period, target dose and highest CTCAE grade—Treated set—Regimen B2 (data output Q1/2024).

FIGS. 4E1-E3: Frequency [N (%)] of patients with cytokine release syndrome by treatment period, target dose and highest CTCAE grade—Treated set—Regimen B3 (data output Q1/2024).

FIG. 4F: Frequency [N (%)] of patients with cytokine release syndrome by treatment period, highest CTCAE grade (grouped) on treatment period, Treated set—Regimen B2 and B3 (data output Q1/2024).

FIG. 4G: Frequency [N (%)] of patients with drug-related potential neurologic toxicity including ICANS (narrow) by user-defined categories by regimen, highest CTCAE grade (grouped), and preferred term on-treatment period treated set—Regimen B2 and B3 (data output Q1/2024).

FIG. 5A: Response in Regimen A by dose level (data output Q1/2023).

FIG. 5B: Response in Regimen B2 (data output Q1/2023).

FIGS. 5-C Best overall tumor response and disease control for regimens A, B1, B2, B3 (data output Q1/2024)

FIGS. 5D1-D3: Best overall tumor response and disease control by dose level—Efficacy Set—Regimen B2 (data output Q1/2024)

FIGS. 5E1-E2: Best overall tumor response and disease control by dose level—Efficacy Set—Regimen B3 (data output Q1/2024)

FIG. 5F: Best overall tumor response and disease control by tumor type—Efficacy Set—Regimen B2 (data output Q1/2024)

FIG. 5G: Best overall tumor response and disease control by tumor type—Efficacy Set—Regimen B3 (data output Q1/2024)

FIG. 5 H: Waterfall plot of best percentage change from baseline in target lesions by dose level—Efficacy Set—Regimen B2 (data output Q1/2024)

FIG. 6A: Cytokine levels in peripheral blood at different timepoints after administration of BI 764532 in a Q3W regimen (Regimen A). Each line represents the mean of the patients as indicated at the bottom of the Figure. Arrows at the top of the figure indicated timing of BI 764532 administration (data output Q1/2023).

FIG. 6B: Cytokine levels in peripheral blood at different timepoints after administration of BI 764532 in a QW regimen (Regimen B1). Each line represents the mean of the number of patients as indicated at the bottom of the Figure. Arrows at the top of the figure indicated timing of BI 764532 administration (data output Q1/2023).

FIG. 6C: Cytokine levels in peripheral blood at different timepoints after administration of BI 764532 in a QW regimen with a first dose of 30 μg/kg, a second dose of 90 μg/kg, and a target dose of either 90, 180, 270, 360, or 540 μg/kg (Regimen B2). Each line represents the mean of the number of patients as indicated at the bottom of the Figure. Arrows at the top of the figure indicated timing of BI 764532 administration (data output Q1/2023).

FIG. 6D: Cytokine levels in peripheral blood at different timepoints after administration of BI 764532 in a QW regimen with a first dose of 30 μg/kg, a second dose of 90 μg/kg, and a target dose of either 90, 180, 270, 360, 540, 720, 1080, or 1530 μg/kg (Regimen B2). Each line represents the mean of the number of patients as indicated at the bottom of the Figure. Arrows at the top of the figure indicated timing of BI 764532 administration (data output Q1/2024).

FIG. 6E: Cytokine levels in peripheral blood at different timepoints after administration of BI 764532 in a QW regimen with a first dose of 10 μg/kg, a second dose of 30 μg/kg, a third dose of 90 μg/kg, and a target dose of either 90, 180, 270, 360, 540, 720, or 1080 μg/kg (Regimen B3). Each line represents the mean of the number of patients as indicated at the bottom of the Figure. Arrows at the top of the figure indicated timing of BI 764532 administration (data output Q1/2024).

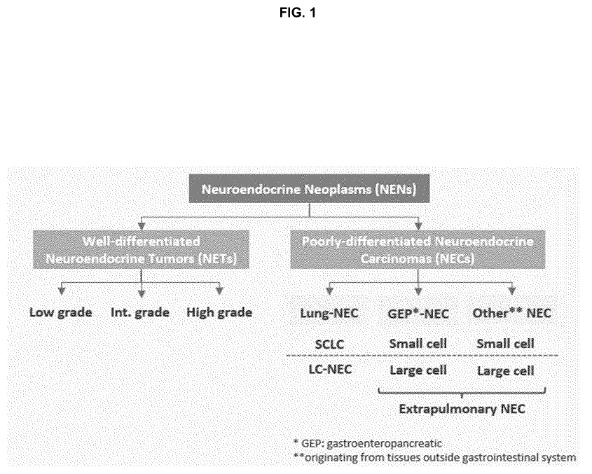

### DETAILED DESCRIPTION OF THE INVENTION

### Used Terms and Definitions

The above and other aspects and embodiments of the invention will become clear from the further description herein.

Any definition given below serves at least two purposes: establish the meaning of terms used in this disclosure and provide a description of preferred or exemplary embodiments.

Unless indicated or defined otherwise, all terms used have their usual meaning in the art, which will be clear to the skilled person. Reference is for example made to the standard handbooks, such as Sambrook et al, “Molecular Cloning: A Laboratory Manual” (2nd Ed.), Vols. 1-3, Cold Spring Harbor Laboratory Press (1989); Lewin, “Genes IV”, Oxford University Press, New York, (1990), and Roitt et al, “Immunology” (2nd Ed.), Gower Medical Publishing, London, New York (1989), as well as to the general background art cited herein. Furthermore, unless indicated otherwise, all methods, steps, techniques and manipulations that are not specifically described in detail can be performed and have been performed in a manner known per se, as will be clear to the skilled person. Reference is for example again made to the standard handbooks, to the general background art referred to above and to the further references cited therein.

When used herein the term “comprising” and variations thereof such as “comprises” and “comprise” can be substituted with the term “containing” or “including” or “having.”

The T cell engagers of the present invention are generally referred to as “T-cell engaging proteins” or, with equivalent meaning, “T-cell engaging molecules”, or “TCEs”. The term “protein” has its art-established meaning and refers to a biomolecule comprising or consisting of one or a plurality of polypeptide chains, wherein in case of plurality of polypeptide chains, said chains are bound to each other non-covalently or covalently such as via one or more disulfide bonds. A “polypeptide” or “polypeptide chain” is a polycondensate of amino acids, preferably of the naturally occurring proteinogenic amino acids. Preferred polypeptide sequences comprised in T-cell engaging proteins of the invention are reproduced further below as well as in the attached sequence listing.

Of note, while the present disclosure refers to TCEs which are also described in WO 2019/234220, the dosage regimens of the present invention are not so limited. As explained in the section “Contributions made by the present invention” further below, the present invention solves the more general problem of adverse effects occurring in response to TCEs. In other words, the notion of “TCEs of the invention” or “TCEs in accordance with the invention” indeed applies to any TCE, be it explicitly disclosed herein or embraced by the functional definition “TCE”, more specifically by the term “T-cell engaging protein comprising a first antigen binding unit specifically binding to DLL3 and a second antigen binding unit specifically binding to CD3”.

In addition to the TCEs disclosed further below, the term “TCE” embraces Tarlatamab and TCEs described in WO21200898 A1, WO19131988 A1, WO22237647 A1, WO20069028 A1, and WO22256500 A2.

TCEs to be used in accordance with the invention comprise the two binding units defined in the first and other aspects of the invention. As such, further binding units may be, but not have to be present. In other words, the TCEs may be tri-specific or multi-specific, while preference is given to bispecific TCEs which are subject of preferred embodiments or disclosed in WO 2019/234220.

Said T-cell engaging proteins, in particular said bispecific TCEs, preferably share structural and functional features with antibodies, more specifically Immunoglobulins of the G type (IgGs). Such shared features preferably include the presence of complementarity determining regions (CDRs) conferring specificity as well as the molecular architecture made of immunoglobulin domains. This is why sometimes in the context of characterizing said bispecific T-cell engaging proteins, terminology from the field of antibodies is used.

Having said that, at least certain preferred bispecific T-cell engaging proteins are not antibodies in a strict sense. Key differences include the bi-specificity as well as linkers (as defined further below) connecting the constant domain of a given light chain with the variable domain of the corresponding heavy chain (“corresponding” in this context meaning that the two mentioned chains comprise in particular antigen binding unit). As a consequence, preferred molecular architectures of said bispecific T-cell engaging proteins comprise or consist of two polypeptide chains (as opposed to four polypeptide chains generally present in an IgG molecule).

The term “sequence” as used herein (for example in terms like “heavy/light chain sequence”, “antibody sequence”, “variable domain sequence”, “constant domain sequence” or “protein sequence”), should generally be understood to include both the relevant amino acid sequence as well as nucleic acid sequences or nucleotide sequences encoding the same, unless the context requires a more limited interpretation.

An “antigen binding unit” as used herein refers to a polypeptide capable of binding to its specific target or antigen and comprising the minimal structural requirements derived from an antibody (typically present in an antibody) which allow for target binding. Thus, an antigen binding unit comprises at least the presence of three light chain and three heavy chain CDR sequences, preferably at least a light chain variable domain and a heavy chain variable domain.

The generalized structure of an antibody or immunoglobulin is well known to those of skill in the art. These molecules are heterotetrameric glycoproteins, typically of about 150,000 daltons, composed of two identical light (L) chains and two identical heavy (H) chains and are typically referred to as full length antibodies.

Each light chain is covalently linked to a heavy chain by one disulfide bond to form a heterodimer, and the heterotetrameric molecule is formed through a covalent disulfide linkage between the two identical heavy chains of the heterodimers. Although the light and heavy chains are linked together by one disulfide bond, the number of disulfide linkages between the two heavy chains varies by immunoglobulin isotype. Each heavy and light chain also has regularly spaced intrachain disulfide bridges. Each heavy chain has at the N-terminus a variable domain (VH), followed by three or four (in case of IgE) constant domains (CH1, CH2, CH3, and CH4), as well as a hinge region between CH1 and CH2. Each light chain has two domains, an N-terminal variable domain (VL) and a C-terminal constant domain (CL). The VL domain associates non-covalently with the VH domain, whereas the CL domain is commonly covalently linked to the CH1 domain via a disulfide bond. Particular amino acid residues are believed to form an interface between the light and heavy chain variable domains (Chothia et al, 1985, J. Mol. Biol. 186:651-663). Variable domains are also referred to herein as variable regions or Fv and denote the part that confers specificity to an antibody for the antigen by carrying the antigen-binding site.

The “light chain variable domain” (or “light chain variable region”) and “heavy chain variable domain” (or “heavy chain variable region”) as used herein have the same general structure and each domain essentially consists of four framework (FR) regions whose sequences are widely conserved, which are referred to in the art and hereinbelow as “framework region 1” or “FR1”; as “framework region 2” or “FR2”; as “framework region 3” or “FR3”; and as “framework region 4” or “FR4”, respectively; which framework regions are interrupted by three hypervariable regions, HVRs (or CDRs), which are referred to in the art and herein below as “complementarity determining region for “CDR1”; as “complementarity determining region 2” or “CDR2”; and as “complementarity determining region 3” or “CDR3”, respectively. Thus, the general structure or sequence of an immunoglobulin variable domain can be indicated as follows: FR1-CDR1-FR2-CDR2-FR3-CDR3-FR4. The framework regions adopt a beta-sheet conformation and the CDRs may form loops connecting the beta-sheet structure. The CDRs in each chain are held in their three-dimensional structure by the framework regions and form together with the CDRs from the other chain the antigen binding site.

Within the context of this invention, reference to CDR's is based on the definition of CCG, also referred to as IMGT (Lefranc M P, Pommie C, Ruiz M, Giudicelli V, Foulquier E, Truong L, Thouvenin-Contet V, Lefranc G. “IMGT unique numbering for immunoglobulin and T cell receptor variable domains and Ig superfamily V-like domains.” Dev Comp Immunol. 2003 January; 27(1):55-77; Giudicelli V, Brochet X, Lefranc M P. “IMGT/V-QUEST: IMGT standardized analysis of the immunoglobulin (IG) and T cell receptor (TR) nucleotide sequences”. Cold Spring Harb Protoc. 2011; 2011 (6):695-715. An alternative definition of CDRs known in the art is based on Chothia (Chothia and Lesk, J. Mol. Biol. 1987, 196: 901-917), together with Kabat (E. A. Kabat, T. T. Wu, H. Bilofsky, M. Reid-Miller and H. Perry, Sequence of Proteins of Immunological Interest, National Institutes of Health, Bethesda (1983)).

The term “constant domains” or “constant region” as used within the current application denotes the sum of the domains of an antibody other than the variable region. Such constant domains and regions are well known in the state of the art and e.g. described by Kabat et al. (“Sequence of proteins of immunological interest”, US Public Health Services, NIH Bethesda, MD, Publication No. 91).

The “Fe part” or “Fc domain” of an antibody is not involved directly in binding of an antibody to an antigen, but exhibits various effector functions. An “Fe part/domain of an antibody” is a term well known to the skilled artisan and defined on the basis of papain cleavage of antibodies. Depending on the amino acid sequence of the constant region of their heavy chains, antibodies or immunoglobulins are divided in the classes: IgA, IgD, IgE, IgG and IgM. According to the heavy chain constant regions the different classes of immunoglobulins are called a, d, e, g, and m respectively. Several of these may be further divided into subclasses (isotypes), e.g. IgG1, IgG2, IgG3, and IgG4, IgA1, and IgA2. The Fc part of an antibody is directly involved in ADCC (antibody dependent cell-mediated cytotoxicity) and CDC (complement-dependent cytotoxicity) based on complement activation, C1q binding and Fc receptor binding. Complement activation (CDC) is initiated by binding of complement factor C1q to the Fc part of most IgG antibody subclasses.

While the influence of an antibody on the complement system is dependent on certain conditions, binding to C1q is caused by defined binding sites in the Fc part. Such binding sites are e.g. L234, L235, D270, N297, E318, K320, K322, P331 and P329 (numbering according to EU numbering (Edelman et al, Proc Natl Acad Sci USA. 1969 May; 63(1):78-85)). Most crucial among these residues in mediating C1q and Fcgamma receptor binding in IgG1 are L234 and L235 (Hezareh et al, J. Virology 75 (2001) 12161-12168, Shields et al (2001) JBC, 276 (9): 6591-6604). Antibodies of subclass IgG1 and IgG3 usually show complement activation and C1q and C3 binding, whereas IgG2 and IgG4 do not activate the complement system and do not bind C1q and C3.

The term “antibody” or “antibody molecule” (used synonymously herein) encompasses a monoclonal antibody, a polyclonal antibody, a human antibody, a humanized antibody, a chimeric antibody, multispecific antibodies (e.g., bispecific antibodies), a fragment of an antibody, in particular a Fv, Fab, Fab′, or F(ab′)2 fragment. The antibody may have an effector function, such as ADCC or CDC, that is usually mediated by the Fc part (antibody constant region) of the antibody, or it may have no effector function, e.g. by lacking a Fc part or having a blocked, masked Fc part, in essence a Fc part that is not or insufficiently recognized by immune cells or immune system components, like the complement system.

Monoclonal antibodies (mAb) are monospecific antibodies that are identical in amino acid sequence. They may be produced by hybridoma technology from a hybrid cell line (called hybridoma) representing a clone of a fusion of a specific antibody-producing B cell with a myeloma (B cell cancer) cell (Kohler G, Milstein C. Continuous cultures of fused cells secreting antibody of predefined specificity. Nature 1975; 256:495-7.).

Alternatively, monoclonal antibodies may be produced by recombinant expression in host cells (Norderhaug L, Olafsen T, Michaelsen T E, Sandlie I. (May 1997). “Versatile vectors for transient and stable expression of recombinant antibody molecules in mammalian cells.” J Immunol Methods 204 (1): 77-87; see also below).

A “recombinant antibody” or “recombinant binding protein” is an antibody or binding protein which has been produced by a recombinantly engineered host cell. It is optionally isolated or purified.

Full length antibodies can be treated with enzymes such as papain or pepsin to generate useful antibody fragments. Papain digestion is used to produces two identical antigen-binding antibody fragments called “Fab” fragments, each with a single antigen-binding site, and a residual “Fc” fragment. The Fab fragment also contains the constant domain of the light chain and the CH1 domain of the heavy chain. Pepsin treatment yields a F(ab′)2 fragment that has two antigen-binding sites and is still capable of cross-linking antigen. Fab′ fragments differ from Fab fragments by the presence of additional residues including one or more cysteines from the antibody hinge region at the C-terminus of the CH1 domain. F(ab′)2 antibody fragments are pairs of Fab′ fragments linked by cysteine residues in the hinge region. Other chemical couplings of antibody fragments are also known.

“Fv” fragment contains a complete antigen-recognition and binding site consisting of a dimer of one heavy and one light chain variable domain in tight, non-covalent association. In this configuration, the three CDRs of each variable domain interact to define an antigen-biding site on the surface of the VH-VL dimer.

Collectively, the six CDRs confer antigen binding specificity to the antibody.

A “single-chain Fv” or “scFv” antibody fragment is a single chain Fv variant comprising the VH and VL domains of an antibody where the domains are present in a single polypeptide chain. The single chain Fv is capable of recognizing and binding an antigen. The scFv polypeptide may optionally also contain a polypeptide linker positioned between the VH and VL domains in order to facilitate formation of a desired three-dimensional structure for antigen binding by the scFv (see, e.g., Pluckthun, 1994, In The Pharmacology of monoclonal Antibodies, Vol. 113, Rosenburg and Moore eds., Springer-Verlag, New York, pp. 269-315).

A “single-chain Fab” or “scFab” antibody fragment is a single chain Fab variant comprising the VL, CL, VH and CH1 domains of an antibody where the domains are present in a single polypeptide chain. Said polypeptide chain may consist of said domains, consist essentially thereof or comprise them. The single chain Fab is capable of recognizing and binding an antigen. The scFab polypeptide may optionally also contain a polypeptide linker positioned between the CL and VH domains (Hust et al (2007) BMC Biotechnology). To the extent said polypeptide chain comprises said VL, CL, VH and CH1 domains, yet further domains, preferably Ig domains, more preferably CH2 and/or CH3 domains may be present.

For application in man, it is often desirable to reduce immunogenicity of therapeutic molecules, such as antibodies or binding proteins comprising an antigen binding unit as described herein, originally derived from other species, like mouse. This can be done by construction of chimeric antibodies/binding proteins, or by a process called “humanization”. In this context, a “chimeric antibody”; or “chimeric antigen binding unit” is understood to be an antibody or an antigen binding unit comprising a sequence part (e.g. a variable domain) derived from one species (e.g. mouse) fused to a sequence part (e.g. the constant domains) derived from a different species (e.g. human). In this context, a “humanized antibody”, “a humanized binding protein” or a “humanized antigen binding unit” is an antibody, a protein or antigen binding unit comprising a variable domain originally derived from a non-human species, wherein certain amino acids have been mutated to make the overall sequence of that variable domain more closely resemble a sequence of a human variable domain. Methods of humanization of antibodies are well-known in the art (Billetta R, Lobuglio A F. “Chimeric antibodies”. Int Rev Immunol 1993; 10(2-3): 165-76; Riechmann L, Clark M, Waldmann H, Winter G (1988). “Reshaping human antibodies for therapy”. Nature: 332:323).

An “optimized antibody” or an “optimized antigen binding unit or protein” is a specific type of humanized antibody or humanized antigen binding unit/protein which includes an immunoglobulin amino acid sequence variant, or fragment thereof, which is capable of binding to a predetermined antigen and which comprises one or more FRs having substantially the amino acid sequence of a human immunoglobulin and one or more CDRs having substantially the amino acid sequence of a non-human immunoglobulin. This non-human amino acid sequence often referred to as an “import” sequence is typically taken from an “import” antibody domain, particularly a variable domain. In general, an optimized antibody includes at least the CDRs (or HVLs) of a non-human antibody or derived from a non-human antibody, inserted between the FRs of a human heavy or light chain variable domain. It will be understood that certain mouse FR residues may be important to the function of the optimized antibodies and therefore certain of the human germline sequence heavy and light chain variable domains residues are modified to be the same as those of the corresponding mouse sequence. During this process undesired amino acids may also be removed or changed, for example to avoid deamidation, undesirable charges or lipophilicity or non-specific binding. An “optimized antibody”, an “optimized antibody fragment” or “optimized” may sometimes be referred to as “humanized antibody”, “humanized antibody fragment” or “humanized”, or as “sequence-optimized”.

Furthermore, technologies have been developed for creating antibodies or VH/VL domains based on sequences derived from the human genome, for example by phage display or use of transgenic animals (WWW.Ablexis.com/technology-alivamab.php; WO 90/05144; D. Marks, H. R. Hoogenboom, T. P. Bonnert, J. McCafferty, A. D. Griffiths and G. Winter (1991) “By-passing immunisation. Human antibodies from V-gene libraries displayed on phage.” J. Mol. Biol, 222, 581-597; Knappik et al, J. Mol. Biol. 296: 57-86, 2000; S. Carmen and L. Jermutus, “Concepts in antibody phage display”. Briefings in Functional Genomics and Proteomics 2002 1(2): 189-203; Lonberg N, Huszar D. “Human antibodies from transgenic mice”. Int Rev Immunol. 1995; 13(1):65-93.; Bruggemann M, Taussig M J. “Production of human antibody repertoires in transgenic mice”. Curr Opin Biotechnol. 1997 August; 8(4):455-8.). Such antibodies or antigen binding units or VH/VL domains are “human antibodies,” “human antigen binding units,” or “human VH/VL domains” in the context of the present invention.

The term “human antibody”, “human antigen binding unit”, or “human VH/VL domain” as used herein, is intended to include antibodies, antigen binding units or VH/VL domains having variable (and constant, if applicable) regions derived from human germline immunoglobulin sequences. The human antibodies, antigen binding units, proteins or VH/VL domains of the present technology may include amino acid residues not encoded by human germline immunoglobulin sequences (e.g., mutations introduced by random or site-specific mutagenesis in vitro or by somatic mutation in vivo). However, the term “human antibody”, “human antigen binding unit”, or “human VH/VL domain” as used herein, is not intended to include antibodies in which CDR sequences derived from the germline of another (mammalian species), such as a mouse, rat or rabbit, have been grafted onto human framework sequences. Thus, as used herein, the term “human antibody”, “human antigen binding unit”, or “human VH/VL domain” refer to an antibody, antigen binding unit or VH/VL domain in which substantially every part of the protein (e.g., CDR, framework, CL, CH domains (e.g., CH1, CH2, CH3), hinge, VL, VH) is substantially non-immunogenic in humans, with only minor sequence changes or variations. Such changes or variations optionally and preferably retain or reduce the immunogenicity in humans or other species relative to non-modified antibodies or antigen binding units.

Thus, a human antibody, human antigen binding unit or human VH/VL domain is distinct from e.g., a chimeric or humanized antibody. It is pointed out that a human antibody, human antigen binding unit or human VH/VL domain can be produced by a non-human animal or prokaryotic or eukaryotic cell that is capable of expressing functionally rearranged human immunoglobulin (e.g., heavy chain and/or light chain) genes. The term “monomer” refers to a homogenous form of an antibody or a multi-specific protein as described herein. For example, for a full-length antibody, monomer means a monomeric antibody having two identical heavy chains and two identical light chains. In the context of the present invention, a monomer means a protein of the present invention having a single antigen binding unit specific for DLL3, and a single antigen binding unit specific for CD3 as described herein. For example, a monomer of a binding protein described herein may have two chains, a first chain comprising a single chain Fab with a first antigen binding unit and optionally a first Fc domain and a second chain comprising a single chain Fab with a second antigen binding unit and optionally a second Fc domain. An epitope is a region of an antigen that is bound by an antibody or antigen binding moiety (e.g. the antigen binding unit of the proteins described herein). The term “epitope” includes any polypeptide determinant capable of specific binding to an antibody or antigen binding moiety. In certain embodiments, epitope determinants include chemically active surface groupings of molecules such as amino acids, glycan side chains, phosphoryl, or sulfonyl, and, in certain embodiments, may have specific three-dimensional structural characteristics, and/or specific charge characteristics.

Conformational and non-conformational epitopes are distinguished in that the binding to the former but not the latter is lost in the presence of denaturing solvents.

An antigen binding molecule/protein (such as an immunoglobulin, an antibody, an antigen binding unit, or a fragment of such antigen binding molecule/protein) that can“bind”, “bind to”, “specifically bind”, or “specifically bind to”, that “has affinity for”, “is specific for” and/or that “has specificity for” a certain epitope, antigen or protein (or for at least one part, fragment or epitope thereof) is said to be “against” or “directed against “said epitope, antigen or protein or is a “binding” molecule/protein with respect to such epitope, antigen or protein.

As used herein, the terms “binding” and “specific binding” refer to the binding of the antibody or antigen binding moiety (such as an immunoglobulin, an antibody, an antigen binding unit, or a fragment of such antigen binding molecule/protein) to an epitope of the antigen in an in vitro assay, preferably in a plasmon resonance assay ((Malmqvist M., “Surface plasmon resonance for detection and measurement of antibody-antigen affinity and kinetics.”, Curr Opin Immunol. 1993 April; 5(2):282-6.)) with purified wild-type antigen.

Antibody affinity can also be measured using kinetic exclusion assay (KinExA) technology (Darling, R. J., and Brault P-A., “Kinetic exclusion assay technology: Characterization of Molecular Interactions.” ASSAY and Drug Development Technologies. 2004, December 2(6): 647-657).

Generally, the term “specificity” refers to the number of different types of antigens or epitopes to which a particular antigen binding molecule/protein (such as an immunoglobulin, an antibody, an antigen binding unit, or a fragment of such antigen binding molecule/protein) can bind. The specificity of an antigen-binding molecule/protein can be determined based on its affinity and/or avidity. The affinity, represented by the equilibrium constant for the dissociation of an antigen with an antigen-binding protein (KD), is a measure for the binding strength between an epitope and an antigen-binding site on the antigen-binding molecule/protein: the lesser the value of the KD, the stronger the binding strength between an epitope and the antigen-binding molecule/protein (alternatively, the affinity can also be expressed as the affinity constant (KA), which is 1/KD). As will be clear to the skilled person (for example on the basis of the further disclosure herein), affinity can be determined in a manner known per se, depending on the specific antigen of interest. Avidity is the measure of the strength of binding between an antigen-binding molecule/protein (such as an immunoglobulin, an antibody, an antigen binding unit, or fragment of such antigen binding molecule/protein) and the pertinent antigen. Avidity is related to both the affinity between an epitope and its antigen binding site on the antigen-binding molecule/protein and the number of pertinent binding sites present on the antigen-binding molecule/protein.

The term “isolated,” as used herein, refers to material that is removed from its original or native environment (e.g. the natural environment if it is naturally occurring). For example, a naturally-occurring polynucleotide or polypeptide present in a living animal is not isolated, but the same polynucleotide or polypeptide, separated by human intervention from some or all of the co-existing materials in the natural system, is isolated. Such polynucleotides could be part of a vector and/or such polynucleotides or polypeptides could be part of a composition, and still be isolated in that such vector or composition is not part of the environment in which it is found in nature. For example, a nucleic acid, protein/polypeptide molecule is considered to be “(in) essentially isolated (form)”—when compared to its native biological source and/or the reaction medium or cultivation medium from which it has been obtained—when it has been separated from at least one other component with which it is usually associated in said source or medium, such as another nucleic acid, another protein/polypeptide, another biological component or macromolecule or at least one contaminant, impurity or minor component. In particular, a nucleic acid or protein/polypeptide molecule is considered “essentially isolated” when it has been purified at least 2-fold, in particular at least 10-fold, more in particular at least 100-fold, and up to 1000-fold or more. A nucleic acid or protein/polypeptide molecule that is “in essentially isolated form” is preferably essentially homogeneous, as determined using a suitable technique, such as a suitable chromatographical technique, e.g., polyacrylamide-gel electrophoresis.

The term “covalently linked” as used herein means either a direct covalent bond between residues, or an indirect association where two residues are not directly bonded but are both covalently bonded to an intermediate molecule or domain, e.g. an intermediate domain of an immunoglobulin.

It is to be inferred without explicit recitation and unless otherwise intended, that when the present technology relates to a polypeptide, protein, polynucleotide or antibody, an equivalent or a biologically equivalent of such is intended within the scope of the present technology.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in an eukaryotic cell. The expression level of a gene may be determined by measuring the amount of mRNA or protein in a cell or tissue sample.

As used herein the term “biological sample” means a sample material derived from or contacted by living cells. The term is intended to include tissues, cells and biological fluids isolated from a subject. As used herein, the term “tissue sample” shall refer to a cellular sample that preserves the cross-sectional spatial relationship between the cells as they existed within the subject from which the sample was obtained.

Biological samples can also be obtained from biopsies of internal organs or from cancers.

“Histochemistry” and cytochemistry” are techniques used to identify a molecule within the context of intact cells by labeling the samples with an agent that binds specifically to the molecule in a manner than can be visualized on a microscope. “Immunohistochemistry” and “immunocytochemistry” are types of histochemistry and cytochemistry that use antibodies to label the molecules.

“DLL3” has its art-established meaning as defined in the background section herein above which section is part of the disclosure of the present invention.

“CD3” as used herein refers to human CD3 (cluster of differentiation 3). CD3 is a composed of four polypeptide chains, i.e., two CD3 epsilon chains (UniProt: P07766), a CD3 gamma chain (Uniprot: P09693) and a CD3 delta chain (UniProt: P04234). Since T-cells express CD3 on their surface, it is the specificity of proteins of the invention for this antigen which gives rise to the designation of said molecules as “T-cell engagers” or “T-cell engaging protein”. Preferably, TCEs bind to CD3 epsilon.

As used herein, the term “about” may encompass deviations of ±10%, optionally ±5% of the numerical value denoted with this term.

The terms “patient” and “subject” are used interchangeably herein. All subjects, patients or similar denotations refer to a subject, especially a human being, who receives an administration of the T-cell engaging protein and is in need of a treatment with that substance.

As used herein a “a method of treatment of a cancer selected from Neuroendocrine Neoplasms (NEN), glioma, glioblastoma, medullary thyroidal carcinoma, and Merkel cell carcinoma” means that a medical indication for administering the T-cell engaging molecule is present in these indications.

As used herein, the term “week” is defined as a time period encompassing 7 days. However, if the last day of the week is a holiday, the end of the 7 days period may be the day before or after the holiday or even after said holiday. Therefore, the term “week” in general encompasses 7 days but may be altered within the criteria mentioned.

The term “cycle” as used herein refers to one or more than one such as three administrations. A cycle may optionally be repeated. Conceptually, this disclosure distinguishes between a “step-in cycle” and a “treatment cycle”. The step-in cycle, as e.g., subject of the first aspect of this invention, involves at least three administrations. Apart from exceptional cases discussed herein, said step-in cycle is not repeated. A treatment cycle on the other hand may be, and preferably is, repeated. Repetitions are preferably performed to secure tumor control or eradication. Preferred numbers of repetitions of treatments cycles are disclosed herein. As regards the number of doses/administrations to be given within a single treatment cycle, preference is given to three for first treatment cycles, and to one for second treatment cycles.

An administration on a certain day of a step-in or a treatment cycle, such as “day 1”, of a three weeks' time period may also admit a tolerance of two, or optionally one day, before or after that day, because a patient may not be able to appear at the exact appointment for various reasons. In addition, depending on the nomenclature, “day 1” of a step-in or treatment cycle can also be denoted as “day 0” and all following days are adapted accordingly.

The term “directly follows” as used to define the succession of cycles (first treatment cycle following step-in cycle, second treatment cycle following first treatment cycle) means that there is no interruption between cycles. To explain further, where a last dose within a given cycle is to be administered in a given week, the first dose of the respective subsequent cycle is to be given in the week directly following said given week. A preferred time to elapse is a week or seven days. To give an example: the 1st administration (cycle1 day1) occurs on a Tuesday; the 2nd administration (cycle1 day8) will occur on Tuesday the following week; the 3rd administration (cycle1 day15) will occur on Tuesday one week after; and the next administration (cycle2 day1) will occur on Tuesday one week (7 days) after the 3rd administration in cycle1. In this example, “cycle1” may refer to the step-in cycle, and “cycle2” may refer to the first treatment cycle; or “cycle1” may refer to the first treatment cycle, and “cycle2” may refer to the second treatment cycle.

As used herein, a “regimen” is a totality of all cycles, i.e., step-in cycle and first and following treatment cycles, when treating a patient.

As used herein an “administration” of the T-cell engaging protein is usually carried out within one treatment day and not over several days. A preferred administration route is intravenous (i.v.). A preferred duration of an i.v. administration is between about 0.5 and about 5 hours, more preferably between about 1 and about 3 hours such as about 2 hours.

A cytokine release syndrome (CRS) may be classified by a Common Toxicity Criteria (NCI CTC) grade throughout this disclosure. A preferred grading scheme for both CRS and ICANS is the ASTCT consensus grading as described in Lee et al., Biol Blood Marrow Transplant 25, 625-638 (2019). Response is evaluated according to Response Evaluation Criteria In Solid Tumours (RECIST, version 1.1; Eisenhauer E A et al., New response evaluation criteria in solid tumours: revised RECIST guideline (version 1.1). Eur J Cancer 45, 228-247 (2009).

### Contributions Made by the Present Invention

The following is a non-exhaustive description of the contribution to the art made by the present inventors. T-cell engagers, upon administration to a patient in need thereof, may trigger severe or potentially fatal adverse responses in a patient. These responses include a syndrome commonly referred to as cytokine release syndrome (CRS) or cytokine storm. Without wishing to be bound by a specific theory, it is considered that the CD3 specificity of said engagers is a feature contributing to said adverse responses.

The present inventors addressed this problem.

The dosage regimens of the invention surprisingly allow to reduce the frequency and severity of adverse events, such as cytokine release syndrome (CRS) and ICANS and/or reach therapeutic doses (target dose) as fast as possible.

In more detail, while not wishing to be bound by a specific theory:

Because the CRS risk (frequency, severity) at initial administrations is correlated with the dose of the T cell engaging (TCE) protein, and furthermore because it is considered that said risk is correlated with body weight, the doses in the course of the step-in cycle are adapted to the body weight of said patient. Therefore, at least one dose of the step-in cycle, typically the initial dose, is adapted to the body weight of said patient.

Because furthermore the CRS risk at a certain dose decreases with repeated administration, the body weight adapted dose of the T-cell engaging protein is increased stepwise.

This is reflected in the first aspect of the present invention.

In addition to TCE dose and administration number (initial dose or administration, elevated/further dose or administration in the course of the step-in cycle) individual patient factors might be relevant for the CRS risk, including e.g., target expression (DLL3 expression in this specific case), immune environment (immune cells in the tumour) and tumour load (optionally or where applicable, metastasis load). The present inventors gave preference to determining and addressing said patient specific CRS risk based on clinical symptoms as observed upon administration of a dose (initial, elevated and/or further dose) within said step-in cycle.

Therefore, such dose to be administered within said step-in cycle may be repeated, to the extent necessary, in order to secure sufficient cytokine depletion such that a subsequent dose may be further escalated. Such determination of said clinical symptoms requires preferably the careful attention of a clinician, preferably monitoring any onset of said CRS, preferably in accordance with the scoring schemes as defined herein and/or established in the art.

This is reflected in preferred embodiments of the invention.

In an alternative embodiment said CRS risk of individual patients may be determined by means established in the art such as scanning methods (to determine tumour load and, where applicable, said metastasis load) including ultrasonic and are radiographic methods, the latter preferably upon administration of suitable contrast agents, laboratory assessments or algorithms integrating vital signs (e.g., body temperature, heart rate, breathing rate etc).

The above-discussed design of the step-in cycle has surprisingly been found out to open an avenue for unprecedently high doses in the course of any treatment cycle to follow the step-in cycle while controlling or avoiding adverse effects such as CRS.

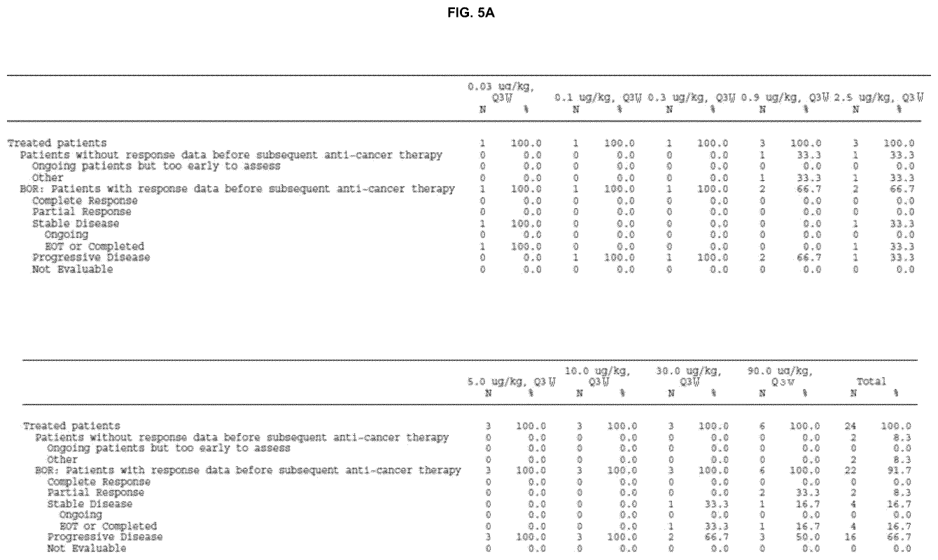

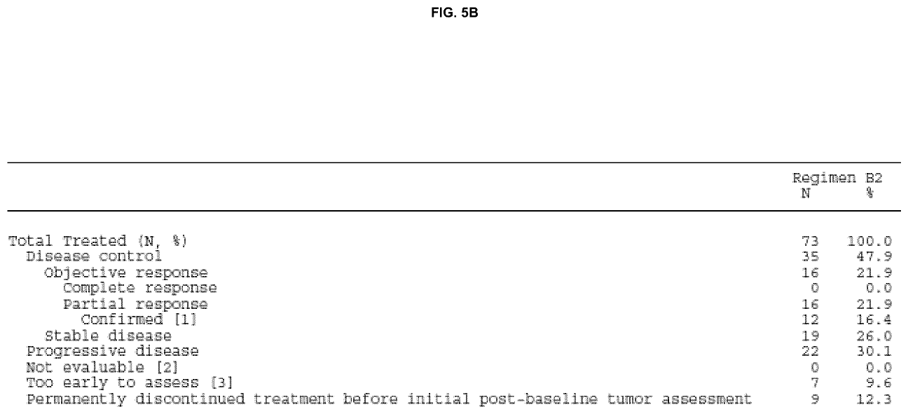

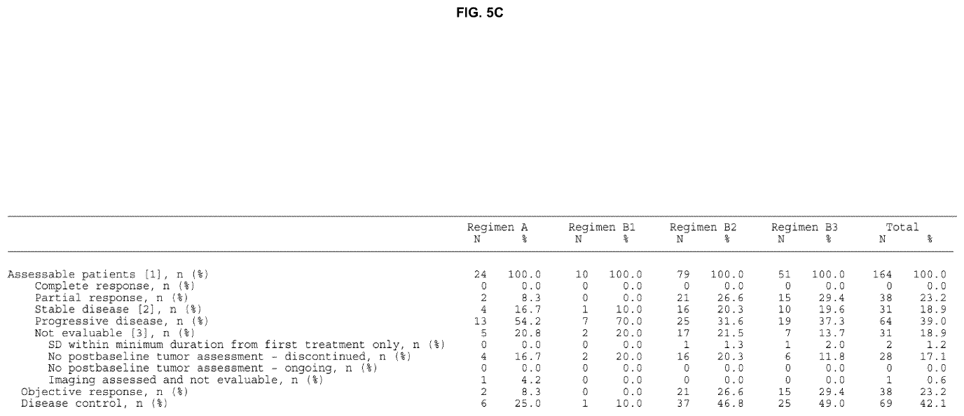

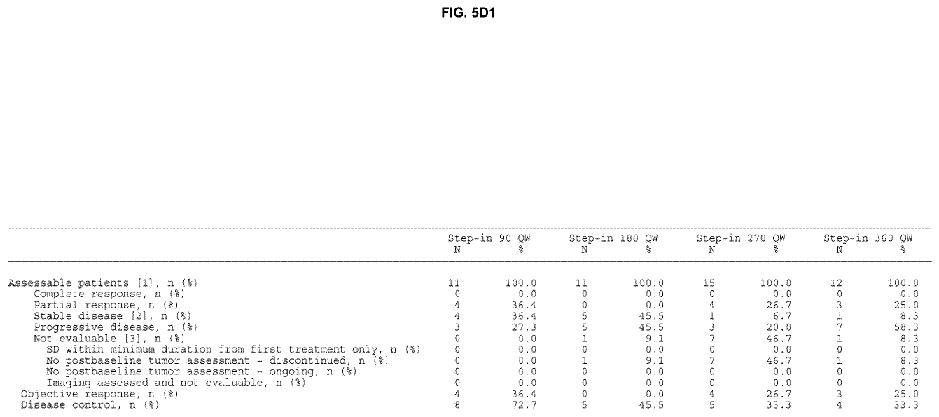

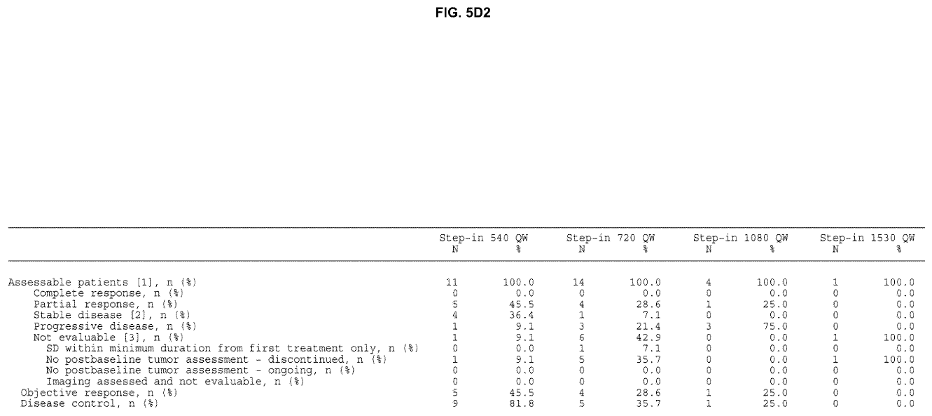

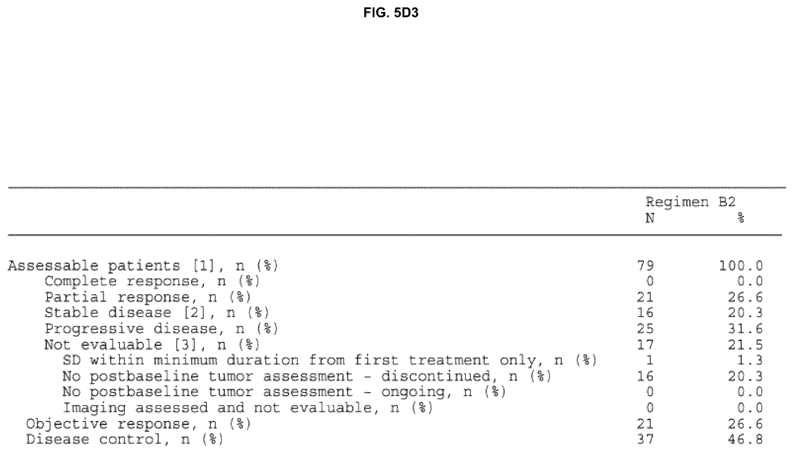

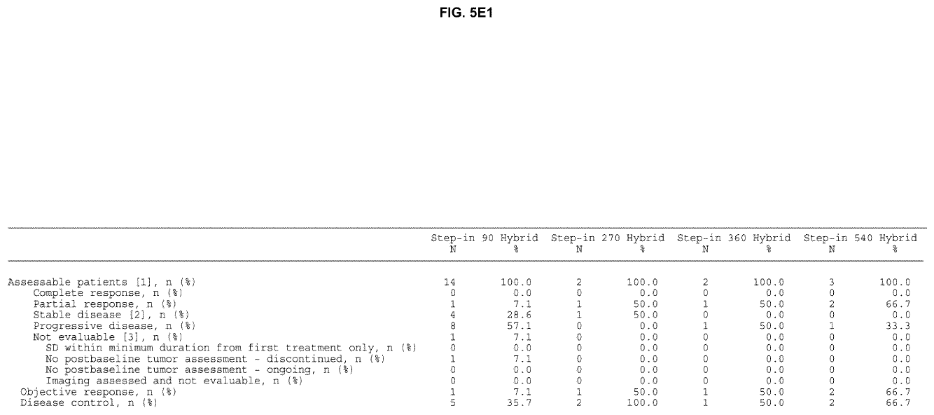

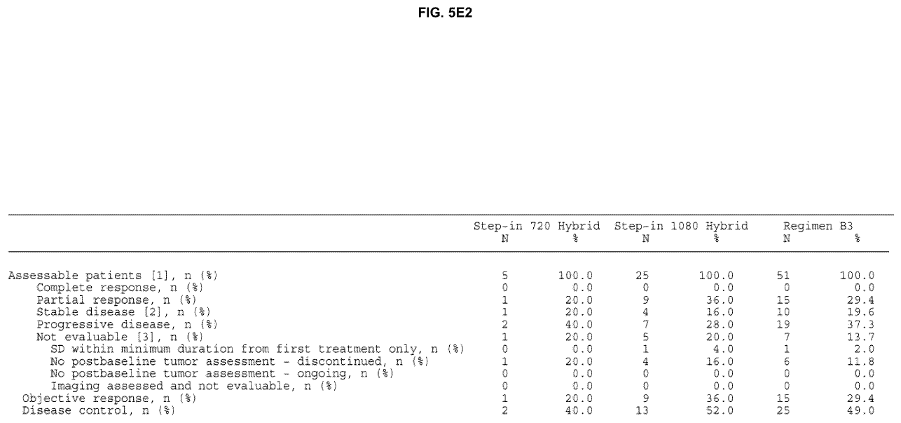

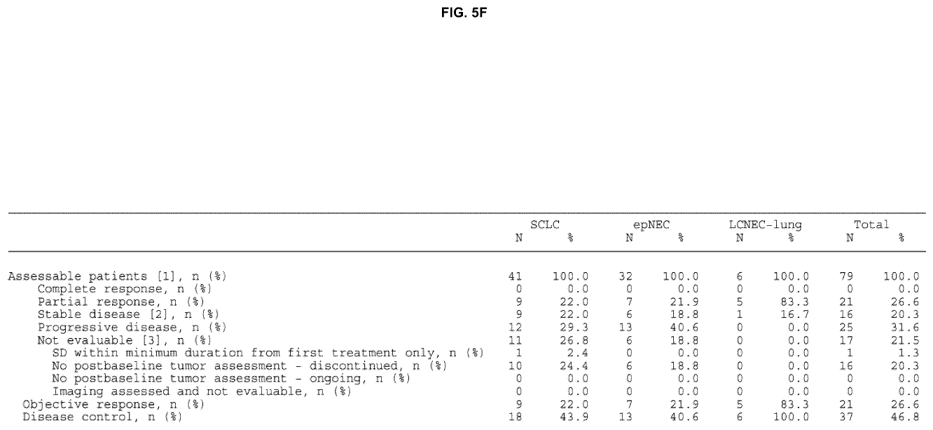

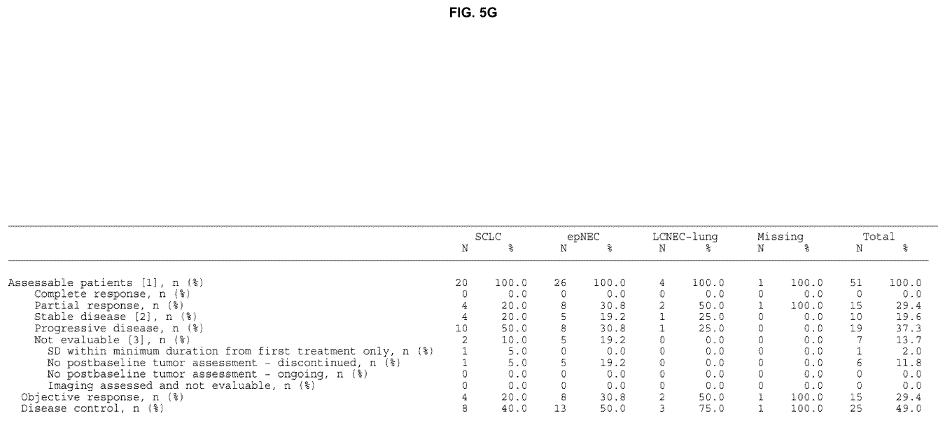

Based on observations in the Phase I trial BI 764532 described in the examples exhibits a wide therapeutic window for weight-based target doses, thus allowing for introduction of a flat dose (non weight-based) regimen for target doses. Clinical responses were observed starting at 90 μg/kg and then also at higher dose levels at similar rates (FIG. 5H). Thus, a flat dose-response relationship is assumed at tested doses. In addition, safety of BI 764532 seems largely determined by initial (step-in) doses, rather than the target dose. Specifically, data from the Phase I trial indicate that nearly all CRS events occur at step-in doses. As no significant difference in incidence or severity of CRS or other Adverse Events (AEs) was observed for the tested target doses, the dose-toxicity relationship appears flat in this range.

A flat dose of 10 mg will be used for the BI 764532 “low dose” regimen. Clinical activity of BI 764532 was first observed at the 90 μg/kg dose level in the 1438-0001 trial. The 10 mg flat dose is proposed as this dose would allow drug exposures to exceed the corresponding exposures at the weight-based dose of 90 μg/kg for 95% percentiles of the patient population in the Phase I trial.

For the BI 764532 “high dose” regimen, a flat dose of 60 mg will be used. Data from the Phase I trial supports the assumption that this dose would lead to drug exposure similar to the corresponding exposures for a weight-based dose of 720-1080 μg/kg for 95% percentiles of the patient population in the Phase I trial.

In terms of further evidence, with the dosing regimens according to the methods of the present invention, both efficacy and/or tumor shrinkage were observed while avoiding or controlling CRS.

### EMBODIMENTS

Of note, this disclosure makes use of short-hand identifiers such as B2, B3, B4a-B4d and Dareon™-5 for preferred dosage regimens. The actual doses to be administered and intervals to be observed are defined in the general description and the claims and exemplified in the Examples.

Said preferred dosage regimens are encompassed by the first aspect of the present invention relating to a T-cell engaging protein comprising a first antigen binding unit that specifically binds to DLL3 and a second antigen binding unit that specifically binds to CD3 for use in a method of treatment of a DLL3-positive cancer or a cancer selected from a Neuroendocrine Neoplasm (NEN), Merkel cell carcinoma (MCC), medullary thyroidal carcinoma and glioma,
the method comprising a step-in cycle, said step-in cycle comprising the steps of:
   administering to a subject an initial dose of said T-cell engaging protein, said initial dose ranging from about 5 μg/kg of the subject's body weight to about 40 μg/kg of the subject's body weight,
   administering to the subject an elevated dose, which is elevated as compared to said initial dose, of said T-cell engaging protein, the elevated dose ranging from about 15 μg/kg of the subject's body weight to about 100 μg/kg of the subject's body weight, or the elevated dose ranging from about 5 mg to about 20 mg, and
   administering to the subject a further dose of the T-cell engaging protein, said further dose ranging from about 80 μg/kg of the subject's body weight to about 1600 μg/kg of the subject's bodyweight, wherein said further dose is equal to or higher than said elevated dose or said further dose is ranging from about 5 mg to about 70 mg.

For the avoidance of doubt, the disclosure of the first aspect is combinable which each of the following preferred embodiments of the following sub-sections of the detailed description.

### Dosing Regimen B2

In a third aspect, the present invention provides a T-cell engaging protein comprising a first antigen binding unit specifically binding to DLL3 and a second antigen binding unit specifically binding to CD3 for use in a method of treatment of a DLL3-positive cancer or a cancer selected from a Neuroendocrine Neoplasm (NEN), Merkel cell carcinoma (MCC), medullary thyroidal carcinoma and glioma,
the method comprising a step-in cycle, said step-in cycle comprising the steps of:
   administering to a subject an initial dose of said T-cell engaging protein, said initial dose ranging from about 20 μg/kg of the subject's body weight to about 40 μg/kg of the subject's body weight,
   administering to the subject an elevated dose, which is elevated as compared to said initial dose, of said T-cell engaging protein, the elevated dose ranging from about 60 μg/kg of the subject's body weight to about 100 μg/kg of the subject's body weight,
   administering to the subject a further dose of the T-cell engaging protein, said further dose ranging from about 80 μg/kg of the subject's body weight to about 1600 μg/kg of the subject's bodyweight, wherein said further dose is equal to or higher than said elevated dose.

Of note, this and further aspects of the invention, in one alternative refers to a cancer which is DLL3 positive. While recognizing that “DLL3 positive” is a characterization in molecular terms, it should not go unnoticed that with the advent of molecular medicine, the characterization of a disorder in molecular terms (markers, causally involved molecules as well as molecules qualifying as a target for therapeutic intervention) has become common. Referring to the term “DLL3 positive” it is of note that there is a significant body of evidence that certain tumors, in particular those which are defined in clinical terms herein, do express DLL3 and are thus considered DLL3-positive. As such, terms “DLL3 positive” and “known to express DLL3” are considered to be equivalent.

As regards the notion of “DLL3 positive” on the one hand and the clinical indications (such as NEN, glioma etc.) on the other hand being presented alternatives, it is understood that in scientific terms these are not considered to be alternatives, let alone mutually exclusive. To the contrary, and as explained herein, said clinical indications are considered to be inherently DLL3 positive. On the other hand, it is conceivable that further clinical indications (beyond these recited herein) are or will be found to be DLL3 positive. Any such further indications would be amenable to treatment in accordance with the invention.

For the avoidance of doubt, it is understood that said subject is a subject in need of said treatment (also referred to as “patient” herein).

Once the step-in cycle in accordance with the present invention has been completed in a given patient using a given T-cell engaging protein, treatment with any T-cell engaging protein may ensue. Preference is given, though, to perform the treatment cycle(s) with the same T-cell engaging protein that has been used for the step-in cycle.

Optional combination treatments are disclosed herein. In wider sense, the term “combination therapy” also embraces the concomitant administrations of two or more TCEs.

Of note, said further dose to be administered within said step-in cycle may already be the dose to be administered within the preferably ensuing treatment cycle. There is no requirement in that respect, though, in case the clinical symptoms of the patient permit such administration, the final dose to be administered within said step-in cycle may be viewed as a first treatment dose—although formally subsumed, in adherence to the nomenclature used herein, under the term “step-in cycle”.

A preferred initial dose is between about 25 and about 35 μg/kg such as about 30 μg/kg.

A preferred elevated dose is between about 80 and 100 μg/kg such as about 90 μg/kg. In that respect it is of note that beneficial effects in terms of treatment may already occur in response to said elevated dose, noting that about 90 μg/kg are considered as the minimal effective dose in terms of treatment.

In other preferred embodiments, said further dose is about 90 μg/kg, about 180 μg/kg, about 270 μg/kg about 360 μg/kg, about 540 μg/kg, about 720 μg/kg, about 1080 μg/kg or about 1530 μg/kg of the subject's body weight. To the extent these values are lower than preferred target doses to be administered in the first treatment cycle as specified below, these lower values may be applied dependent on the clinical symptoms of the patient and/or serve to avoid the onset of severe side effects such as CRS and ICANS, severity preferably being defined in terms of the respective scoring schemes as defined further below.

In a more preferred embodiment, the initial dose is about 30 μg/kg, the elevated dose is about 90 μg/kg and the further dose is about 90 μg/kg of the subject's body weight. In an alternative more preferred embodiment, the initial dose is about 30 μg/kg, the elevated dose is about 90 μg/kg and the further dose is about 180 μg/kg of the subject's body weight. In an alternative more preferred embodiment, the initial dose is about 30 μg/kg, the elevated dose is about 90 μg/kg and the further dose is about 270 μg/kg of the subject's body weight. In an alternative more preferred embodiment, the initial dose is about 30 μg/kg, the elevated dose is about 90 μg/kg and the further dose is about 360 μg/kg of the subject's body weight. In an alternative more preferred embodiment, the initial dose is about 30 μg/kg, the elevated dose is about 90 μg/kg and the further dose is about 540 μg/kg of the subject's body weight. In an alternative more preferred embodiment, the initial dose is about 30 μg/kg, the elevated dose is about 90 μg/kg and the further dose is about 720 μg/kg of the subject's body weight. In an alternative more preferred embodiment, the initial dose is about 30 μg/kg, the elevated dose is about 90 μg/kg and the further dose is about 1080 μg/kg of the subject's body weight. In an alternative more preferred embodiment, the initial dose is about 30 μg/kg, the elevated dose is about 90 μg/kg and the further dose is about 1530 μg/kg of the subject's body weight.

It is understood that any of these specific values, in addition to being preferred specific values, also define upper and/or lower boundaries of preferred sub-intervals. This applies throughout this disclosure.

As such said further dose is preferably between about 90 and about 1530 μg/kg including between about 180 μg/kg and about 1080 μg/kg or between about 360 and about 720 μg/kg.

In those cases where treatment tolerability is good, said initial dose is given once, said elevated dose is also given once, and said further dose, also to be given once, is a preferred target dose as defined in the context of the first treatment cycle.

On the other hand, and as noted above, it is a contribution made by the present invention to accommodate more complicated situations where initial and/or elevated dose are not well tolerated. For more details in that respect, reference is made to the section entitled “Contributions made by the present invention” herein above.

Accordingly, in further embodiments, said initial dose, said elevated dose and/or said further dose is/are administered once; or said initial dose, said elevated dose and/or said further dose is/are administered more than once, optionally 2 to 10 times,

wherein optionally the number of administrations is determined based on presence and/or degree of a cytokine release syndrome (CRS) and/or of an immune effector cell-associated neurotoxicity syndrome (ICANS),
and wherein further optionally said elevated dose follows the initial dose only if said CRS, to the extent determined, is rated grade 1 or less in terms of NCI CTC grade and/or if said ICANS, to the extend determined, is rated grade 1 or less, and/or wherein further optionally said further dose follows the elevated dose only if said CRS, to the extent determined, is rated grade 1 or less in terms of NCI CTC grade and/or if said ICANS, to the extend determined, is rated grade 1 or less. In other words, said initial dose may be administered more than once, optionally 2 to 10 times, in view of the above NCI CTC or ICANS criteria.

This means that the initial dose may be repeated before an elevated dose can be administered. Preferably, such repetition is done once in a week. The administrations and repetitions may be spaced apart a week.

More preferably, said initial dose may be repeated 2, 3, 4, 5, 6, 7, 8, 9 or 10 times. In addition or alternatively, the same also applies for the elevated dose before the further dose is administered within the step-in cycle. Yet further, also said further dose may be repeated, e.g., 2, 3, 4, 5, 6, 7, 8, 9 or 10 times where deemed necessary in view of adverse responses.

Moreover, in case adverse responses occur, alternatively or in addition to repeating a dose (initial dose or elevated dose), a given dose may be reduced as compared to the preceding initial or elevated dose, e.g., to about 10% to about 90% of the preceding dose such about ⅔ or about ⅓, preferably about 50% of the preceding dose. Of note, the option of dose reduction does not only apply to the step-in cycle, but also to any of the treatment cycles disclosed herein.

In preferred embodiments, said initial dose is administered three times to ten times within the step-in cycle, preferably 3, 4, 5, 6, 7, 8, 9 or 10 times. Alternatively or in addition, the same also applies to the elevated dose.

In preferred embodiments, the administration of the doses may be spaced one week apart.

In preferred embodiments, said initial dose is administered three times within three weeks, optionally on day 1, day 8 and day 15 of the step-in cycle, if it is administered for three times. For example, the elevated dose may then be administered on day 21 of the step-in cycle, if the above NCI CTC and/or ICANS criteria are fulfilled. If the initial dose is administered 2 times, it may be administered on day 1 and day 8 in the step-in cycle, wherein the elevated dose follows in day 15. The same considerations apply to the elevated dose. If, after administration of the elevated dose, the NCI CTC and/or ICANS criteria are fulfilled, the further dose is administered next.

In some embodiments, said step-in cycle has a duration of at least three weeks. In preferred embodiments, said step-in cycle may have a duration of three weeks, wherein optionally the initial dose may be administered on day 1, the elevated dose may be administered on day 8 and the further dose may be administered on day 15. Depending on how many times the initial and/or the first dose may be administered, the step-in cycle may have a longer duration than three weeks.

In some embodiments, said initial, elevated and further doses are administered within three weeks, said initial dose being administered once, wherein optionally said initial dose is administered on day 1, said elevated dose is administered on day 8 and said further dose is administered on day 15 of said step-in cycle.

To the extent reference is made to specific days such as day 1, day 8, day 15 etc. it is to be understood that these are particularly preferred days which, however, allow for certain deviations depending on the patient's compliance or availability as well as the attending clinician's capacities. In other words, administrations on, say, days 1, 9 and 14 are within the scope of the present invention. Generally speaking, it will be sufficient to administer once per week regardless of the specific day within a given week. More preferably, deviations from the most preferred scheme (days 1, 8, 15 etc.) should not exceed three, two or one days in either direction.

The above considerations relating to the intervals between subsequent administrations apply generally, i.e., not only to the step-in cycle but also to treatment cycles as disclosed further below.

In some embodiments, said initial dose ranges from about 25 μg/kg of the subject's body weight to about 35 μg/kg of the subject's body weight, and optionally said initial dose is about 30 μg/kg of the subject's body weight;
said elevated dose ranges from about 85 μg/kg of the subject's body weight to about 95 μg/kg of the subject's body weight, and optionally said elevated dose is about 90 μg/kg of the subject's body weight; and said further dose ranges from about 85 μg/kg of the subject's body weight to about 1550 μg/kg of the subject's body weight, and optionally the further dose is about 90 μg/kg, about 180 μg/kg, about 270 μg/kg, about 360 μg/kg, about 540 μg/kg, about 720 μg/kg, about 1080 μg/kg or about 1530 μg/kg of the subject's body weight.

In some embodiments, the method further comprises a first treatment cycle following the step-in cycle, said first treatment cycle comprising the steps of:
administering to a subject a first target dose of said T-cell engaging protein, said first target dose ranging from about 80 μg/kg of the subject's body weight to about 1600 μg/kg of the subject's body weight,
administering to the subject a second target dose of said T-cell engaging protein, said second dose ranging from about 80 μg/kg of the subject's body weight to about 1600 μg/kg of the subject's body weight, and
administering to the subject a third target dose of said T-cell engaging protein, said third dose ranging from about 80 μg/kg of the subject's body weight to about 1600 μg/kg of the subject's bodyweight, wherein optionally the amount of all three target doses is identical. Preferably, at least one of the first, second and third target doses is about 90 μg/kg, about 180 μg/kg, about 270 μg/kg, about 360 μg/kg, about 540 μg/kg, about 720 μg/kg, about 1080 μg/kg or about 1530 μg/kg of the subject's body weight. More preferably, said first, second and third target doses are identical about 90 μg/kg, about 180 μg/kg, about 360 μg/kg, about 540 μg/kg, about 720 μg/kg, about 1080 μg/kg or about 1530 μg/kg of the subject's body weight.

As stated further above, any specific values, be they expressed as bodyweight dependent or flat doses, define also lower or upper limits of preferred sub-intervals, such sub-intervals including for said target dose from about 90 to about 1530 μg/kg or from about 180 to about 1080 μg/kg. In general, there will be a preference for high target doses, noting that the specific design of the step-in cycle will usually allow for the administration of such high target doses (such as about 1080 or about 1530 μg/kg). Yet, and in cases where despite a tailored step-in cycle treatment tolerability is still limited during the treatment cycle(s), a reduction of the target dose to intermediate values such between about 90 and about 800 or between about 90 and about 720 μg/kg may be performed.

Of note, the dependence of a given dose on the patient's body weight during the step-in cycle confers distinct advantages. In particular, such dosing provides improved tolerability including reduced frequency and/or severity of CRS, and furthermore allows for higher target doses in the course of treatment cycles. Without wishing to be bound by a specific theory, it is considered that these advantageous effects results from a controlled cytokine depletion during the step-in cycle.

In accordance with the above, in some embodiments, the method further comprises a first treatment cycle following said step-in cycle, said first treatment cycle comprising the steps of:
administering to a subject a first target dose of the T-cell engaging protein, said first target dose ranging from about 5 mg to about 70 mg,
administering to the subject a second dose of the T-cell engaging protein, said second dose ranging from about 5 mg to about 70 mg, and
administering to the subject a third dose of the T-cell engaging protein, said third dose ranging from about 5 mg to about 70 mg,
   wherein optionally the amount of all three target doses is identical.

In some embodiments, at least one of said first, second or third target doses of said first treatment cycle comprises more than 30 mg.

In some embodiments, at least one of said first, second or third target doses of the first treatment cycle is about 5 mg, about 10 mg, about 20 mg, about 30 mg, about 40 mg, about 50 mg, about 60 mg, or about 70 mg, optionally about 10 mg or about 60 mg.

In preferred embodiments, the amount of all three target doses are identical and about 10 mg. In case of safety concerns, said amount may be further reduced and be in the range of about 5 mg to about 10 mg, and is in particular about 5 mg.

In alternative preferred embodiments, the amount of all three target doses are identical and about 60 mg. In case of safety concerns, said amount may be further reduced and be in the range of about 20 mg to about 60 mg, and is in particular about 30 mg.

In preferred embodiments, said first treatment cycle is not repeated or said first treatment cycle is repeated at least once. Preferred numbers of repetitions are disclosed herein and exemplified in the Examples.

In preferred embodiments, said first, second and third target doses are administered within three weeks of said first treatment cycle, wherein optionally said first target dose is administered on day 1, the second target dose is administered on day 8 and the third target dose is administered on day 15 of said first treatment cycle, wherein optionally said first treatment cycle has a duration of three weeks. Reference is made to a certain flexibility regarding intervals or specific treatment days as explained further above in the context of the description of the step-in cycle.

In preferred embodiments, said first treatment cycle is followed by a second treatment cycle, said second treatment cycle comprising the step of or consisting of the step:
administering to the subject a target dose of said T-cell engaging protein, said target dose ranging from about 80 μg/kg of the subject's body weight to about 1600 μg/kg of the subject's body weight;
   wherein said second treatment cycle is optionally repeated at least once. Preferably, the target dose is about 90 μg/kg, about 180 μg/kg, about 360 μg/kg, about 540 μg/kg, about 720 μg/kg, about 1080 μg/kg or about 1530 μg/kg of the subject's body weight. More preferably, said target dose is about 90 μg/kg, about 180 μg/kg, about 360 μg/kg, about 540 μg/kg, about 720 μg/kg, about 1080 μg/kg or about 1530 μg/kg of the subject's body weight.

In preferred embodiments, said the first treatment cycle is followed by a second treatment cycle, said second treatment cycle comprising the step of or consisting of the step:
administering to the subject a target dose of the T-cell engaging protein, said target dose ranging from about 5 mg to about 70 mg;
   wherein said second treatment cycle is optionally repeated at least once. Preferably, said target dose is about 10 mg or about 60 mg.

In preferred embodiments, said second treatment cycle has a total duration of three weeks and/or said target dose is administered on day 1 of said three weeks. Reference is made to optional or preferred repetitions of treatments cycle as disclosed herein.

In other words, if a patient is considered to be in need of further treatment after the first treatment cycle, the burden on the patient can be reduced by not requiring any more that the patient presents itself once a week in the clinic, but in larger intervals, a preferred larger interval including, but not being confined to three weeks as is subject of the preferred embodiment above. Alternatively, also intervals of about two weeks or every ten or every 20 days may be considered.

In preferred embodiments, said first treatment cycle directly follows said step-in cycle; and/or said second treatment cycle directly follows said first treatment cycle. To explain further, a dosage regimen comprising step-in cycle as well as first and second treatment cycle may involve administrations on days 1, 8, 15, 22, 29, 36, 43, 64, 85 etc., wherein days 1, 8 and 15 would correspond to the step-in cycle (step-in doses being tolerable in this example such that no repetitions are required), days 22, 29, 36 (counting continued from the day of administration of the first dose of the step-in cycle) would correspond to one application of the first treatment cycle (weekly target dose), and days 43, 64, 85 etc. would correspond to repeated (here three) applications of the second treatment cycle (3 week administration intervals in this example).

In some embodiments, essentially no cytokine release syndrome occurs after said elevated dose of said step-in cycle in the subject.

This would generally be scenario where the patient under consideration would qualify for said further dose of said step-in cycle being the same as a preferred target dose of said first treatment cycle. Optionally, premedication may be adjusted under such circumstances as disclosed herein.

In some embodiments, said T-cell engaging protein is the only anti-cancer agent to be administered.

In some embodiments, at least one further anti-cancer agent is to be administered, said further anti-cancer agent preferably being a chemotherapeutic agent. Preferred chemotherapeutic agents are those disclosed in the background section herein above which is part of the disclosure of the invention as well as those referred to in the Examples.

In certain embodiments, the subject has been treated with a chemotherapy involving administration of inhibitors of cell growth and/or division prior to the step-in cycle.

In certain embodiments, an occurrence of cytokine release syndrome is to be treated with an anti-cytokine release syndrome agent, preferably selected from the group consisting of a corticosteroid, an IL-6 antagonist and an IL-6R antagonist.

Overall, preference is given to treatment cycles of 3 weeks duration.

In some embodiments, preferred is a regimen within a method of treatment as illustrated in Table 1A below.

TABLE 1A
Embodiment of an aspect of the present invention.
Regimen B2:
One cycle duration = 3 weeks
Step-in cycle as defined below
First treatment cycles: Target doses administered weekly
from d 1 of the first treatment cycle until progression
or toxicity, preferably a maximum of 36 months.
Of note, there is no second treatment cycle in this
particular regimen, but treatment cycle 1 is repeated.

| | Step-in cycle | | | First treatment cycles (repeated) | | |
|---|---|---|---|---|---|---|
| | d 1 | d 8 | d 15 | d 1 | d 8 | d 15 |
| dose | 30 μg/kg | 90 μg/kg | Target dose: | Target dose: | Target dose: | Target dose: |
| | | | from 90 to | from 90 to | from 90 to | from 90 to |
| | | | 1530 μg/kg | 1530 μg/kg | 1530 μg/kg | 1530 μg/kg |

### Dosing Regimen B3

In a fourth aspect, the present invention provides T-cell engaging protein comprising a first antigen binding unit specifically binding to DLL3 and a second antigen binding unit specifically binding to CD3 for use in a method of treatment of a DLL3-positive cancer or a cancer selected from a Neuroendocrine Neoplasm (NEN), Merkel cell carcinoma (MCC), medullary thyroidal carcinoma and glioma,
the method comprising a step-in cycle, the step-in cycle comprising the steps of:
   administering to the subject an initial dose, an elevated dose, which is elevated as compared to said initial dose, and a further dose of said T-cell engaging protein, wherein a ratio between said initial, elevated and further dose per kilogram of the subject's body weight is about 1: about 3: about 9.

In a preferred embodiment, said initial dose is about 10 μg/kg, said elevated dose is about 30 μg/kg, and said further dose is about 90 μg/kg.

Otherwise, the preferred embodiments of the third aspect of the invention, set forth under sub-section “Dosing Regimen B2” apply mutatis mutandis.

In particular, a duration of three weeks is preferred, in particular in those cases where there is good treatment tolerability. In that context, administration at days 1, 8 and 15 is preferred.

In a fifth aspect, the present invention provides a T-cell engaging protein comprising a first antigen binding unit specifically binding to DLL3 and a second antigen binding unit specifically binding to CD3 for use in a method of treatment of a DLL3-positive cancer or a cancer selected from a Neuroendocrine Neoplasm (NEN), Merkel cell carcinoma (MCC), medullary thyroidal carcinoma and glioma,
the method comprising a step-in cycle, the step-in cycle comprising the steps of:
   administering to a subject an initial dose of said T-cell engaging protein, said initial dose ranging from about 5 μg/kg of the subject's body weight to about 15 μg/kg of the subject's body weight,
   administering to the subject an elevated dose, which is elevated as compared to said initial dose, of said T-cell engaging protein, the elevated dose ranging from about 20 μg/kg of the subject's body weight to about 40 μg/kg of the subject's body weight,
   administering to the subject a further dose of said T-cell engaging protein, said further dose ranging from about 80 μg/kg of the subject's body weight to about 1600 μg/kg of the subject's bodyweight.

In the following, preferred embodiments of the above second and third aspects of the invention are given. Of note, additional preferred embodiments as well as explanation as given further above in the context of the first aspect of the invention and preferred embodiments thereof apply mutatis mutandis also to the second, third and any further aspects of the invention.

In some embodiments, said further dose is equal to a target dose, said target dose being the dose to be administered during a first treatment cycle which optionally directly follows said step-in cycle. In preferred embodiments, the further dose and the target dose are about 90 μg/kg, about 180 μg/kg, about 270 μg/kg, about 360 μg/kg, about 540 μg/kg, about 720 μg/kg, about 1080 μg/kg or about 1530 μg/kg of the subject's body weight. More preferably, the further dose and the target dose are about 1080 μg/kg or about 1530 μg/kg of the subject's body weight.

In some embodiments, said initial dose, said elevated dose and/or said further dose is/are administered once; or
said initial dose, said elevated dose and/or said further dose is/are administered more than once, optionally 2 to 10 times,
wherein optionally the number of administrations is determined based on presence and/or degree of a cytokine release syndrome (CRS) and/or of an immune effector cell-associated neurotoxicity syndrome (ICANS),
and wherein further optionally said elevated dose follows the initial dose only if said CRS, to the extent determined, is rated grade 1 or less in terms of NCI CTC grade and/or if said ICANS, to the extend determined, is rated grade 1 or less, and/or wherein
further optionally said further dose follows the elevated dose only if said CRS, to the extent determined, is rated grade 1 or less in terms of NCI CTC grade and/or if said ICANS, to the extend determined, is rated grade 1 or less.

In some embodiments, said initial dose, said elevated dose and/or said further dose is/are administered three times to ten times within the step-in cycle.

In some embodiments, said initial dose is administered three times within three weeks, optionally on day 1, day 8 and day 15 of the three weeks.

In some embodiments, said step-in cycle has a duration of at least three weeks.

In some embodiments, said initial, elevated and further doses are administered within three weeks, said initial dose being administered once, wherein optionally said initial dose is administered on day 1, said elevated dose is administered on day 8 and the said further dose is administered on day 15 of the step-in cycle.

In preferred embodiments, said initial dose is about 10 μg/kg of the subject's body weight;
said elevated dose is about 30 μg/kg of the subject's body weight; and
said further dose is about is 90 μg/kg of the subject's body weight.

In certain embodiments, the method further comprises a first treatment cycle following the step-in cycle, said first treatment cycle comprising the steps of:
administering to a subject a first target dose of said T-cell engaging protein, said first target dose ranging from about 80 μg/kg of the subject's body weight to about 1600 μg/kg of the subject's body weight,
administering to the subject a second target dose of the T-cell engaging protein, said second target dose ranging from about 80 μg/kg of the subject's body weight to about 1600 μg/kg of the subject's body weight, and
administering to the subject a third target dose of the T-cell engaging protein, said third target dose ranging from about 80 μg/kg of the subject's body weight to about 1600 μg/kg of the subject's bodyweight, wherein optionally the amount of all three target doses is identical. Preferably, at least one of the first, second and third target doses is about 90 μg/kg, about 180 μg/kg, about 270 μg/kg, about 360 μg/kg, about 540 μg/kg, about 720 μg/kg, about 1080 μg/kg or about 1530 μg/kg of the subject's body weight. More preferably, said first, second and third target doses are identical about 90 μg/kg, about 180 μg/kg, about 360 μg/kg, about 540 μg/kg, about 720 μg/kg, about 1080 μg/kg or about 1530 μg/kg of the subject's body weight. More preferably, the target dose is about 1080 μg/kg or about 1530 μg/kg of the subject's body weight.

In certain embodiments, the method further comprises a first treatment cycle following the step-in cycle, the first treatment cycle comprising the steps of:
administering to a subject a first target dose of the T-cell engaging protein, said first target dose ranging from about 5 mg to about 70 mg,
administering to the subject a second target dose of the T-cell engaging protein, said second target dose ranging from about 5 mg to about 70 mg, and
administering to the subject a third target dose of the T-cell engaging protein, said third dose ranging from about 5 mg to about 70 mg,
   wherein optionally the amount of all three target doses is identical.

Preferably, at least one of the first, second or third target doses of the first treatment cycle comprises more than 30 mg.

Preferably, at least one of said first, second or third target doses of the first treatment cycle is about 5 mg, about 10 mg, about 20 mg, about 30 mg, about 40 mg, about 50 mg, about 60 mg, or about 70 mg, optionally about 10 mg or about 60 mg.

In a more preferred embodiment, the amounts of all three target doses are identical and about 10 mg. In case of safety concerns, said amount may be further reduced and be in the range of about 5 mg to about 10 mg, and is in particular about 5 mg.

In another more preferred embodiment, the amounts of all three target doses are identical and about 60 mg.

In case of safety concerns, said amount may be further reduced and be in the range of about 20 mg to about 60 mg, and is in particular about 30 mg.

In more preferred embodiments, said initial dose is about 10 μg/kg of the subject's body weight; said elevated dose is about 30 μg/kg of the subject's body weight; and said further dose is about is 90 μg/kg of the subject's body weight, and the amounts of all three target doses are identical and about 10 mg or about 60 mg.

In preferred embodiments, said first, second and third target doses are administered within three weeks of the first treatment cycle, wherein optionally said first target dose is administered on day 1, said second target dose is administered on day 8 and said third target dose is administered on day 15 of said first treatment cycle, wherein optionally said first treatment cycle has a duration of three weeks.

In preferred embodiments, said first treatment cycle is followed by a second treatment cycle, said second treatment cycle comprising the step of or consisting of the step:
administering to the subject a target dose of said T-cell engaging protein, said target dose ranging from about 80 μg/kg of the subject's body weight to about 1600 μg/kg of the subject's body weight;
   Wherein said second treatment cycle is optionally repeated at least once. Preferably, the target dose is about 90 μg/kg, about 180 μg/kg, about 270 μg/kg, about 360 μg/kg, about 540 μg/kg, about 720 μg/kg, about 1080 μg/kg or about 1530 μg/kg of the subject's body weight. More preferably, said target dose is about 90 μg/kg, about 180 μg/kg, about 270 μg/kg, about 360 μg/kg, about 540 μg/kg, about 720 μg/kg, about 1080 μg/kg or about 1530 μg/kg of the subject's body weight.

In preferred embodiments, said first treatment cycle is followed by a second treatment cycle, said second treatment cycle comprising the step of or consisting of the step:
administering to the subject a target dose of said T-cell engaging protein, said target dose ranging from about 5 mg to about 70 mg;
   wherein said second treatment cycle is optionally repeated at least once.

Preferably, said second treatment cycle has a total duration of three weeks and/or said target dose is administered on day 1 of the three weeks. Preferably, said target dose is about 10 mg or about 60 mg. In case of safety concerns, said dose of about 60 mg may be further reduced and be in the range of about 20 mg to about 60 mg, and is in particular about 30 mg. Alternatively, in said dose of about 10 mg may be further reduced and be in the range of about 5 mg to about 10 mg, and is in particular about 5 mg.

In certain embodiments, the first treatment cycle directly follows said step-in cycle; and/or the second treatment cycle directly follows said first treatment cycle.

In some embodiments, essentially no cytokine release syndrome occurs after said elevated dose of said step-in cycle in the subject.

In some embodiments, said T-cell engaging protein is the only anti-cancer agent to be administered.

In some embodiments, at least one further anti-cancer agent is to be administered, said further anti-cancer agent preferably being a chemotherapeutic agent.

In certain embodiments, the subject has been treated with a chemotherapy involving administration of inhibitors of cell growth and/or division prior to the step-in cycle.

In certain embodiments, an occurrence of cytokine release syndrome is to be treated with an anti-cytokine release syndrome agent, preferably selected from the group consisting of a corticosteroid, an IL-6 antagonist and an IL-6R antagonist.

In certain embodiments, a treatment regimen may be as follows:

TABLE 1B
Embodiment of the method of treatment according to the present invention.
Regimen B3:
one cycle = 3 weeks duration
Step-in cycle as defined below
One first treatment cycle: Target doses administered weekly
Second treatment cycles: Target doses administered every three weeks
until progression or toxicity, preferably for a maximum of 36 months

| | | | | | | | second |
|---|---|---|---|---|---|---|---|
| | | | | | | | treatment |
| | | | | | | | cycles |
| | step-in cycle | | | first treatment cycle | | | (repeated) |
| | d 1 | d 8 | d 15 | d 1 | d 8 | d 15 | d 1 |
| dose | 10 μg/kg | 30 μg/kg | 90 μg/kg | Target dose: | Target dose: | Target dose: | Target dose: |
| | | | | 1080 and | 1080 and | 1080 and | from 1080 and |
| | | | | 1530 μg/kg | 1530 μg/kg | 1530 μg/kg | 1530 μg/kg |
| | | | | | | | d 1 only |

### Dosing Regimens B4A-B4D

According to the second aspect, the present invention may relate to a T-cell engaging protein comprising a first antigen binding unit that specifically binds to DLL3 and a second antigen binding unit that specifically binds to CD3 for use in a method of treatment of a DLL3-positive cancer or a cancer selected from a Neuroendocrine Neoplasm (NEN), Merkel cell carcinoma (MCC), medullary thyroidal carcinoma and glioma,
the method comprising a step-in cycle, said step-in cycle comprising the steps of:
   administering to a subject an initial dose of said T-cell engaging protein, said initial dose ranging from about 5 μg/kg of the subject's body weight to about 15 μg/kg of the subject's body weight,
   administering to the subject an elevated dose, which is elevated as compared to said initial dose, of said T-cell engaging protein, the elevated dose ranging from about 15 μg/kg of the subject's body weight to about 100 μg/kg of the subject's body weight, or the elevated dose ranging from about 5 mg to about 20 mg, and
   administering to the subject a further dose of the T-cell engaging protein, said further dose ranging from about 5 mg to about 70 mg.

The preferred embodiments of the third aspect of the invention set forth under the sub-section “Dosing Regimen B2” apply mutatis mutandis to the second aspect and the dosing regimens B4A-B4B. They will not repeated for the sake of conciseness.

### Dosing Regimen B4A

In some embodiments of the second aspect, the present invention relates to a first antigen binding unit that specifically binds to DLL3 and a second antigen binding unit that specifically binds to CD3 for use in a method of treatment of a DLL3-positive cancer or a cancer selected from a Neuroendocrine Neoplasm (NEN), Merkel cell carcinoma (MCC), medullary thyroidal carcinoma and glioma,
the method comprising a step-in cycle, said step-in cycle comprising the steps of:
   administering to a subject an initial dose of said T-cell engaging protein, said initial dose ranging from about 5 μg/kg of the subject's body weight to about 15 μg/kg of the subject's body weight,
   administering to the subject an elevated dose, which is elevated as compared to said initial dose, of said T-cell engaging protein, the elevated dose ranging from about 60 μg/kg of the subject's body weight to about 100 μg/kg of the subject's body weight, and
   administering to the subject a further dose of the T-cell engaging protein, said further dose ranging from about 5 mg to about 20 mg, preferably from about 5 mg to about 10 mg.

In a preferred embodiment, said initial dose is about 10 μg/kg, said elevated dose is about 90 μg/kg and said further dose is about 10 mg. Alternatively, said further dose ranges from about 5 mg to about 10 mg. In case of safety concerns, said elevated dose and/or said further dose may be further reduced and be in the range of about 5 mg to about 10 mg, and is in particular about 5 mg. The dose same reduction may then apply for the dose in the first, second and/or any subsequent treatment cycle.

TABLE 1Ba
Embodiment of the method according to the present invention:
Regimen B4a:
one cycle = 3 weeks duration
Step-in cycle: initial 3 doses on weekly intervals as “step-in”
Treatment cycle 1: Target dose 10 mg administered weekly
Treatment cycles 2 (repeated): Target dose 10 mg administered once
every 3 weeks (q3w) until progression or toxicity, maximum of 36 months

| | | | | | | | Treatment |
|---|---|---|---|---|---|---|---|
| | | | | | | | cycles 2 |
| | Step-in cycle | | | Treatment cycle 1 | | | (repeated) |
| | d 1 | d 8 | d 15 | d 1 | d 8 | d 15 | d 1 |
| dose | 10 μg/kg | 90 μg/kg | 10 mg | Target dose: | Target dose: | Target dose: | Target dose: |
| | | | | 10 mg | 10 mg | 10 mg | 10 mg |

### Dosing Regimen B4B

In some embodiments of the second aspect, the present invention relates to a first antigen binding unit that specifically binds to DLL3 and a second antigen binding unit that specifically binds to CD3 for use in a method of treatment of a DLL3-positive cancer or a cancer selected from a Neuroendocrine Neoplasm (NEN), Merkel cell carcinoma (MCC), medullary thyroidal carcinoma and glioma, the method comprising a step-in cycle, said step-in cycle comprising the steps of:
administering to a subject an initial dose of said T-cell engaging protein, said initial dose ranging from about 5 μg/kg of the subject's body weight to about 15 μg/kg of the subject's body weight,
administering to the subject an elevated dose, which is elevated as compared to said initial dose, of said T-cell engaging protein, the elevated dose ranging from about 5 mg to about 20 mg, and
administering to the subject a further dose of the T-cell engaging protein, said further dose ranging from about 50 mg to about 70 mg.

In a preferred embodiment, said initial dose is about 10 μg/kg, said elevated dose is about 10 mg and said further dose is about 60 mg. In case of safety concerns, said elevated dose may be further reduced and be in the range of about 5 mg to about 10 mg, and is in particular about 5 mg. In addition, in case of safety concerns, said further dose might be further reduced and in the range of about 20 mg to about 60 mg, and is in particular about 30 mg. The dose same reduction may then apply for the dose in the first, second and/or any subsequent treatment cycle.

TABLE 1Bb
Embodiment of the method according to the present invention:
Regimen B4b:
one cycle = 3 weeks duration
Step-in cycle: initial 3 doses on weekly intervals as “step-in”
Treatment cycle 1: Target dose 60 mg administered weekly
Treatment cycles 2 (repeated): Target dose 60 mg administered once
every 3 weeks (q3w) until progression or toxicity, maximum of 36 months

| | | | | | | | Treatment |
|---|---|---|---|---|---|---|---|
| | | | | | | | cycles 2 |
| | Step-in cycle | | | Treatment cycle 1 | | | (repeated) |
| | d 1 | d 8 | d 15 | d 1 | d 8 | d 15 | d 1 |
| dose | 10 μg/kg | 10 mg | 60 mg | Target dose: | Target dose: | Target dose: | Target dose: |
| | | | | 60 mg | 60 mg | 60 mg | 60 mg |

### Dosing Regimen B4C

In some embodiments of the second aspect, the present invention relates to a first antigen binding unit that specifically binds to DLL3 and a second antigen binding unit that specifically binds to CD3 for use in a method of treatment of a DLL3-positive cancer or a cancer selected from a Neuroendocrine Neoplasm (NEN), Merkel cell carcinoma (MCC), medullary thyroidal carcinoma and glioma,
the method comprising a step-in cycle, said step-in cycle comprising the steps of:
   administering to a subject an initial dose of said T-cell engaging protein, said initial dose ranging from about 5 μg/kg of the subject's body weight to about 15 μg/kg of the subject's body weight,
   administering to the subject an elevated dose, which is elevated as compared to said initial dose, of said T-cell engaging protein, the elevated dose ranging from about 50 μg/kg of the subject's body weight to about 80 μg/kg of the subject's body weight, and
   administering to the subject a further dose of the T-cell engaging protein, said further dose ranging from about 5 mg to about 20 mg.

In a preferred embodiment, said initial dose is about 10 μg/kg, said elevated dose is about 60 μg/kg and said further dose is about 10 mg. In case of safety concerns, said further dose may be further reduced and be in the range of about 5 mg to about 10 mg, and is in particular about 5 mg. The dose same reduction may then apply for the dose in the first, second and/or any subsequent treatment cycle.

TABLE 1Bc
Embodiment of the method according to the present invention:
Regimen B4c:
one cycle = 3 weeks duration
Step-in cycle: initial 3 doses on weekly intervals as “step-in”
Treatment cycle 1: Target dose 10 mg administered weekly
Treatment cycles 2 (repeated): Target dose 10 mg administered once every
3 weeks (q3w) until progression or toxicity, maximum of 36 months

| | | | | | | | Treatment |
|---|---|---|---|---|---|---|---|
| | | | | | | | cycles 2 |
| | Step-in cycle | | | Treatment cycle 1 | | | (repeated) |
| | d 1 | d 8 | d 15 | d 1 | d 8 | d 15 | d 1 |
| dose | 10 μg/kg | 60 μg/kg | 10 mg | Target dose: | Target dose: | Target dose: | Target dose: |
| | | | | 10 mg | 10 mg | 10 mg | 10 mg |

### Dosing Regimen B4D

In some embodiments of the second aspect, the present invention relates to a first antigen binding unit that specifically binds to DLL3 and a second antigen binding unit that specifically binds to CD3 for use in a method of treatment of a DLL3-positive cancer or a cancer selected from a Neuroendocrine Neoplasm (NEN), Merkel cell carcinoma (MCC), medullary thyroidal carcinoma and glioma,
the method comprising a step-in cycle, said step-in cycle comprising the steps of:
   administering to a subject an initial dose of said T-cell engaging protein, said initial dose ranging from about 5 μg/kg of the subject's body weight to about 15 μg/kg of the subject's body weight,
   administering to the subject an elevated dose, which is elevated as compared to said initial dose, of said T-cell engaging protein, the elevated dose ranging from about 50 μg/kg of the subject's body weight to about 80 μg/kg of the subject's body weight, and
   administering to the subject a further dose of the T-cell engaging protein, said further dose ranging from about 50 mg to about 70 mg.

In a preferred embodiment, said initial dose is about 10 μg/kg, said elevated dose is about 60 μg/kg and said further dose is about 60 mg. In case of safety concerns, said amount may be further reduced and be in the range of about 20 mg to about 60 mg, and is in particular about 30 mg. The dose same reduction may then apply for the dose in the first, second and/or any subsequent treatment cycle.

TABLE 1Bd
Embodiment of the method according to the present invention:
Regimen B4d:
one cycle = 3 weeks duration
Step-in cycle: initial 3 doses on weekly intervals as “step-in”
Treatment cycle 1: Target dose 10 mg administered weekly
Treatment cycles 2 (repeated): Target dose 10 mg administered once every
3 weeks (q3w) until progression or toxicity, maximum of 36 months

| | | | | | | | Treatment |
|---|---|---|---|---|---|---|---|
| | | | | | | | cycles 2 |
| | Step-in cycle | | | Treatment cycle 1 | | | (repeated) |
| | d 1 | d 8 | d 15 | d 1 | d 8 | d 15 | d 1 |
| dose | 10 μg/kg | 60 μg/kg | 60 mg | Target dose: | Target dose: | Target dose: | Target dose: |
| | | | | 60 mg | 60 mg | 60 mg | 60 mg |

### Dosing Regimen Dareon™-5

According to the fifth aspect, the present invention may alternatively relate to a T-cell engaging protein comprising a first antigen binding unit that specifically binds to DLL3 and a second antigen binding unit that specifically binds to CD3 for use in a method of treatment of a DLL3-positive cancer or a cancer selected from a Neuroendocrine Neoplasm (NEN), Merkel cell carcinoma (MCC), medullary thyroidal carcinoma and glioma,
the method comprising a step-in cycle, the step-in cycle comprising the steps of:
   administering to a subject an initial dose of said T-cell engaging protein, said initial dose ranging from about 5 μg/kg of the subject's body weight to about 15 μg/kg of the subject's body weight,
   administering to the subject an elevated dose, which is elevated as compared to said initial dose, of said T-cell engaging protein, the elevated dose ranging from about 15 μg/kg of the subject's body weight to about 40 μg/kg of the subject's body weight,
   administering to the subject a further dose of said T-cell engaging protein, said further dose ranging from about 80 μg/kg of the subject's body weight to about 1600 μg/kg of the subject's bodyweight.

The explanations in the preceding sub-sections “Dosing Regimen B2, Dosing Regimen B3, Dosing Regimens B4a-B4d and the preferred embodiments disclosed therein equally apply to the fifth aspect.

A preferred initial dose is between about 5 and about 15 μg/kg such as about 10 μg/kg.

A preferred elevated dose is between about 20 and 40 μg/kg such as about 30 μg/kg.

In other preferred embodiments, said further dose is about 90 μg/kg. To the extent these values are lower than preferred target doses to be administered in the first treatment cycle as specified below, these lower values may be applied dependent on the clinical symptoms of the patient and/or serve to avoid the onset of severe side effects such as CRS and ICANS, severity preferably being defined in terms of the respective scoring schemes as defined further below.

In a more preferred embodiment, the initial dose is about 10 μg/kg, the elevated dose is about 30 μg/kg and the further dose is about 90 μg/kg of the subject's body weight.

It is understood that any of these specific values, in addition to being preferred specific values, also define upper and/or lower boundaries of preferred sub-intervals. This applies throughout this disclosure.

As such said further dose is preferably between about 80 and about 1530 μg/kg including between about 180 μg/kg and about 1080 μg/kg or between about 360 and about 720 μg/kg.

In those cases where treatment tolerability is good, said initial dose is given once, said elevated dose is also given once, and said further dose, also to be given once, is a preferred target dose as defined in the context of the first treatment cycle.

On the other hand, and as noted above, it is a contribution made by the present invention to accommodate more complicated situations where initial and/or elevated dose are not well tolerated. For more details in that respect, reference is made to the section entitled “Contributions made by the present invention” herein above.

Accordingly, in further embodiments, said initial dose, said elevated dose and/or said further dose is/are administered once; or said initial dose, said elevated dose and/or said further dose is/are administered more than once, optionally 2 to 10 times, wherein optionally the number of administrations is determined based on presence and/or degree of a cytokine release syndrome (CRS) and/or of an immune effector cell-associated neurotoxicity syndrome (ICANS), and wherein further optionally said elevated dose follows the initial dose only if said CRS, to the extent determined, is rated grade 1 or less in terms of NCI CTC grade and/or if said ICANS, to the extend determined, is rated grade 1 or less, and/or wherein further optionally further optionally said further dose follows the elevated dose only if said CRS, to the extent determined, is rated grade 1 or less in terms of NCI CTC grade and/or if said ICANS, to the extend determined, is rated grade 1 or less. In other words, said initial dose may be administered more than once, optionally 2 to 10 times, in view of the above NCI CTC or ICANS criteria. This means that the initial dose may be repeated before an elevated dose can be administered. Preferably, such repetition is done once in a week. The administrations and repetitions may be spaced apart a week. More preferably, said initial dose may be repeated 2, 3, 4, 5, 6, 7, 8, 9 or 10 times. In addition or alternatively, the same also applies for the elevated dose before the further dose is administered within the step-in cycle. Yet further, also said further dose may be repeated, e.g., 2, 3, 4, 5, 6, 7, 8, 9 or 10 times where deemed necessary in view of adverse responses.

Moreover, in case adverse responses occur, alternatively or in addition to repeating a dose (initial dose or elevated dose), a given dose may be reduced as compared to the preceding initial or elevated dose, e.g., to about 10% to about 90% of the preceding dose such about ⅔ or about ⅓, preferably about 50% of the preceding dose. Of note, the option of dose reduction does not only apply to the step-in cycle, but also to any of the treatment cycles disclosed herein.

In preferred embodiments, said initial dose is administered three times to ten times within the step-in cycle, preferably 3, 4, 5, 6, 7, 8, 9 or 10 times. Alternatively or in addition, the same also applies to the elevated dose.

In preferred embodiments, the administration of the doses may be spaced one week apart.

In preferred embodiments, said initial dose is administered three times within three weeks, optionally on day 1, day 8 and day 15 of the step-in cycle, if it is administered for three times. For example, the elevated dose may then be administered on day 21 of the step-in cycle, if the above NCI CTC and/or ICANS criteria are fulfilled. If the initial dose is administered 2 times, it may be administered on day 1 and day 8 in the step-in cycle, wherein the elevated dose follows in day 15. The same considerations apply to the elevated dose. If, after administration of the elevated dose, the NCI CTC and/or ICANS criteria are ful-filled, the further dose is administered next.

In some embodiments, said step-in cycle has a duration of at least three weeks. In preferred embodiments, said step-in cycle may have a duration of three weeks, wherein optionally the initial dose may be administered on day 1, the elevated dose may be administered on day 8 and the further dose may be administered on day 15. Depending on how many times the initial and/or the first dose may be administered, the step-in cycle may have a longer duration of than three weeks.

In some embodiments, said initial, elevated and further doses are administered within three weeks, said initial dose being administered once, wherein optionally said initial dose is administered on day 1, said elevated dose is administered on day 8 and said further dose is administered on day 15 of said step-in cycle.

To the extent reference is made to specific days such as day 1, day 8, day 15 etc. it is to be understood that these are particularly preferred days which, however, allow for certain deviations depending on the patient's compliance or availability as well as the attending clinician's capacities. In other words, administrations on, say, days 1, 9 and 14 are within the scope of the present invention. Generally speaking, it will be sufficient to administer once per week regardless of the specific day within a given week. More preferably, deviations from the most preferred scheme (days 1, 8, 15 etc.) should not exceed three, two or one days in either direction.

Of note, the dependence of a given dose on the patient's body weight during the step-in cycle confers distinct advantages. In particular, such dosing provides improved tolerability including reduced frequency and/or severity of CRS, and furthermore allows for higher target doses in the course of treatment cycles. Without wishing to be bound by a specific theory, it is considered that these advantageous effects results from a controlled cytokine depletion during the step-in cycle.

In accordance with the above, in preferred embodiments, the method further comprises a first treatment cycle following said step-in cycle, said first treatment cycle comprising the steps of:
administering to a subject a first target dose of the T-cell engaging protein, said first target dose ranging from about 5 mg to about 70 mg,
administering to the subject a second dose of the T-cell engaging protein, said second dose ranging from about 5 mg to about 70 mg, and
administering to the subject a third dose of the T-cell engaging protein, said third dose ranging from about 5 mg to about 70 mg,
   wherein optionally the amount of all three target doses is identical.

In some embodiments, at least one of said first, second or third target doses of said first treatment cycle comprises more than 30 mg.

In some embodiments, at least one of said first, second or third target doses of the first treatment cycle is about 5 mg, about 10 mg, about 20 mg, about 30 mg, about 40 mg, about 50 mg, about 60 mg, or about 70 mg, optionally about 10 mg or about 60 mg.

In preferred embodiments, the amount of all three target doses are identical and about 10 mg. In case of safety concerns, said amount may be further reduced and be in the range of about 5 mg to about 10 mg, and in particular is about 5 mg. The same may then apply to the dose of any subsequent treatment cycles.

In alternative preferred embodiments, the amount of all three target doses are identical and about 60 mg. In case of safety concerns, said amount may be further reduced and be in the range of about 20 mg to about 60 mg, and in particular is about 30 mg. The same may then apply to the dose of any subsequent treatment cycles.

In preferred embodiments, said first treatment cycle is not repeated or said first treatment cycle is repeated at least once. Preferred numbers of repetitions are disclosed herein and exemplified in the Examples.

In preferred embodiments, said first, second and third target doses are administered within three weeks of said first treatment cycle, wherein optionally said first target dose is administered on day 1, the second target dose is administered on day 8 and the third target dose is administered on day 15 of said first treatment cycle, wherein optionally said first treatment cycle has a duration of three weeks. Reference is made to a certain flexibility regarding intervals or specific treatment days as explained further above in the context of the description of the step-in cycle.

In preferred embodiments, said the first treatment cycle is followed by a second treatment cycle, said second treatment cycle comprising the step of or consisting of the step:
administering to the subject a target dose of the T-cell engaging protein, said target dose ranging from about 5 mg to about 70 mg;
   wherein said second treatment cycle is optionally repeated at least once. Preferably, said target dose is about 10 mg or about 60 mg. In case of safety concerns, said target dose of about 10 mg may be further reduced and be in the range of about 5 mg to about 10 mg, and in particular is about 5 mg. The same may then apply to the dose of any subsequent treatment cycles. In case of safety concerns, said target dose of about 60 mg may be further reduced and be in the range of about 20 mg to about 60 mg, and in particular is about 30 mg.

The same may then apply to the dose of any subsequent treatment cycles.

In preferred embodiments, said second treatment cycle has a total duration of three weeks and/or said target dose is administered on day 1 of said three weeks. Reference is made to optional or preferred repetitions of treatments cycle as disclosed herein.

In other words, if a patient is considered to be in need of further treatment after the first treatment cycle, the burden on the patient can be reduced by not requiring any more that the patient presents itself once a week in the clinic, but in larger intervals, a preferred larger interval including, but not being confined to three weeks as is subject of the preferred embodiment above. Alternatively, also intervals of about two weeks or every ten or every 20 days may be considered.

In preferred embodiments, said first treatment cycle directly follows said step-in cycle; and/or said second treatment cycle directly follows said first treatment cycle. To explain further, a dosage regimen comprising step-in cycle as well as first and second treatment cycle may involve administrations on days 1, 8, 15, 22, 29, 36, 43, 64, 85 etc., wherein days 1, 8 and 15 would correspond to the step-in cycle (step-in doses being tolerable in this example such that no repetitions are required), days 22, 29, 36 (counting continued from the day of administration of the first dose of the step-in cycle) would correspond to one application of the first treatment cycle (weekly target dose), and days 43, 64, 85 etc. would correspond to repeated (here three) applications of the second treatment cycle (3 week administration intervals in this example).

In some embodiments, essentially no cytokine release syndrome occurs after said elevated dose of said step-in cycle in the subject.

This would generally be scenario where the patient under consideration would qualify for said further dose of said step-in cycle being the same as a preferred target dose of said first treatment cycle. Optionally, premedication may be adjusted under such circumstances as disclosed herein.

In some embodiments, said T-cell engaging protein is the only anti-cancer agent to be administered.

In some embodiments, at least one further anti-cancer agent is to be administered, said further anti-cancer agent preferably being a chemotherapeutic agent. Preferred chemotherapeutic agents are those disclosed in the background section herein above which is part of the disclosure of the invention as well as those referred to in the Examples.

In certain embodiments, the subject has been treated with a chemotherapy involving administration of inhibitors of cell growth and/or division prior to the step-in cycle.

In certain embodiments, an occurrence of cytokine release syndrome is to be treated with an anti-cytokine release syndrome agent, preferably selected from the group consisting of a corticosteroid, an IL-6 antagonist and an IL-6R antagonist.

Overall, preference is given to treatment cycles of 3 weeks duration.

In certain embodiments, a treatment regimen may be as follows:

TABLE 1C
Embodiment of the method of treatment according to the present invention.
Regimen Dareon ™-5:
1 cycle = 3 weeks duration
Step-in cycle as defined below
One first treatment cycle: Target doses (x mg) administered weekly
Second treatment cycles: Target doses administered every 3 weeks
until progression or toxicity, preferably a maximum of 36 months

| | | | | | | | second |
|---|---|---|---|---|---|---|---|
| | | | | | | | treatment |
| | | | | | | | cycles |
| | step-in cycle | | | first treatment cycle | | | (repeated) |
| | d 1 | d 8 | d 15 | d 1 | d 8 | d 15 | d 1 |
| dose | 10 μg/kg | 30 μg/kg | 90 μg/kg | Target dose: | Target dose: | Target dose: | Target dose: |
| | | | | From 10 mg | From 10 mg | From 10 mg | From 10 mg to |
| | | | | to 60 mg | to 60 mg | to 60 mg | 60 mg d1 only |

Of note, preferred target doses are between about 10 and about 60 mg. Optionally, and/or where deemed necessary by the attending clinician, said target doses may be reduced, for example by 50%, i.e., to about 5 mg to about 30 mg. For example, if there is limited tolerability in response to a target dose of 60 mg, a preferred next target dose to be administered would be 30 mg.

### Mode of Administration (Applies to all Regimens Disclosed Herein)

An administration of the T-cell engaging protein disclosed herein within an regimen according to the present invention may be carried out in various ways.

The T-cell engaging protein within the regimens disclosed herein may be administered to the subjects by any suitable means, selected from the group consisting of parenteral, subcutaneous, intraperitoneal, intrapulmonary, intranasal, and/or intralesional administration. Parenteral administration includes administrations selected from the group consisting intramuscular, intravenous, intraarterial, intraperitoneal, or subcutaneous administration. In preferred embodiments, the T-cell engaging protein is administered by intravenous (IV) infusion.

### Formulation

Pharmaceutical preparations for parenteral administration, may for example be sterile solutions, suspensions, dispersions, emulsions, or powders which comprise the active ingredient and which are suitable, optionally after a further dissolution or dilution step, for infusion or injection. Suitable carriers or diluents for such preparations for example include, without limitation, sterile water and pharmaceutically acceptable aqueous buffers and solutions such as physiological phosphate-buffered saline, Ringer's solutions, dextrose solution, and Hank's solution; water oils; glycerol; ethanol; glycols such as propylene glycol, as well as mineral oils, animal oils and vegetable oils, for example peanut oil, soybean oil, as well as suitable mixtures thereof.

Solutions of the T cell engaging proteins disclosed herein may also contain a preservative to prevent the growth of microorganisms, such as antibacterial and antifungal agents, for example, p-hydroxybenzoates, parabens, chlorobutanol, phenol, sorbic acid, thiomersal, (alkali metal salts of) ethylenediamine tetraacetic acid, and the like. In many cases, it will be preferable to include isotonic agents, for example, sugars, buffers or sodium chloride. Optionally, emulsifiers and/or dispersants may be used. The proper fluidity can be maintained, for example, by the formation of liposomes, by the maintenance of the required particle size in the case of dispersions or by the use of surfactants. Other agents delaying absorption, for example, aluminum monostearate and gelatin, may also be added. The solutions may be filled into injection vials, ampoules, infusion bottles, and the like.

In all cases, the ultimate dosage form must be sterile, fluid and stable under the conditions of manufacture and storage. Sterile injectable solutions are prepared by incorporating the active compound in the required amount (e.g. 10 mg/ml to 50 mg/ml, preferably any one of 10, 20, 30, 40 or 50 mg/ml) in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

Generally, suitable formulations for therapeutic proteins such as the T cell engaging proteins disclosed herein are buffered protein solutions, such as solutions including the protein in a suitable concentration and an aqueous buffer such as:
phosphate buffered saline, pH 7.4,
other phosphate buffers, pH 6.2 to 8.2,
acetate buffers, pH 3.2 to 7.5, preferably pH 4.8 to 5.5
histidine buffers, pH 5.5 to 7.0,
succinate buffers, pH 3.2 to 6.6, and
citrate buffers, pH 2.1 to 6.2,
   and, optionally, salts (e.g. NaCl) and/or sugars (such as e.g. sucrose and trehalose) and/or other polyalcohols (such as e.g. mannitol and glycerol) for providing isotonicity of the solution.

In addition, other agents such as a detergent, e.g. 0.02% Tween-20 or Tween-80, may be included in such solutions.

The above described formulations can optionally be provided as lyophilized formulation that is to be reconstituted in a solution, e.g. in water for injection (WFI).

It will be clear to the person skilled in the art that the ingredients and the amounts thereof as given above do only represent one, preferred option. Alternatives and variations thereof will be immediately apparent to the skilled person, or can easily be conceived starting from the above disclosure.

The T-cell engaging protein is preferably formulated in an aqueous solution or as lyophilisate, preferably buffered, and optionally comprising one, more or all of the following: a mono- or disaccharide, methionine, and a surfactant or detergent. A preferred pH value is between about 5 and about 7 such as about 6. Preferred buffer substances include histidine, citrate and acetate. Preferred mono- and disaccharides include trehalose and sucrose (e.g. between 160 to 260 mM). A preferred surfactant is polysorbate 20 (PS20).

Particularly preferred formulations for the T cell engaging proteins (e.g. at a concentration of any one of 10, 20, 30, 40 or 50 mg/ml) are:
10 mM histidine, 240 mM trehalose, optionally 10 mM methionine, and 0.4 mg/ml PS20; or
10 mM histidine, 240 mM sucrose, and 0.4 mg/ml PS20;
in either case adjusted to a pH of about 6.0.

### Method of Treatment and Uses for the Manufacture of a Medicament

The aspects of the present invention and the corresponding embodiments can also be understood in terms of a method of treatment.

Accordingly, one aspect of the present invention provides a method of treatment of a DLL3-positive cancer or a cancer selected from a Neuroendocrine Neoplasm (NEN), Merkel cell carcinoma (MCC), medullary thyroidal carcinoma and glioma,
the method comprising a step-in cycle, said step-in cycle comprising the steps of:
   administering to a subject an initial dose of said T-cell engaging protein, said initial dose ranging from about 5 μg/kg of the subject's body weight to about 40 μg/kg of the subject's body weight,
   administering to the subject an elevated dose, which is elevated as compared to said initial dose, of said T-cell engaging protein, the elevated dose ranging from about 15 μg/kg of the subject's body weight to about 100 μg/kg of the subject's body weight, or the elevated dose ranging from about 5 mg to about 20 mg, and
   administering to the subject a further dose of the T-cell engaging protein, said further dose ranging from about 80 μg/kg of the subject's body weight to about 1600 μg/kg of the subject's bodyweight, wherein said further dose is equal to or higher than said elevated dose, or said further dose ranging from about 5 mg to about 70 mg.

In a further aspect, the present invention provides a method of treatment of a DLL3-positive cancer or a cancer selected from a Neuroendocrine Neoplasm (NEN), Merkel cell carcinoma (MCC), medullary thyroidal carcinoma and glioma,
the method comprising a step-in cycle, said step-in cycle comprising the steps of:
   administering to a subject an initial dose of said T-cell engaging protein, said initial dose ranging from about 5 μg/kg of the subject's body weight to about 15 μg/kg of the subject's body weight,
   administering to the subject an elevated dose, which is elevated as compared to said initial dose, of said T-cell engaging protein, the elevated dose ranging from about 20 μg/kg of the subject's body weight to about 100 μg/kg of the subject's body weight, or the elevated dose ranging from about 5 mg to about 20 mg, and
   administering to the subject a further dose of the T-cell engaging protein, said further dose ranging from about 5 mg to about 70 mg.

In a further aspect, the present invention provides a method of treatment of a DLL3-positive cancer or a cancer selected from a Neuroendocrine Neoplasm (NEN), Merkel cell carcinoma (MCC), medullary thyroidal carcinoma and glioma,
the method comprising a step-in cycle, said step-in cycle comprising the steps of:
   administering to a subject an initial dose of said T-cell engaging protein, said initial dose ranging from about 20 μg/kg of the subject's body weight to about 40 μg/kg of the subject's body weight,
   administering to the subject an elevated dose, which is elevated as compared to said initial dose, of said T-cell engaging protein, the elevated dose ranging from about 60 μg/kg of the subject's body weight to about 100 μg/kg of the subject's body weight,
   administering to the subject a further dose of the T-cell engaging protein, said further dose ranging from about 80 μg/kg of the subject's body weight to about 1600 μg/kg of the subject's bodyweight, wherein said further dose is equal to or higher than said elevated dose, wherein
   the T-cell engaging protein comprises a first antigen binding unit specifically binding to DLL3 and a second antigen binding unit specifically binding to CD3.

In a further aspect, the present invention provides a method of treatment of a DLL3-positive cancer or a cancer selected from a Neuroendocrine Neoplasm (NEN), Merkel cell carcinoma (MCC), medullary thyroidal carcinoma and glioma,
the method comprising a step-in cycle, the step-in cycle comprising the steps of:
   administering to the subject an initial dose, an elevated dose, which is elevated as compared to said initial dose, and a further dose of said T-cell engaging protein, wherein a ratio between said initial, elevated and further dose per kilogram of the subject's body weight is about 1: about 3: about 9, wherein
      the T-cell engaging protein comprises a first antigen binding unit specifically binding to DLL3 and a second antigen binding unit specifically binding to CD3.

In a further aspect, the present invention provides a method of treatment of a DLL3-positive cancer or a cancer selected from a Neuroendocrine Neoplasm (NEN), Merkel cell carcinoma (MCC), medullary thyroidal carcinoma and glioma,
the method comprising a step-in cycle, the step-in cycle comprising the steps of:
   administering to a subject an initial dose of said T-cell engaging protein, said initial dose ranging from about 5 μg/kg of the subject's body weight to about 15 μg/kg of the subject's body weight,
   administering to the subject an elevated dose, which is elevated as compared to said initial dose, of said T-cell engaging protein, the elevated dose ranging from about 20 μg/kg of the subject's body weight to about 40 μg/kg of the subject's body weight,
   administering to the subject a further dose of said T-cell engaging protein, said further dose ranging from about 80 μg/kg of the subject's body weight to about 1600 μg/kg of the subject's bodyweight, wherein the T-cell engaging protein comprises a first antigen binding unit specifically binding to DLL3 and a second antigen binding unit specifically binding to CD3.

In one aspect, the present invention provides a use of a T-cell engaging protein comprising a first antigen binding unit that specifically binds to DLL3 and a second antigen binding unit that specifically binds to CD3 for the manufacture of a medicament for use in a method of treatment of a DLL3-positive cancer or a cancer selected from a Neuroendocrine Neoplasm (NEN), Merkel cell carcinoma (MCC), medullary thyroidal carcinoma and glioma,
the method comprising a step-in cycle, said step-in cycle comprising the steps of:
   administering to a subject an initial dose of said T-cell engaging protein, said initial dose ranging from about 5 μg/kg of the subject's body weight to about 40 μg/kg of the subject's body weight,
   administering to the subject an elevated dose, which is elevated as compared to said initial dose, of said T-cell engaging protein, the elevated dose ranging from about 15 μg/kg of the subject's body weight to about 100 μg/kg of the subject's body weight, or the elevated dose ranging from about 5 mg to about 20 mg, and
   administering to the subject a further dose of the T-cell engaging protein, said further dose ranging from about 80 μg/kg of the subject's body weight to about 1600 μg/kg of the subject's bodyweight, wherein said further dose is equal to or higher than said elevated dose, or said further dose ranging from about 5 mg to about 70 mg.

In a further aspect, the present invention provides a use of T-cell engaging protein comprising a first antigen binding unit that specifically binds to DLL3 and a second antigen binding unit that specifically binds to CD3 for the manufacture of a medicament for use in a method of treatment of a DLL3-positive cancer or a cancer selected from a Neuroendocrine Neoplasm (NEN), Merkel cell carcinoma (MCC), medullary thyroidal carcinoma and glioma,
the method comprising a step-in cycle, said step-in cycle comprising the steps of:
   administering to a subject an initial dose of said T-cell engaging protein, said initial dose ranging from about 5 μg/kg of the subject's body weight to about 15 μg/kg of the subject's body weight,
   administering to the subject an elevated dose, which is elevated as compared to said initial dose, of said T-cell engaging protein, the elevated dose ranging from about 15 μg/kg of the subject's body weight to about 100 μg/kg of the subject's body weight, or the elevated dose ranging from about 5 mg to about 20 mg, and
   administering to the subject a further dose of the T-cell engaging protein, said further dose ranging from about 5 mg to about 70 mg.

In a further aspect, the present invention provides a use of a T-cell engaging protein comprising a first antigen binding unit specifically binding to DLL3 and a second antigen binding unit specifically binding to CD3 for the manufacture of a medicament for use in a treatment of a DLL3-positive cancer or a cancer selected from a Neuroendocrine Neoplasm (NEN), Merkel cell carcinoma (MCC), medullary thyroidal carcinoma and glioma,
the method comprising a step-in cycle, said step-in cycle comprising the steps of:
   administering to a subject an initial dose of said T-cell engaging protein, said initial dose ranging from about 20 μg/kg of the subject's body weight to about 40 μg/kg of the subject's body weight,
   administering to the subject an elevated dose, which is elevated as compared to said initial dose, of said T-cell engaging protein, the elevated dose ranging from about 60 μg/kg of the subject's body weight to about 100 μg/kg of the subject's body weight,
   administering to the subject a further dose of the T-cell engaging protein, said further dose ranging from about 80 μg/kg of the subject's body weight to about 1600 μg/kg of the subject's bodyweight, wherein said further dose is equal to or higher than said elevated dose.

In a further aspect, the present invention provides a T-cell engaging protein comprising a first antigen binding unit specifically binding to DLL3 and a second antigen binding unit specifically binding to CD3 for the manufacture of a medicament for use in a treatment of a DLL3-positive cancer or a cancer selected from a Neuroendocrine Neoplasm (NEN), Merkel cell carcinoma (MCC), medullary thyroidal carcinoma and glioma,
the method comprising a step-in cycle, the step-in cycle comprising the steps of:
   administering to the subject an initial dose, an elevated dose, which is elevated as compared to said initial dose, and a further dose of said T-cell engaging protein, wherein a ratio between said initial, elevated and further dose per kilogram of the subject's body weight is about 1: about 3: about 9.

In a further aspect, the present invention provides a T-cell engaging protein comprising a first antigen binding unit specifically binding to DLL3 and a second antigen binding unit specifically binding to CD3 for the manufacture of a medicament for use in a treatment of a DLL3-positive cancer or a cancer selected from a Neuroendocrine Neoplasm (NEN), Merkel cell carcinoma (MCC), medullary thyroidal carcinoma and glioma,
the method comprising a step-in cycle, the step-in cycle comprising the steps of:
   administering to a subject an initial dose of said T-cell engaging protein, said initial dose ranging from about 5 μg/kg of the subject's body weight to about 15 μg/kg of the subject's body weight,
   administering to the subject an elevated dose, which is elevated as compared to said initial dose, of said T-cell engaging protein, the elevated dose ranging from about 20 μg/kg of the subject's body weight to about 40 μg/kg of the subject's body weight,
   administering to the subject a further dose of said T-cell engaging protein, said further dose ranging from about 80 μg/kg of the subject's body weight to about 1600 μg/kg of the subject's bodyweight.

For brevity, the embodiments corresponding to these aspects as disclosed above, are referred to but not repeated. In particular, embodiments of the first, second and third aspect are also embodiments of the further aspects disclosed above.

### Premedication and Further Measures to Reduce Adverse Effects (AES) and Ensure Patient Safety (Applies to all Aspects of the Invention)

In certain embodiments, a premedication comprising a non-steroidal anti-inflammatory drug, such as acetaminophen, an anti-histaminicum and/or a steroid, such as prednisolone or dexamethasone, prior to at least one of the doses, optionally prior to each dose.

Premedication as such is a measure established in the art. The attending clinician, upon examination of the patient's clinical status, will be in a position to decide when a premedication is be given, when the dose of the premedication can be lowered, and when premedication can be dispensed with altogether. As a rule, at least the initial dose of the step-in cycle will be preceded by premedication. In addition, any further dose of said step-in cycle may be preceded by premedication as well. To the extent necessary, premedication may also be extended to part or all of the treatment cycles. This is exemplified in the following.

The following preferred pre-medication scheme is tailored to the specific T cell engaging protein BI 764532 but applies mutatis mutandis or can be adapted by the attending clinician without further ado to other T cell engaging proteins such as those disclosed herein or in WO 2019/234220.

In addition to determining the optimal dosing/administration regimen (see above), the tolerability of BI 764532 administration can be further improved and frequency/severity of adverse events (e.g. CRS and ICANS) reduced by administering pre-medication (treatment given prior to BI 764532 administration to prevent and/or reduce side effects).

Premedication is given 30-60 min before the start of the BI 764532 infusion. The premedication before BI 764532 administration includes:
Acetaminophen/paracetamol 1000 mg p.o. or i.v., or equivalent
Antihistamine (diphenhydramine 50 mg p.o. or i.v., or equivalent)
Steroids (dexamethasone 16 mg p.o. or i.v., or equivalent intermediate-acting corticosteroid)

If the individual patient has tolerated BI 764532 with steroid premedication without >Grade 1 IRR or CRS for at least 1 administration of target dose (i.e. through all step-in doses+target dose), steroids premedication may be reduced to 50% (e.g. steroids i.v., dexamethasone 8 mg) at start of subsequent administrations. If one BI 764532 infusion with reduced steroid premedication is tolerated without >Grade 1 IRR or CRS in an individual patient, steroid premedication should be skipped in repeated treatment cycles for the respective patient.

To further reduce the frequency and severity of adverse events (especially CRS) 1000 mL normal saline iv is administered within 1 hour after BI 764532 infusion at step-in doses and first target dose.

Hospitalization (24 h) for safety monitoring following step-in doses and first target dose, reduced safety monitoring at subsequent BI 764532 administrations if previously well tolerated.

### Indications/Patient Population

Preferred medical indications amenable to treatment with the T-cell engaging proteins of the invention are disclosed herein above. Corresponding treatment regimens are subject of the various aspects of the invention as disclosed above.

Particularly preferred indications amenable to treatment with the T cell engaging proteins of the invention are relapsed/refractory extensive-stage small cell lung cancer and other relapsed/refractory neuroendocrine carcinomas, e.g., ES-SCLC which progressed during or following at least two prior lines of treatment including platinum-based chemotherapy, ES-SCLC which progressed during or following at least one prior line of treatment, including platinum-based chemotherapy or anti PD-1/anti PD-L1 therapy, and (metastatic) epNEC which progressed during or following at least one prior line of treatment including platinum-based chemotherapy, and LCNEC of the lung, which progressed during or following at least one prior line of treatment (e.g. platinum-based regimen). In some preferred embodiments of these preferred indications, epNEC or LCNEC are locally advanced or metastatic.

Further preferred indications amenable to treatment with the T cell engaging proteins of the invention include ES-SCLC after failure of one prior line of SoC (second line, 2 L; e.g. after failure of first line (1 L) platinum based chemotherapy and/or anti-PD-L1 therapy), ES-SCLC initial treatment (first line, 1 L), LS-SCLC initial treatment (1 L), 1 L or 2 L LCNEC-Lung (after failure of SoC), 1 L epNEC, 2 L epNEC (after failure of SoC, e.g., platinum-based chemotherapy), 1 L Grade 3 NET, MCC (1 L or 2 L), glioma/glioblastoma (1 L or 2 L).

In preferred embodiments listed below, more complex treatment regimens are provided. Such more complex treatment regimens are characterized in that they may involve one, more or all of the following: (i) the established standard of care (SoC); (ii) a distinction between first line treatment (“1 L”) and second line treatment (“2 L”); and (iii) combination therapies.

In preferred embodiments, further provided herein are
combination treatments of BI 764532 according to any one of the aspects described (e.g. Dareon™-5) with platinum-based therapy (e.g., carboplatin or cisplatin)+etoposide (Dareon™-7) as first-line treatment for locally advanced or metastatic NEC (extra pulmonary or unknown primary) or LCNEC of the lung followed by maintenance treatment with BI 764532 or
combination treatments of BI 764532 according to any one of the aspects described (e.g. Dareon™-5) with platinum+etoposide+either atezolizumab or durvalumab (Dareon™-8) as first line treatment of ES-SCLC, followed by maintenance treatment with BI 764532 in combination with anti-PD-L1 (atezolizumab or durvalumab); or or
combination treatment of BI764532 according to any one of the aspects described (e.g. Dareon™-5) and topotecan for ES-SCLC that progressed or recurred following platinum-based treatment, or following anti-PD-1 or PD-L1 as applicable, and is eligible for topotecan treatment (Dareon™-9) followed by maintenance treatment with BI 764532
first line maintenance treatment after completion of chemotherapy in ES-SCLC, epNEC or LCNEC with BI764532 according to any one of the aspects described (e.g. Dareon™-5) combined with either atezolizumab or durvalumab; or
first line maintenance treatment after completion of chemotherapy in ES-SCLC, epNEC or LCNEC with BI764532 according to any one of the aspects described (e.g. Dareon™-5); or
combination of chemotherapy (e.g., platinum-based plus etoposide) and/or radiotherapy with BI 764532 according to any one of the aspects described (e.g. Dareon™-5) in 1 L LS-SCLC; or
combination of an PD-1 inhibitor (e.g. ezabenlimab) with BI 764542 according to any of the aspects described (e.g. Dareon™-5), wherein said PD-1 inhibitor may be administered on day 1 of the step-in cycle and each subsequent treatment cycle.

In another preferred embodiment, the cancer is glioma and the step-in cycle comprises the steps of:
administering to a subject an initial dose of BI 764532, said initial dose ranging from about 10 μg/kg of the subject's body weight,
administering to the subject an elevated dose, which is elevated as compared to said initial dose, of BI 764532, the elevated dose ranging from about 10 μg/kg of the subject's body weight to about 30 μg/kg of the subject's body weight, and
   administering to the subject a further dose of BI 764532, said further dose ranging from about 10 μg/kg of the subject's body weight to about 30 μg/kg of the subject's bodyweight. Any of the subsequent treatment cycles, as defined in the preceding sections herein may encompass a target dose of BI 764532 ranging from about 90 μg/kg to about 720 μg/kg, such as 90 μg/kg, 180 μg/kg, 360 μg/kg, or 720 μg/kg, of the subject's body weight or about 10 mg to about 70 mg, such as about 10 mg or about 60 mg.

In any of the preferred embodiments above, the target dose may range from about 5 mg to about 60 mg. In particular, the target dose ranges from about 5 mg to about 15 mg. The target dose may for example be about 5 mg or about 10 mg. In case of safety concerns, the target dose of about 10 mg may be further reduced and may be in the range of about 5 mg to about 10 mg, and in particular is about 5 mg. Alternatively, in case of safety concerns, said target dose of about 60 mg may be reduced and be in the range of about 20 mg to about 60 mg, and in particular is about 30 mg.

In addition or alternatively, in any of the preferred embodiments above, a treatment cycle including only chemotherapy may take place prior to any of the combination treatments or the glioma treatment described above.

In addition or alternatively, in any of the preferred embodiments above, any of the combination treatments or the glioma treatment described above may encompass 2 to 5 treatment cycles after the step-in cycle.

Of note, in the list below a number of preferred or exemplary chemotherapeutic or cytostatic agents are mentioned. Each of these agents are preferred embodiments of chemotherapeutic or cytostatic agents in accordance with the entire disclosure.
SCLC
   Extensive-stage (ES)-SCLC
      1 L (previously untreated ES-SCLC): combination with established Standard of Care (SoC), e.g., platin+etoposide+anti-PD-L1 (e.g. atezolizumab or durvalumab); maintenance treatment after completion of chemo with BI 764532+anti-PD-L1 (e.g. atezolizumab or durvalumab) or BI 764532 monotherapy
      2 L (after failure of SoC (platinum-based chemotherapy)), combination with established Standard of Care (SoC), e.g., topotecan; maintenance treatment after completion of SoC with BI 764532 mono or BI 764532+anti-PD-(L)1
      2 L and beyond (after failure of SoC (at least one line of platinum-based chemotherapy)): BI 764532 monotherapy or BI 764532+anti-PD-(L)1
   Limited-stage (LS)-SCLC
      1 L (previously untreated LS-SCLC): combination with established Standard of Care (SoC), e.g., platin+etoposide+anti-PD-L1 (e.g. atezolizumab or durvalumab); maintenance treatment after completion of chemo(radio)therapy with BI 764532+anti-PD-L1 (e.g. atezolizumab or durvalumab) or BI 764532 monotherapy
Extrapulmonary (ep)NEC including NEC with unknown primary site) advanced/metastatic/unresectable disease
   1 L (previously untreated advanced/metastatic/unresectable epNEC): combination with established Standard of Care (SoC), e.g., platin+etoposide; maintenance treatment after completion of chemo BI 764532 monotherapy or BI 764532+anti-PD-(L)1
   2 L and beyond (after failure of SoC (e.g., platinum-based chemotherapy)): BI 764532 monotherapy or BI 764532+anti-PD-(L)1
Grade 3 NET
   1 L (previously untreated advanced/metastatic/unresectable G3 NET): combination with established Standard of Care (SoC), e.g., platin+etoposide; maintenance treatment after completion of chemo with BI 764532+anti-PD-L1 (e.g. atezolizumab or durvalumab) or BI 764532 monotherapy
Large Cell Neuroendocrine Carcinoma of the Lung (LC-NEC Lung)
   1 L (previously untreated advanced/metastatic/unresectable LC-NEC lung): combination with established Standard of Care (SoC), e.g., platin+etoposide; maintenance treatment after completion of chemo BI 764532 monotherapy or BI 764532+anti-PD-(L)1
Merkel Cell Carcinoma (MCC)
   1 L (MCC, disseminated disease): combination with established Standard of Care (SoC), e.g., anti-PD1 (e.g. avelumab, nivolumab, pembrolizumab); maintenance treatment after completion of chemo BI 764532 mono or anti-PD1+BI 764532
   2 L (disseminated MCC after failure of 1^{st} line treatment): combination with established Standard of Care (SoC), e.g platin+etoposide OR BI 764532 mono; maintenance treatment after completion of chemo: BI 764532 monotherapy
Glioma/Glioblastoma
   1 L (previously untreated glioma/glioblastoma): combination with established Standard of Care (SoC), e.g., TMZ (temozolamide), radiotherapy
   2 L (recurrent or progressive glioma/glioblastoma after prior TMZ treatment): BI 764532 monotherapy or combination therapy with SoC (e.g., bevacizumab)

### Bispecific T-Cell Engaging Protein

The bispecific T-cell engaging protein for use in the method of treatments according to the present invention comprises a first antigen binding unit specifically binding to DLL3 and a second antigen binding unit specifically binding to CD3. Such T-cell engaging molecules and methods of producing the same are disclosed in WO 2019/234220 A1, the contents of which is incorporated by reference in its entirety.

In some embodiments, said bispecific T-cell engaging protein comprises immunoglobulin (Ig) domains, preferably 12 Ig domains. Optionally, said Ig domains are comprised in four polypeptide chains, wherein two light chains comprise two Ig domains each, and two heavy chains comprise four Ig domains each. Preferably, though, said Ig domains are comprised in two polypeptide chains. Advantages of only two polypeptide chains are discussed in the following as are preferred implementations of said Ig domains. For clarity, individual Ig domains are referred to as variable and constant domains of light and heavy chains, in alignment with the established nomenclature for antibodies.

In some embodiments, either antigen binding unit of said bispecific T-cell engaging protein comprises six complementarity determining regions (CDRs). Preferably, within either antigen binding unit, three CDRs are located on one Ig domain of a light chain and three CDRs are located on one Ig domain of a heavy chain. Said CDR-bearing domains are variable domains in accordance with the nomenclature used below.

In one preferred embodiment, the T-cell engaging protein of the invention comprises a first antigen binding unit specifically binding to DLL3, comprising light chain CDRs comprising the amino acid sequences of SEQ ID NO:1 (CDR1), SEQ ID NO:2 (CDR2) and SEQ ID NO:3 (CDR3) and heavy chain CDRs comprising the amino acid sequences of SEQ ID NO:4 (CDR1), SEQ ID NO:5 (CDR2) and SEQ ID NO:6 (CDR3) and a second antigen binding unit specifically binding to CD3, comprising light chain CDRs comprising the amino acid sequences of SEQ ID NO:55 (CDR1), SEQ ID NO:56 (CDR2) and SEQ ID NO:57 (CDR3) and heavy chain CDRs comprising the amino acid sequences of SEQ ID NO:58 (CDR1), SEQ ID NO:59 (CDR2) and SEQ ID NO:60 (CDR3).

In one preferred embodiment, the T-cell engaging protein of the invention comprises a first antigen binding unit specifically binding to DLL3, comprising light chain CDRs comprising the amino acid sequences of SEQ ID NO:7 (CDR1), SEQ ID NO:8 (CDR2) and SEQ ID NO:9 (CDR3) and heavy chain CDRs comprising the amino acid sequences of SEQ ID NO:10 (CDR1), SEQ ID NO:11 (CDR2) and SEQ ID NO:12 (CDR3) and a second antigen binding unit specifically binding to CD3, comprising light chain CDRs comprising the amino acid sequences of SEQ ID NO:55 (CDR1), SEQ ID NO:56 (CDR2) and SEQ ID NO:57 (CDR3) and heavy chain CDRs comprising the amino acid sequences of SEQ ID NO:58 (CDR1), SEQ ID NO:59 (CDR2) and SEQ ID NO:60 (CDR3).

In one preferred embodiment, the T-cell engaging protein of the invention comprises a first antigen binding unit specifically binding to DLL3, comprising light chain CDRs comprising the amino acid sequences of SEQ ID NO:13 (CDR1), SEQ ID NO:14 (CDR2) and SEQ ID NO:15 (CDR3) and heavy chain CDRs comprising the amino acid sequences of SEQ ID NO:16 (CDR1), SEQ ID NO:17 (CDR2) and SEQ ID NO:18 (CDR3) and a second antigen binding unit specifically binding to CD3, comprising light chain CDRs comprising the amino acid sequences of SEQ ID NO:55 (CDR1), SEQ ID NO:56 (CDR2) and SEQ ID NO:57 (CDR3) and heavy chain CDRs comprising the amino acid sequences of SEQ ID NO:58 (CDR1), SEQ ID NO:59 (CDR2) and SEQ ID NO:60 (CDR3).

In one preferred embodiment, the T-cell engaging protein of the invention comprises a first antigen binding unit specifically binding to DLL3, comprising light chain CDRs comprising the amino acid sequences of SEQ ID NO:1 (CDR1), SEQ ID NO:2 (CDR2) and SEQ ID NO:3 (CDR3) and heavy chain CDRs comprising the amino acid sequences of SEQ ID NO:4 (CDR1), SEQ ID NO:5 (CDR2) and SEQ ID NO:6 (CDR3) and a second antigen binding unit specifically binding to CD3 comprising light chain CDRs comprising the amino acid sequences of SEQ ID NO:61 (CDR1), SEQ ID NO:62 (CDR2) and SEQ ID NO:63 (CDR3) and heavy chain CDRs comprising the amino acid sequences of SEQ ID NO:64 (CDR1), SEQ ID NO:65 (CDR2) and SEQ ID NO:66 (CDR3).

In one preferred embodiment, the T-cell engaging protein of the invention comprises a first antigen binding unit specifically binding to DLL3, comprising light chain CDRs comprising the amino acid sequences of SEQ ID NO:7 (CDR1), SEQ ID NO:8 (CDR2) and SEQ ID NO:9 (CDR3) and heavy chain CDRs comprising the amino acid sequences of SEQ ID NO:10 (CDR1), SEQ ID NO:11 (CDR2) and SEQ ID NO:12 (CDR3) and a second antigen binding unit specifically binding to CD3, comprising light chain CDRs comprising the amino acid sequences of SEQ ID NO:61 (CDR1), SEQ ID NO:62 (CDR2) and SEQ ID NO:63 (CDR3) and heavy chain CDRs comprising the amino acid sequences of SEQ ID NO:64 (CDR1), SEQ ID NO:65 (CDR2) and SEQ ID NO:66 (CDR3).

In one preferred embodiment, the T-cell engaging protein of the invention comprises a first antigen binding unit specifically binding to DLL3, comprising light chain CDRs comprising the amino acid sequences of SEQ ID NO:13 (CDR1), SEQ ID NO:14 (CDR2) and SEQ ID NO:15 (CDR3) and heavy chain CDRs comprising the amino acid sequences of SEQ ID NO:16 (CDR1), SEQ ID NO:17 (CDR2) and SEQ ID NO:18 (CDR3) and a second antigen binding unit specifically binding to CD3, comprising light chain CDRs comprising the amino acid sequences of SEQ ID NO:61 (CDR1), SEQ ID NO:62 (CDR2) and SEQ ID NO:63 (CDR3) and heavy chain CDRs comprising the amino acid sequences of SEQ ID NO:64 (CDR1), SEQ ID NO:65 (CDR2) and SEQ ID NO:66 (CDR3).

In one preferred embodiment, the T-cell engaging protein of the invention comprises (i) a first antigen binding unit specifically binding to DLL3 comprising a light chain variable domain comprising the amino acid sequence of SEQ ID NO:37 and a heavy chain variable domain comprising the amino acid sequence of SEQ ID NO:38 and (ii) a second antigen binding unit specifically binding to CD3 comprising a light chain variable domain comprising the amino acid sequence of SEQ ID NO:67 and a heavy chain variable domain comprising the amino acid sequence of SEQ ID NO:68.

In one preferred embodiment, the T-cell engaging protein of the invention comprises (i) a first antigen binding unit specifically binding to DLL3 comprising a light chain variable domain comprising the amino acid sequence of SEQ ID NO:39 and a heavy chain variable domain comprising the amino acid sequence of SEQ ID NO:40 and (ii) a second antigen binding unit specifically binding to CD3 comprising a light chain variable domain comprising the amino acid sequence of SEQ ID NO:67 and a heavy chain variable domain comprising the amino acid sequence of SEQ ID NO:68.

In one preferred embodiment, the T-cell engaging protein of the invention comprises (i) a first antigen binding unit specifically binding to DLL3 comprising a light chain variable domain comprising the amino acid sequence of SEQ ID NO:41 and heavy chain variable domain comprising the amino acid sequence of SEQ ID NO:42 and (ii) a second antigen binding unit specifically binding to CD3 comprising a light chain variable domain comprising the amino acid sequence of SEQ ID NO:67 and a heavy chain variable domain comprising the amino acid sequence of SEQ ID NO:68.

In one preferred embodiment, the T-cell engaging protein of the invention comprises (i) a first antigen binding unit specifically binding to DLL3 comprising a light chain variable domain comprising the amino acid sequence of SEQ ID NO:37 and heavy chain variable domain comprising the amino acid sequence of SEQ ID NO:38 and (ii) a second antigen binding unit specifically binding to CD3 comprising a light chain variable domain comprising the amino acid sequence of SEQ ID NO:69 and a heavy chain variable domain comprising the amino acid sequence of SEQ ID NO:70.

In one preferred embodiment, the T-cell engaging protein of the invention comprises (i) a first antigen binding unit specifically binding to DLL3 comprising a light chain variable domain comprising the amino acid sequence of SEQ ID NO:39 and heavy chain variable domain comprising the amino acid sequence of SEQ ID NO:40 and (ii) a second antigen binding unit specifically binding to CD3 comprising a light chain variable domain comprising the amino acid sequence of SEQ ID NO:69 and a heavy chain variable domain comprising the amino acid sequence of SEQ ID NO:70.

In one preferred embodiment, the T-cell engaging protein of the invention comprises (i) a first antigen binding unit specifically binding to DLL3 comprising a light chain variable domain comprising the amino acid sequence of SEQ ID NO:41 and heavy chain variable domain comprising the amino acid sequence of SEQ ID NO:42 and a second antigen binding unit specifically binding to CD3 comprising a light chain variable domain comprising the amino acid sequence of SEQ ID NO:69 and a heavy chain variable domain comprising the amino acid sequence of SEQ ID NO:70.

In some embodiments of the T-cell engaging protein of the invention, the first and/or the second antigen binding unit further comprises a CL and a CH1 domain like in a light/heavy Fab domain of a conventional antibody molecule, thus said first binding unit comprises a) a VL domain (e.g., defined by the light chain CDR (LCCDR) or VL sequences of any one of DLL3 #1, DLL3 #2, DLL3 #3) covalently linked (directly or indirectly bound) to a first CL domain and b) a VH domain (e.g., defined by the heavy chain CDR (HCCDR) or VH sequences of any one of DLL3 #1, DLL3 #2, DLL3 #3,) covalently linked (directly or indirectly bound) to a first CH1 domain and/or said second antigen binding unit comprises a) a VL domain (e.g., defined by the LCCDR or VL sequences of CD3 #1 or CD3 #2) covalently linked (directly or indirectly bound) to a second CL domain and b) a VH domain (e.g., defined by HCCDR or VH sequences of CD3 #1 or CD3 #2) covalently linked (directly or indirectly bound) to a second CH1 domain.

For an explanation of the designations comprising a number sign (“#”), please see the table further below. In the context of the present invention, a CL domain is the constant domain of an antibody light chain, for example a kappa (┘) or a lambda (┘) light chain. An example of a constant region of a kappa light chain is shown in SEQ ID NO:87. An example of a constant region of a lambda light chain is shown in SEQ ID NO:88. In some embodiments, the first and the second CL domain are the same, e.g. the first and the second CL domain are both a kappa light chain constant domain or the first and the second CL domain are both a lambda light chain constant domain. In some embodiments, the first and the second CL domain are different, e.g., the first CL domain is a constant kappa domain and the second CL domain is a constant lambda domain or vice versa.

In the context of the present invention, a CH1 domain is the first constant domain of an antibody heavy chain. An example of a constant CH1 domain is shown in SEQ ID NO:90.

In a preferred embodiment, the T-cell engaging protein of the invention comprises a first single chain Fab forming a first antigen binding unit comprising the sequence of SEQ ID NO:49 and a second single chain Fab comprising the sequence of SEQ ID NO:71 forming a second antigen binding unit. In a preferred embodiment, the T-cell engaging protein of the invention comprises a first single chain Fab forming a first antigen binding unit comprising the sequence of SEQ ID NO:50 and a second single chain Fab comprising the sequence of SEQ ID NO:71 forming a second antigen binding unit. In a preferred embodiment, the T-cell engaging protein of the invention comprises a first single chain Fab forming a first antigen binding unit comprising the sequence of SEQ ID NO:51 and a second single chain Fab comprising the sequence of SEQ ID NO:71 forming a second antigen binding unit.

In a preferred embodiment, the T-cell engaging protein of the invention comprises a first single chain Fab forming a first antigen binding unit comprising the sequence of SEQ ID NO:49 and a second single chain Fab comprising the sequence of SEQ ID NO:72 forming a second antigen binding unit. In a preferred embodiment, the T-cell engaging protein of the invention comprises a first single chain Fab forming a first antigen binding unit comprising the sequence of SEQ ID NO:50 and a second single chain Fab comprising the sequence of SEQ ID NO:72 forming a second antigen binding unit. In a preferred embodiment, the T-cell engaging protein of the invention comprises a first single chain Fab forming a first antigen binding unit comprising the sequence of SEQ ID NO:51 and a second single chain Fab comprising the sequence of SEQ ID NO:72 forming a second antigen binding unit.

In preferred embodiments, the T-cell engaging protein of the invention comprises a first polypeptide chain comprising a first single chain Fab specifically binding to DLL3 (e.g., any one of DLL3 #1, DLL3 #2, DLL3 #3 as defined by the respective CDR or VH/VL sequences above) and a first Fc domain (this polypeptide chain herein referred to also as “DLL3 chain”) and a second polypeptide chain comprising a second single chain Fab specifically binding to CD3 (e.g. CD3 #1 or CD3 #2 as defined by the respective CDR or VH/VL sequences above) and a second Fc domain (this polypeptide chain herein referred to herein also as “CD3 chain”). In some embodiments, the first and the second Fc domain are the same. In preferred embodiments, the first and the second Fe domains are different. The resulting T-cell engaging protein of the invention bear a full Fe and have two independent binding sites, a first binding unit for DLL3 and a second binding unit for CD3.

In another preferred embodiment, said bispecific T-cell engaging protein comprises a first polypeptide chain specifically binding to DLL3 and a second polypeptide chain specifically binding to CD3, wherein the first chain comprises a first light chain covalently linked (e.g. directly bound) to a first linker, which is itself covalently linked (e.g. directly bound) to a first heavy chain, and wherein the second chain specifically binding to CD3 comprises a second light chain covalently linked (e.g. directly bound) to a second linker, which is itself covalently linked (e.g. directly bound) to a second heavy chain.

In some embodiments, starting from its N-terminus, the first polypeptide chain comprises a first light chain variable region specifically binding to DLL3, a first light chain constant region, a first linker, a first heavy chain variable region specific for DLL3 and a first heavy chain constant region. In some embodiments, starting from its N-terminus, the second polypeptide chain comprises a second light chain variable region specifically binding to CD3, a second light chain constant region, a second linker, a second heavy chain variable region specific for CD3 and a second heavy chain constant region.

The resulting proteins bear a full Fe, which is marginally larger than an IgG (mostly owing to said first and second linker) and has two independent binding sites (e.g., each binding site being monovalent for the respective antigen), a first binding site for DLL3 and a second binding site for CD3. Preferably, the first and second polypeptide chain are linked via one or more disulfide bonds. As such, preferred bispecific-cell engaging protein of the invention are antibody-like structures, having the Y shaped structure of a conventional full length antibody. This bispecific format greatly reduces heterogeneity after expression and purification (e.g. by avoiding mispairing of light and heavy variable domains with different binding specificities), while maintaining the functional properties of the binding moieties within a structure less likely to generate unwanted immunogenic reactions. This also enables good expression of heterodimeric proteins, e.g. in mammalian cells.

In one preferred embodiment, the T-cell engaging protein comprises a first polypeptide chain specific for DLL3 comprising an amino acid sequence of SEQ ID NO:73 and a second polypeptide chain specific for CD3 comprising the amino acid sequence of SEQ ID NO:79.

In one preferred embodiment, the T-cell engaging protein comprises a first polypeptide chain specific for DLL3 comprising an amino acid sequence of SEQ ID NO:74 and a second polypeptide chain specific for CD3 comprising the amino acid sequence of SEQ ID NO:79.

In one preferred embodiment, the T-cell engaging protein comprises a first polypeptide chain specific for DLL3 comprising an amino acid sequence of SEQ ID NO:75 and a second polypeptide chain specific for CD3 comprising the amino acid sequence of SEQ ID NO:79, in the examples below also referred to as BI 764532.

In one preferred embodiment, the T-cell engaging protein comprises a first polypeptide chain specific for DLL3 comprising an amino acid sequence of SEQ ID NO:73 and a second polypeptide chain specific for CD3 comprising the amino acid sequence of SEQ ID NO:80.

In one preferred embodiment, the T-cell engaging protein comprises a first polypeptide chain specific for DLL3 comprising an amino acid sequence of SEQ ID NO:74 and a second polypeptide chain specific for CD3 comprising the amino acid sequence of SEQ ID NO:80.

In one preferred embodiment, the T-cell engaging protein comprises a first polypeptide chain specific for DLL3 comprising an amino acid sequence of SEQ ID NO:75 and a second polypeptide chain specific for CD3 comprising the amino acid sequence of SEQ ID NO:80.

Amino acid sequences defining preferred T-cell engaging proteins or parts thereof are set forth below. The SEQ ID NOs are those which are used in the sequence listing accompanying this application. In case of discrepancies, the sequences given in the table below or in WO 2019/234220 shall prevail.

TABLE 2

| | Brief | |
|---|---|---|
| | description | |
| SEQ ID | of | |
| Number | sequence | Sequence |
| SEQ ID | DLL3#1 | RASQSVSSNFLV |
| NO: 1 | LCCDR1 | |
| SEQ ID | DLL3#1 | GASTRAS |
| NO: 2 | LCCDR2 | |
| SEQ ID | DLL3#1 | QQYGDSPYT |
| NO: 3 | LCCDR3 | |
| SEQ ID | DLL3#1 | GNTFTNYYMH |
| NO: 4 | HCCDR1 | |
| SEQ ID | DLL3#1 | IIDPSVGSKSYAQKFLG |
| NO: 5 | HCCDR2 | |
| SEQ ID | DLL3#1 | AGKRFGESYFDY |
| NO: 6 | HCCDR3 | |
| SEQ ID | DLL3#2 | RASQGISNYLA |
| NO: 7 | LCCDR1 | |
| SEQ ID | DLL3#2 | AASSLQS |
| NO: 8 | LCCDR2 | |
| SEQ ID | DLL3#2 | LQHNSSPYT |
| NO: 9 | LCCDR3 | |
| SEQ ID | DLL3#2 | GYTFTSYYMH |
| NO: 10 | HCCDR1 | |
| SEQ ID | DLL3#2 | IINPSGGSTSYAQKFQG |
| NO: 11 | HCCDR2 | |
| SEQ ID | DLL3#2 | GEAVGGNYYYYGMDV |
| NO: 12 | HCCDR3 | |
| SEQ ID | DLL3#3 | RASQGISNYLV |
| NO: 13 | LCCDR1 | |
| SEQ ID | DLL3#3 | AVSSLYS |
| NO: 14 | LCCDR2 | |
| SEQ ID | DLL3#3 | LQHDSYPYT |
| NO: 15 | LCCDR3 | |
| SEQ ID | DLL3#3 | GYTFTSYYVH |
| NO: 16 | HCCDR1 | |
| SEQ ID | DLL3#3 | IINPGGGTTSYAQKFLG |
| NO: 17 | HCCDR2 | |
| SEQ ID | DLL3#3 | GEAVTGNYFYYGMDV |
| NO: 18 | HCCDR3 | |
| SEQ ID | DLL3#1 | EIVLTQSPGTLSLSPGERATLSCRASQSVSSNFLVWYQQKPGQAPR |
| NO: 37 | VL | PLIYGASTRASGIPDRFSGSGSGADFTLTISRLEPEDFALYYCQQY |
| | | GDSPYTFGQGTTLEIK |
| SEQ ID | DLL3#1 | QVQLVQSGAEVKKPGASVKVSCKASGNTFTNYYMHWVRQAPGPGLE |
| NO: 38 | VH | WMGIIDPSVGSKSYAQKFLGRVTIARDTSTSTVFLDLYSLRSEDTA |
| | | VYFCARAGKRFGESYFDYWGQGTLVTVSS |
| SEQ ID | DLL3#2 | DIQMTQSPSAMSASVGDRVTITCRASQGISNYLAWFQQKPGKVPEP |
| NO: 39 | VL | LIYAASSLQSGVPSRFSGSGSVTEFTLTISSLQPEDFATYYCLQHN |
| | | SSPYTFGQGTKLEIK |
| SEQ ID | DLL3#2 | QVQLVQSGAEVKKPGASVKVSCKASGYTFTSYYMHWVRQAPGQGLE |
| NO: 40 | VH | WMGIINPSGGSTSYAQKFQGRVTMTRDTSTSTVYMELSSLRSEDTA |
| | | VYYCARGEAVGGNYYYYGMDVWGQGTTVTVSS |
| SEQ ID | DLL3#3 | DIQMTQSPSAMSASVGDRVTITCRASQGISNYLVWFQQKPGKAPKR |
| NO: 41 | VL | LIYAVSSLYSGVPSRFSGSGSGTEFTLTISSLQPEDFATYYCLQHD |
| | | SYPYTFGQGTKLEIK |
| SEQ ID | DLL3#3 | QVQLVQSGAEVKKPGASVKVSCKASGYTFTSYYVHWVRQAPGQGLE |
| NO: 42 | VH | WMVIINPGGGTTSYAQKFLGRVTMTRDTSTNTVYMELKSLRSEDTA |
| | | VYYCARGEAVTGNYFYYGMDVWGQGTTVTVSS |
| SEQ ID | DLL3#1 | EIVLTQSPGTLSLSPGERATLSCRASQSVSSNFLVWYQQKPGQAPR |
| NO: 49 | sc Fab | PLIYGASTRASGIPDRESGSGSGADFTLTISRLEPEDFALYYCQQY |
| | | GDSPYTFGQGTTLEIKRTVAAPSVFIFPPSDEQLKSGTASVVCLLN |
| | | NFYPREAKVQWKVDNALQSGNSQESVTEQDSKDSTYSLSSTLTLSK |
| | | ADYEKHKVYACEVTHQGLSSPVTKSFNRGECGGGGSEGKSSGSGSE |
| | | SKSTEGKSSGSGSESKSTGGGGSQVQLVQSGAEVKKPGASVKVSCK |
| | | ASGNTFTNYYMHWVRQAPGPGLEWMGIIDPSVGSKSYAQKFLGRVT |
| | | IARDTSTSTVFLDLYSLRSEDTAVYFCARAGKRFGESYFDYWGQGT |
| | | LVTVSSASTKGPSVFPLAPSSKSTSGGTAALGCLVKDYFPEPVTVS |
| | | WNSGALTSGVHTFPAVLQSSGLYSLSSVVTVPSSSLGTQTYICNVN |
| | | HKPSNTKVDKRVEPKSC |
| SEQ ID | DLL3#2 | DIQMTQSPSAMSASVGDRVTITCRASQGISNYLAWFQQKPGKVPEP |
| NO: 50 | sc Fab | LIYAASSLQSGVPSRFSGSGSVTEFTLTISSLQPEDFATYYCLQHN |
| | | SSPYTFGQGTKLEIKRTVAAPSVFIFPPSDEQLKSGTASVVCLLNN |
| | | FYPREAKVQWKVDNALQSGNSQESVTEQDSKDSTYSLSSTLTLSKA |
| | | DYEKHKVYACEVTHQGLSSPVTKSFNRGECGGGGSEGKSSGSGSES |
| | | KSTEGKSSGSGSESKSTGGGGSQVQLVQSGAEVKKPGASVKVSCKA |
| | | SGYTFTSYYMHWVRQAPGQGLEWMGIINPSGGSTSYAQKFQGRVTM |
| | | TRDTSTSTVYMELSSLRSEDTAVYYCARGEAVGGNYYYYGMDVWGQ |
| | | GTTVTVSSASTKGPSVFPLAPSSKSTSGGTAALGCLVKDYFPEPVT |
| | | VSWNSGALTSGVHTFPAVLQSSGLYSLSSVVTVPSSSLGTQTYICN |
| | | VNHKPSNTKVDKRVEPKSC |
| SEQ ID | DLL3#3 | DIQMTQSPSAMSASVGDRVTITCRASQGISNYLVWFQQKPGKAPKR |
| NO: 51 | scFab | LIYAVSSLYSGVPSRFSGSGSGTEFTLTISSLQPEDFATYYCLQHD |
| | | SYPYTFGQGTKLEIKRTVAAPSVFIFPPSDEQLKSGTASVVCLLNN |
| | | FYPREAKVQWKVDNALQSGNSQESVTEQDSKDSTYSLSSTLTLSKA |
| | | DYEKHKVYACEVTHQGLSSPVTKSFNRGECGGGGSEGKSSGSGSES |
| | | KSTEGKSSGSGSESKSTGGGGSQVQLVQSGAEVKKPGASVKVSCKA |
| | | SGYTFTSYYVHWVRQAPGQGLEWMVIINPGGGTTSYAQKFLGRVTM |
| | | TRDTSTNTVYMELKSLRSEDTAVYYCARGEAVTGNYFYYGMDVWGQ |
| | | GTTVTVSSASTKGPSVFPLAPSSKSTSGGTAALGCLVKDYFPEPVT |
| | | VSWNSGALTSGVHTFPAVLQSSGLYSLSSVVTVPSSSLGTQTYICN |
| | | VNHKPSNTKVDKRVEPKSC |
| SEQ ID | CD3#1 | RSSTGAVTTSNYAN |
| NO: 55 | LCCDR1 | |
| SEQ ID | CD3#1 | GTNKRAP |
| NO: 56 | LCCDR2 | |
| SEQ ID | CD3#1 | ALWYSNLWV |
| NO: 57 | LCCDR3 | |
| SEQ ID | CD3#1 | GFTFNTYAMN |
| NO: 58 | HCCDR1 | |
| SEQ ID | CD3#1 | RIRSKYNNYATYYADSVKD |
| NO: 59 | HCCDR2 | |
| SEQ ID | CD3#1 | HGNFGNSYVSWFAY |
| NO: 60 | HCCDR3 | |
| SEQ ID | CD3#2 | RSSTGAVTTSNYAN |
| NO: 61 | LCCDR1 | |
| SEQ ID | CD3#2 | GTNKRAP |
| NO: 62 | LCCDR2 | |
| SEQ ID | CD3#2 | ALWYSNLWV |
| NO: 63 | LCCDR3 | |
| SEQ ID | CD3#2 | GFTFNTYAMN |
| NO: 64 | HCCDR1 | |
| SEQ ID | CD3#2 | RIRSKYINYATYYADSVKD |
| NO: 65 | HCCDR2 | |
| SEQ ID | CD3#2 | HGNFGNSYVSWFAY |
| NO: 66 | HCCDR3 | |
| SEQ ID | CD3#1 | EAVVTQEPSLTVSPGGTVTLTCRSSTGAVTTSNYANWVQEKPGQLP |
| NO: 67 | VL | RGLIGGTNKRAPWVPARFSGSLLGGKAALTLSGAQPEDEAEYFCAL |
| | | WYSNLWVFGGGTKLTVL |
| SEQ ID | CD3#1 | EVQLVESGGGLVQPGGSLKLSCAASGFTFNTYAMNWVRQAPGKGLE |
| NO: 68 | VH | WVARIRSKYNNYATYYADSVKDRFTISRDDSKNTAYLQMNNLKTED |
| | | TAVYYCVRHGNFGNSYVSWFAYWGQGTLVTVSA |
| SEQ ID | CD3#2 | EAVVTQEPSLTVSPGGTVTLTCRSSTGAVTTSNYANWVQEKPGQLP |
| NO: 69 | VL | RGLIGGTNKRAPWVPARFSGSLLGGKAALTLSGAQPEDEAEYFCAL |
| | | WYSNLWVFGGGTKLTVL |
| SEQ ID | CD3#2 | EVQLVESGGGLVQPGGSLKLSCAASGFTFNTYAMNWVRQAPGKGLE |
| NO: 70 | VH | WVARIRSKYINYATYYADSVKDRFTISRDDSKNTAYLQMNNLKTED |
| | | TAVYYCVRHGNFGNSYVSWFAYWGQGTLVTVSA |
| SEQ ID | CD3#1 | EAVVTQEPSLTVSPGGTVTLTCRSSTGAVTTSNYANWVQEKPGQLP |
| NO: 71 | scFab | RGLIGGTNKRAPWVPARFSGSLLGGKAALTLSGAQPEDEAEYFCAL |
| | | WYSNLWVFGGGTKLTVLGQPKAAPSVTLFPPSSEELQANKATLVCL |
| | | ISDFYPGAVKVAWKADGSPVNTGVETTTPSKQSNNKYAASSYLSLT |
| | | PEQWKSHRSYSCQVTHEGSTVEKTVAPAECSGGGGSEGKSSGSGSE |
| | | SKSTEGKSSGSGSESKSTGGGGSEVQLVESGGGLVQPGGSLKLSCA |
| | | ASGFTFNTYAMNWVRQAPGKGLEWVARIRSKYNNYATYYADSVKDR |
| | | FTISRDDSKNTAYLQMNNLKTEDTAVYYCVRHGNFGNSYVSWFAYW |
| | | GQGTLVTVSAASTKGPSVFPLAPSSKSTSGGTAALGCLVKDYFPEP |
| | | VTVSWNSGALTSGVHTFPAVLQSSGLYSLSSVVTVPSSSLGTQTYI |
| | | CNVNHKPSNTKVDKRVEPKSC |
| SEQ ID | CD3#2 | EAVVTQEPSLTVSPGGTVTLTCRSSTGAVTTSNYANWVQEKPGQLP |
| NO: 72 | sc Fab | RGLIGGTNKRAPWVPARFSGSLLGGKAALTLSGAQPEDEAEYFCAL |
| | | WYSNLWVFGGGTKLTVLGQPKAAPSVTLFPPSSEELQANKATLVCL |
| | | ISDFYPGAVKVAWKADGSPVNTGVETTTPSKQSNNKYAASSYLSLT |
| | | PEQWKSHRSYSCQVTHEGSTVEKTVAPAECSGGGGSEGKSSGSGSE |
| | | SKSTEGKSSGSGSESKSTGGGGSEVQLVESGGGLVQPGGSLKLSCA |
| | | ASGFTFNTYAMNWVRQAPGKGLEWVARIRSKYINYATYYADSVKDR |
| | | FTISRDDSKNTAYLQMNNLKTEDTAVYYCVRHGNFGNSYVSWFAYW |
| | | GQGTLVTVSAASTKGPSVFPLAPSSKSTSGGTAALGCLVKDYFPEP |
| | | VTVSWNSGALTSGVHTFPAVLQSSGLYSLSSVVTVPSSSLGTQTYI |
| | | CNVNHKPSNTKVDKRVEPKSC |
| SEQ ID | DLL3#1 | EIVLTQSPGTLSLSPGERATLSCRASQSVSSNFLVWYQQKPGQAPR |
| NO: 73 | chain | PLIYGASTRASGIPDRESGSGSGADFTLTISRLEPEDFALYYCQQY |
| | | GDSPYTFGQGTTLEIKRTVAAPSVFIFPPSDEQLKSGTASVVCLLN |
| | | NFYPREAKVQWKVDNALQSGNSQESVTEQDSKDSTYSLSSTLTLSK |
| | | ADYEKHKVYACEVTHQGLSSPVTKSFNRGECGGGGSEGKSSGSGSE |
| | | SKSTEGKSSGSGSESKSTGGGGSQVQLVQSGAEVKKPGASVKVSCK |
| | | ASGNTFTNYYMHWVRQAPGPGLEWMGIIDPSVGSKSYAQKFLGRVT |
| | | IARDTSTSTVFLDLYSLRSEDTAVYFCARAGKRFGESYFDYWGQGT |
| | | LVTVSSASTKGPSVFPLAPSSKSTSGGTAALGCLVKDYFPEPVTVS |
| | | WNSGALTSGVHTFPAVLQSSGLYSLSSVVTVPSSSLGTQTYICNVN |
| | | HKPSNTKVDKRVEPKSCDKTHTCPPCPAPEAAGGPSVFLFPPKPKD |
| | | TLMISRTPEVTCVVVDVSHEDPEVKFNWYVDGVEVHNAKTKPREEQ |
| | | YNSTYRVVSVLTVLHQDWLNGKEYKCKVSNKALPAPIEKTISKAKG |
| | | QPREPQVYTLPPSREEMTKNQVSLWCLVKGFYPSDIAVEWESNGQP |
| | | ENNYKTTPPVLDSDGSFFLYSKLTVDKSRWQQGNVFSCSVMHEALH |
| | | NHYTQKSLSLSPG |
| SEQ ID | DLL3#2 | DIQMTQSPSAMSASVGDRVTITCRASQGISNYLAWFQQKPGKVPEP |
| NO: 74 | chain | LIYAASSLQSGVPSRFSGSGSVTEFTLTISSLQPEDFATYYCLQHN |
| | | SSPYTFGQGTKLEIKRTVAAPSVFIFPPSDEQLKSGTASVVCLLNN |
| | | FYPREAKVQWKVDNALQSGNSQESVTEQDSKDSTYSLSSTLTLSKA |
| | | DYEKHKVYACEVTHQGLSSPVTKSFNRGECGGGGSEGKSSGSGSES |
| | | KSTEGKSSGSGSESKSTGGGGSQVQLVQSGAEVKKPGASVKVSCKA |
| | | SGYTFTSYYMHWVRQAPGQGLEWMGIINPSGGSTSYAQKFQGRVTM |
| | | TRDTSTSTVYMELSSLRSEDTAVYYCARGEAVGGNYYYYGMDVWGQ |
| | | GTTVTVSSASTKGPSVFPLAPSSKSTSGGTAALGCLVKDYFPEPVT |
| | | VSWNSGALTSGVHTFPAVLQSSGLYSLSSVVTVPSSSLGTQTYICN |
| | | VNHKPSNTKVDKRVEPKSCDKTHTCPPCPAPEAAGGPSVFLFPPKP |
| | | KDTLMISRTPEVTCVVVDVSHEDPEVKFNWYVDGVEVHNAKTKPRE |
| | | EQYNSTYRVVSVLTVLHQDWLNGKEYKCKVSNKALPAPIEKTISKA |
| | | KGQPREPQVYTLPPSREEMTKNQVSLWCLVKGFYPSDIAVEWESNG |
| | | QPENNYKTTPPVLDSDGSFFLYSKLTVDKSRWQQGNVFSCSVMHEA |
| | | LHNHYTQKSLSLSPG |
| SEQ ID | DLL3#3 | DIQMTQSPSAMSASVGDRVTITCRASQGISNYLVWFQQKPGKAPKR |
| NO: 75 | chain | LIYAVSSLYSGVPSRFSGSGSGTEFTLTISSLQPEDFATYYCLQHD |
| | | SYPYTFGQGTKLEIKRTVAAPSVFIFPPSDEQLKSGTASVVCLLNN |
| | | FYPREAKVQWKVDNALQSGNSQESVTEQDSKDSTYSLSSTLTLSKA |
| | | DYEKHKVYACEVTHQGLSSPVTKSFNRGECGGGGSEGKSSGSGSES |
| | | KSTEGKSSGSGSESKSTGGGGSQVQLVQSGAEVKKPGASVKVSCKA |
| | | SGYTFTSYYVHWVRQAPGQGLEWMVIINPGGGTTSYAQKFLGRVTM |
| | | TRDTSTNTVYMELKSLRSEDTAVYYCARGEAVTGNYFYYGMDVWGQ |
| | | GTTVTVSSASTKGPSVFPLAPSSKSTSGGTAALGCLVKDYFPEPVT |
| | | VSWNSGALTSGVHTFPAVLQSSGLYSLSSVVTVPSSSLGTQTYICN |
| | | VNHKPSNTKVDKRVEPKSCDKTHTCPPCPAPEAAGGPSVFLFPPKP |
| | | KDTLMISRTPEVTCVVVDVSHEDPEVKFNWYVDGVEVHNAKTKPRE |
| | | EQYNSTYRVVSVLTVLHQDWLNGKEYKCKVSNKALPAPIEKTISKA |
| | | KGQPREPQVYTLPPSREEMTKNQVSLWCLVKGFYPSDIAVEWESNG |
| | | QPENNYKTTPPVLDSDGSFFLYSKLTVDKSRWQQGNVFSCSVMHEA |
| | | LHNHYTQKSLSLSPG |
| SEQ ID | CD3#1 | EAVVTQEPSLTVSPGGTVTLTCRSSTGAVTTSNYANWVQEKPGQLP |
| NO: 79 | chain | RGLIGGTNKRAPWVPARFSGSLLGGKAALTLSGAQPEDEAEYFCAL |
| | | WYSNLWVFGGGTKLTVLGQPKAAPSVTLFPPSSEELQANKATLVCL |
| | | ISDFYPGAVKVAWKADGSPVNTGVETTTPSKQSNNKYAASSYLSLT |
| | | PEQWKSHRSYSCQVTHEGSTVEKTVAPAECSGGGGSEGKSSGSGSE |
| | | SKSTEGKSSGSGSESKSTGGGGSEVQLVESGGGLVQPGGSLKLSCA |
| | | ASGFTFNTYAMNWVRQAPGKGLEWVARIRSKYNNYATYYADSVKDR |
| | | FTISRDDSKNTAYLQMNNLKTEDTAVYYCVRHGNFGNSYVSWFAYW |
| | | GQGTLVTVSAASTKGPSVFPLAPSSKSTSGGTAALGCLVKDYFPEP |
| | | VTVSWNSGALTSGVHTFPAVLQSSGLYSLSSVVTVPSSSLGTQTYI |
| | | CNVNHKPSNTKVDKRVEPKSCDKTHTCPPCPAPEAAGGPSVFLFPP |
| | | KPKDTLMISRTPEVTCVVVDVSHEDPEVKFNWYVDGVEVHNAKTKP |
| | | REEQYNSTYRVVSVLTVLHQDWLNGKEYKCKVSNKALPAPIEKTIS |
| | | KAKGQPREPQVYTLPPSREEMTKNQVSLSCAVKGFYPSDIAVEWES |
| | | NGQPENNYKTTPPVLDSDGSFFLVSKLTVDKSRWQQGNVFSCSVMH |
| | | EALHNRFTQKSLSLSPG |
| SEQ ID | CD3#2 | EAVVTQEPSLTVSPGGTVTLTCRSSTGAVTTSNYANWVQEKPGQLP |
| NO: 80 | chain | RGLIGGTNKRAPWVPARFSGSLLGGKAALTLSGAQPEDEAEYFCAL |
| | | WYSNLWVFGGGTKLTVLGQPKAAPSVTLFPPSSEELQANKATLVCL |
| | | ISDFYPGAVKVAWKADGSPVNTGVETTTPSKQSNNKYAASSYLSLT |
| | | PEQWKSHRSYSCQVTHEGSTVEKTVAPAECSGGGGSEGKSSGSGSE |
| | | SKSTEGKSSGSGSESKSTGGGGSEVQLVESGGGLVQPGGSLKLSCA |
| | | ASGFTFNTYAMNWVRQAPGKGLEWVARIRSKYINYATYYADSVKDR |
| | | FTISRDDSKNTAYLQMNNLKTEDTAVYYCVRHGNFGNSYVSWFAYW |
| | | GQGTLVTVSAASTKGPSVFPLAPSSKSTSGGTAALGCLVKDYFPEP |
| | | VTVSWNSGALTSGVHTFPAVLQSSGLYSLSSVVTVPSSSLGTQTYI |
| | | CNVNHKPSNTKVDKRVEPKSCDKTHTCPPCPAPEAAGGPSVFLFPP |
| | | KPKDTLMISRTPEVTCVVVDVSHEDPEVKFNWYVDGVEVHNAKTKP |
| | | REEQYNSTYRVVSVLTVLHQDWLNGKEYKCKVSNKALPAPIEKTIS |
| | | KAKGQPREPQVYTLPPSREEMTKNQVSLSCAVKGFYPSDIAVEWES |
| | | NGQPENNYKTTPPVLDSDGSFFLVSKLTVDKSRWQQGNVFSCSVMH |
| | | EALHNRFTQKSLSLSPG |
| SEQ ID | Fc | DKTHTCPPCPAPELLGGPSVFLFPPKPKDTLMISRTPEVTCVVVDV |
| NO: 81 | domain* | SHEDPEVKFNWYVDGVEVHNAKTKPREEQYNSTYRVVSVLTVLHQD |
| | (IgG1) | WLNGKEYKCKVSNKALPAPIEKTISKAKGQPREPQVYTLPPSREEM |
| | | TKNQVSLTCLVKGFYPSDIAVEWESNGQPENNYKTTPPVLDSDGSF |
| | | FLYSKLTVDKSRWQQGNVFSCSVMHEALHNHYTQKSLSLSPG |
| SEQ ID | Fc W | DKTHTCPPCPAPEAAGGPSVFLFPPKPKDTLMISRTPEVTCVVVDV |
| NO: 82 | domain | SHEDPEVKFNWYVDGVEVHNAKTKPREEQYNSTYRVVSVLTVLHQD |
| | (IgG1, | WLNGKEYKCKVSNKALPAPIEKTISKAKGQPREPQVYTLPPSREEM |
| | LALA) | TKNQVSLWCLVKGFYPSDIAVEWESNGQPENNYKTTPPVLDSDGSF |
| | | FLYSKLTVDKSRWQQGNVFSCSVMHEALHNHYTQKSLSLSPG |
| SEQ ID | Fc SAV | DKTHTCPPCPAPEAAGGPSVFLFPPKPKDTLMISRTPEVTCVVVDV |
| NO: 83 | domain | SHEDPEVKFNWYVDGVEVHNAKTKPREEQYNSTYRVVSVLTVLHQD |
| | (IgG1, | WLNGKEYKCKVSNKALPAPIEKTISKAKGQPREPQVYTLPPSREEM |
| | RF/LALA) | TKNQVSLSCAVKGFYPSDIAVEWESNGQPENNYKTTPPVLDSDGSF |
| | | FLVSKLTVDKSRWQQGNVFSCSVMHEALHNRFTQKSLSLSPG |
| SEQ ID | Fc domain | ESKYGPPCPPCPAPEFLGGPSVFLFPPKPKDTLMISRTPEVTCVVV |
| NO: 84 | (IgG4Pro) | DVSQEDPEVQFNWYVDGVEVHNAKTKPREEQFNSTYRVVSVLTVLH |
| | | QDWLNGKEYKCKVSNKGLPSSIEKTISKAKGQPREPQVYTLPPSQE |
| | | EMTKNQVSLTCLVKGFYPSDIAVEWESNGQPENNYKTTPPVLDSDG |
| | | SFFLYSRLTVDKSRWQEGNVFSCSVMHEALHNHYTQKSLSLSLG |
| SEQ ID | Fc W | ESKYGPPCPPCPAPEFLGGPSVFLFPPKPKDTLMISRTPEVTCVVV |
| NO: 85 | domain | DVSQEDPEVQFNWYVDGVEVHNAKTKPREEQFNSTYRVVSVLTVLH |
| | (IgG4Pro) | QDWLNGKEYKCKVSNKGLPSSIEKTISKAKGQPREPQVYTLPPSQE |
| | | EMTKNQVSLWCLVKGFYPSDIAVEWESNGQPENNYKTTPPVLDSDG |
| | | SFFLYSRLTVDKSRWQEGNVFSCSVMHEALHNHYTQKSLSLSLG |
| SEQ ID | Fc SAV | ESKYGPPCPPCPAPEFLGGPSVFLFPPKPKDTLMISRTPEVTCVVV |
| NO: 86 | domain | DVSQEDPEVQFNWYVDGVEVHNAKTKPREEQFNSTYRVVSVLTVLH |
| | (IgG4Pro, | QDWLNGKEYKCKVSNKGLPSSIEKTISKAKGQPREPQVYTLPPSQE |
| | RF) | EMTKNQVSLSCAVKGFYPSDIAVEWESNGQPENNYKTTPPVLDSDG |
| | | SFFLVSRLTVDKSRWQEGNVFSCSVMHEALHNRFTQKSLSLSLG |
| SEQ ID | constant | RTVAAPSVFIFPPSDEQLKSGTASVVCLLNNFYPREAKVQWKVDNA |
| NO: 87 | region of a | LQSGNSQESVTEQDSKDSTYSLSSTLTLSKADYEKHKVYACEVTHQ |
| | kappa light | GLSSPVTKSFNRGEC |
| | chain | |
| SEQ ID | constant | GQPKAAPSVTLFPPSSEELQANKATLVCLISDFYPGAVKVAWKADG |
| NO: 88 | region of a | SPVNTGVETTTPSKQSNNKYAASSYLSLTPEQWKSHRSYSCQVTHE |
| | lambda | GSTVEKTVAPAECS |
| | light chain | |
| SEQ ID | Linker | GGGGSEGKSSGSGSESKSTEGKSSGSGSESKSTGGGGS |
| NO: 89 | | |
| SEQ ID | Constant | ASTKGPSVFPLAPSSKSTSGGTAALGCLVKDYFPEPVTVSWNSGAL |
| NO: 90 | region of | TSGVHTFPAVLQSSGLYSLSSVVTVPSSSLGTQTYICNVNHKPSNT |
| | heavy | KVDKRVEPKSC |
| | chain | |
| | CH1 | |

### SPECIFICALLY PREFERRED EMBODIMENTS

In some embodiments, the present invention is related to a T-cell engaging protein comprising a first antigen binding unit that specifically binds to DLL3 and a second antigen binding unit that specifically binds to CD3 for use in a method of treatment of a DLL3-positive cancer or a cancer selected from a Neuroendocrine Neoplasm (NEN), Merkel cell carcinoma (MCC), medullary thyroidal carcinoma and glioma,
the method comprising a step-in cycle, said step-in cycle comprising the steps of:
   administering to a subject an initial dose of said T-cell engaging protein, said initial dose ranging from about 5 μg/kg of the subject's body weight to about 40 μg/kg of the subject's body weight,
   administering to the subject an elevated dose, which is elevated as compared to said initial dose, of said T-cell engaging protein, the elevated dose ranging from about 15 μg/kg of the subject's body weight to about 100 μg/kg of the subject's body weight, or the elevated dose ranging from about 5 mg to about 20 mg, and
   administering to the subject a further dose of the T-cell engaging protein, said further dose ranging from about 80 μg/kg of the subject's body weight to about 1600 μg/kg of the subject's bodyweight, wherein said further dose is equal to or higher than said elevated dose, or said further dose ranging from about 5 mg to about 70 mg, wherein
the T-cell engaging protein is administered by intravenous (IV) infusion,
the subject is a human,
the T-cell engaging protein comprises a first polypeptide chain specific for DLL3 comprising an amino acid sequence of SEQ ID NO:75 and a second polypeptide chain specific for CD3 comprising the amino acid sequence of SEQ ID NO:79, and
wherein said initial, elevated and further doses are administered within three weeks, said initial dose being administered once, wherein said initial dose is administered on day 1, said elevated dose is administered on day 8 and said further dose is administered on day 15 of said step-in cycle.

In some embodiments, the present invention is related to a T-cell engaging protein comprising a first antigen binding unit that specifically binds to DLL3 and a second antigen binding unit that specifically binds to CD3 for use in a method of treatment of a DLL3-positive cancer or a cancer selected from a Neuroendocrine Neoplasm (NEN), Merkel cell carcinoma (MCC), medullary thyroidal carcinoma and glioma,
the method comprising a step-in cycle, said step-in cycle comprising the steps of:
   administering to a subject an initial dose of said T-cell engaging protein, said initial dose ranging from about 5 μg/kg of the subject's body weight to about 15 μg/kg of the subject's body weight,
   administering to the subject an elevated dose, which is elevated as compared to said initial dose, of said T-cell engaging protein, the elevated dose ranging from about 15 μg/kg of the subject's body weight to about 100 μg/kg of the subject's body weight, or the elevated dose ranging from about 5 mg to about 20 mg, and
   administering to the subject a further dose of the T-cell engaging protein, said further dose ranging from about 5 mg to about 70 mg, wherein
the T-cell engaging protein is administered by intravenous (IV) infusion,
the subject is a human,
the T-cell engaging protein comprises a first polypeptide chain specific for DLL3 comprising an amino acid sequence of SEQ ID NO:75 and a second polypeptide chain specific for CD3 comprising the amino acid sequence of SEQ ID NO:79, and
wherein said initial, elevated and further doses are administered within three weeks, said initial dose being administered once, wherein said initial dose is administered on day 1, said elevated dose is administered on day 8 and said further dose is administered on day 15 of said step-in cycle.

In some embodiments, the present invention is related to a T-cell engaging protein comprising a first antigen binding unit that specifically binds to DLL3 and a second antigen binding unit that specifically binds to CD3 for use in a method of treatment of a DLL3-positive cancer or a cancer selected from a Neuroendocrine Neoplasm (NEN), Merkel cell carcinoma (MCC), medullary thyroidal carcinoma and glioma,
the method comprising a step-in cycle, said step-in cycle comprising the steps of:
   administering to a subject an initial dose of said T-cell engaging protein, said initial dose ranging from about 20 μg/kg of the subject's body weight to about 40 μg/kg of the subject's body weight,
   administering to the subject an elevated dose, which is elevated as compared to said initial dose, of said T-cell engaging protein, the elevated dose ranging from about 60 μg/kg of the subject's body weight to about 100 μg/kg of the subject's body weight,
   administering to the subject a further dose of the T-cell engaging protein, said further dose ranging from about 80 μg/kg of the subject's body weight to about 1600 μg/kg of the subject's bodyweight, wherein said further dose is equal to or higher than said elevated dose, wherein
the T-cell engaging protein is administered by intravenous (IV) infusion,
the subject is a human,
the T-cell engaging protein comprises a first polypeptide chain specific for DLL3 comprising an amino acid sequence of SEQ ID NO:75 and a second polypeptide chain specific for CD3 comprising the amino acid sequence of SEQ ID NO:79, and wherein said initial, elevated and further doses are administered within three weeks, said initial dose being administered once, wherein said initial dose is administered on day 1, said elevated dose is administered on day 8 and said further dose is administered on day 15 of said step-in cycle.

In some embodiments, the present invention relates to a T-cell engaging protein comprising a first antigen binding unit that specifically binds to DLL3 and a second antigen binding unit that specifically binds to CD3 for use in a method of treatment of a DLL3-positive cancer or a cancer selected from a Neuroendocrine Neoplasm (NEN), Merkel cell carcinoma (MCC), medullary thyroidal carcinoma and glioma, the method comprising a step-in cycle, the step-in cycle comprising the steps of:
administering to the subject an initial dose, an elevated dose, which is elevated as compared to said initial dose, and a further dose of said T-cell engaging protein, wherein a ratio between said initial, elevated and further dose per kilogram of the subject's body weight is about 1: about 3: about 9, wherein the T-cell engaging protein is administered by intravenous (IV) infusion,
   the subject is a human,
   the T-cell engaging protein comprises a first polypeptide chain specific for DLL3 comprising an amino acid sequence of SEQ ID NO:75 and a second polypeptide chain specific for CD3 comprising the amino acid sequence of SEQ ID NO:79, and
   wherein said initial, elevated and further doses are administered within three weeks, said initial dose being administered once, wherein said initial dose is administered on day 1, said elevated dose is administered on day 8 and said further dose is administered on day 15 of said step-in cycle.

In certain embodiments, the present invention is related to a T-cell engaging protein comprising a first antigen binding unit that specifically binds to DLL3 and a second antigen binding unit that specifically binds to CD3 for use in a method of treatment of a DLL3-positive cancer or a cancer selected from a Neuroendocrine Neoplasm (NEN), Merkel cell carcinoma (MCC), medullary thyroidal carcinoma and glioma,
the method comprising a step-in cycle, the step-in cycle comprising the steps of:
   administering to a subject an initial dose of said T-cell engaging protein, said initial dose ranging from about 5 μg/kg of the subject's body weight to about 15 μg/kg of the subject's body weight,
   administering to the subject an elevated dose, which is elevated as compared to said initial dose, of said T-cell engaging protein, the elevated dose ranging from about 15 μg/kg of the subject's body weight to about 40 μg/kg of the subject's body weight,
   administering to the subject a further dose of said T-cell engaging protein, said further dose ranging from about 80 μg/kg of the subject's body weight to about 1600 μg/kg of the subject's bodyweight, wherein
the T-cell engaging protein is administered by intravenous (IV) infusion,
the subject is a human,
the T-cell engaging protein comprises a first polypeptide chain specific for DLL3 comprising an amino acid sequence of SEQ ID NO:75 and a second polypeptide chain specific for CD3 comprising the amino acid sequence of SEQ ID NO:79, and
wherein said initial, elevated and further doses are administered within three weeks, said initial dose being administered once, wherein said initial dose is administered on day 1, said elevated dose is administered on day 8 and said further dose is administered on day 15 of said step-in cycle.

### The Present Invention Furthermore Relates to the Following Items:

1. A T-cell engaging protein comprising a first antigen binding unit that specifically binds to DLL3 and a second antigen binding unit that specifically binds to CD3 for use in a method of treatment of a DLL3-positive cancer or a cancer selected from a Neuroendocrine Neoplasm (NEN), Merkel cell carcinoma (MCC), medullary thyroidal carcinoma and glioma,
the method comprising a step-in cycle, said step-in cycle comprising the steps of:
   administering to a subject an initial dose of said T-cell engaging protein, said initial dose ranging from about 5 μg/kg of the subject's body weight to about 40 μg/kg of the subject's body weight,
   administering to the subject an elevated dose, which is elevated as compared to said initial dose, of said T-cell engaging protein, the elevated dose ranging from about 15 μg/kg of the subject's body weight to about 100 μg/kg of the subject's body weight, or the elevated dose ranging from about 5 mg to about 20 mg, and
   administering to the subject a further dose of the T-cell engaging protein, said further dose ranging from about 80 μg/kg of the subject's body weight to about 1600 μg/kg of the subject's bodyweight, wherein said further dose is equal to or higher than said elevated dose or said further dose ranging from about 5 mg to about 70 mg.

2. A T-cell engaging protein comprising a first antigen binding unit that specifically binds to DLL3 and a second antigen binding unit that specifically binds to CD3 for use in a method of treatment of a DLL3-positive cancer or a cancer selected from a Neuroendocrine Neoplasm (NEN), Merkel cell carcinoma (MCC), medullary thyroidal carcinoma and glioma,
the method comprising a step-in cycle, said step-in cycle comprising the steps of:
   administering to a subject an initial dose of said T-cell engaging protein, said initial dose ranging from about 5 μg/kg of the subject's body weight to about 15 μg/kg of the subject's body weight,
   administering to the subject an elevated dose, which is elevated as compared to said initial dose, of said T-cell engaging protein, the elevated dose ranging from about 15 μg/kg of the subject's body weight to about 100 μg/kg of the subject's body weight, or the elevated dose ranging from about 5 mg to about 20 mg, and
   administering to the subject a further dose of the T-cell engaging protein, said further dose ranging from about 5 mg to about 70 mg, preferably from about 5 mg to about 10 mg or from about 50 mg to about 70 mg.

3. A T-cell engaging protein comprising a first antigen binding unit that specifically binds to DLL3 and a second antigen binding unit that specifically binds to CD3 for use in a method of treatment of a DLL3-positive cancer or a cancer selected from a Neuroendocrine Neoplasm (NEN), Merkel cell carcinoma (MCC), medullary thyroidal carcinoma and glioma,
the method comprising a step-in cycle, said step-in cycle comprising the steps of:
   administering to a subject an initial dose of said T-cell engaging protein, said initial dose ranging from about 20 μg/kg of the subject's body weight to about 40 μg/kg of the subject's body weight,
   administering to the subject an elevated dose, which is elevated as compared to said initial dose, of said T-cell engaging protein, the elevated dose ranging from about 60 μg/kg of the subject's body weight to about 100 μg/kg of the subject's body weight,
   administering to the subject a further dose of the T-cell engaging protein, said further dose ranging from about 80 μg/kg of the subject's body weight to about 1600 μg/kg of the subject's bodyweight, wherein said further dose is equal to or higher than said elevated dose.

4. The T-cell engaging protein for use according to any of items 1-3, wherein said further dose is equal to a target dose, said target dose being the dose to be administered during a first treatment cycle which optionally directly follows said step-in cycle.

5. The T-cell engaging protein for use according to any one of the preceding items, wherein
said initial dose, said elevated dose and/or said further is/are administered once; or
said initial dose, said elevated dose and/or said further is/are administered more than once, optionally 2 to 10 times,
wherein optionally the number of administrations is determined based on treatment tolerability including presence and/or degree of a cytokine release syndrome (CRS) and/or of an immune effector cell-associated neurotoxicity syndrome (ICANS),
and wherein further optionally said elevated dose follows the initial dose only if said CRS, to the extent determined, is rated grade 1 or less in terms of NCI CTC grade and/or if said ICANS, to the extend determined, is rated grade 1 or less, and/or wherein further optionally
further optionally said further dose follows the elevated dose only if said CRS, to the extent determined, is rated grade 1 or less in terms of NCI CTC grade and/or if said ICANS, to the extend determined, is rated grade 1 or less.

6. The T-cell engaging protein for use according to any one of the preceding items, wherein said initial dose is administered three times to ten times within the step-in cycle.

7. The T-cell engaging protein for use according to any one of the preceding items, wherein said initial dose is administered three times within three weeks, optionally on day 1, day 8 and day 15 of the three weeks.

8. The T-cell engaging protein for use according to any one of the preceding items, wherein said step-in cycle has a duration of at least three weeks.

9. The T-cell engaging protein for use according to any one of the preceding items, wherein said initial, elevated and further doses are administered within three weeks, said initial dose being administered once, wherein optionally said initial dose is administered on day 1, said elevated dose is administered on day 8 and said further dose is administered on day 15 of said step-in cycle.

10. The T-cell engaging protein for use according to any one of the preceding items, wherein said initial dose ranges from about 25 μg/kg of the subject's body weight to about 35 μg/kg of the subject's body weight, and optionally said initial dose is about 30 μg/kg of the subject's body weight;
said elevated dose ranges from about 85 μg/kg of the subject's body weight to about 95 μg/kg of the subject's body weight, and optionally said elevated dose is about 90 μg/kg of the subject's body weight; and
said further dose ranges from about 85 μg/kg of the subject's body weight to about 1550 μg/kg of the subject's body weight, and optionally the further dose is about 90 μg/kg, about 180 μg/kg, about 270 μg/kg, about 360 μg/kg, about 540 μg/kg, about 720 μg/kg, about 1080 μg/kg or about 1530 μg/kg of the subject's body weight.

11. The T-cell engaging protein for use according to any one of the preceding items; the method further comprising a first treatment cycle following the step-in cycle, said first treatment cycle comprising the steps of:
administering to a subject a first target dose of said T-cell engaging protein, said first target dose ranging from about 80 μg/kg of the subject's body weight to about 1600 μg/kg of the subject's body weight,
administering to the subject a second target dose of said T-cell engaging protein, said second dose ranging from about 80 μg/kg of the subject's body weight to about 1600 μg/kg of the subject's body weight, and
administering to the subject a third target dose of said T-cell engaging protein, said third dose ranging from about 80 μg/kg of the subject's body weight to about 1600 μg/kg of the subject's bodyweight,
   wherein optionally the amount of all three target doses is identical.

12. The T-cell engaging protein according to item 11, wherein the amount of all three target doses is identical and the amount is about 90 μg/kg for the first, second and third target dose, respectively.

13. The T-cell engaging protein according to item 11, wherein the amount of all target doses is identical, and the amount is about 180 μg/kg for the first, second and third target dose, respectively.

14. The T-cell engaging protein according to item 11, wherein the amount of all target doses is identical, and the amount is about 270 μg/kg for the first, second and third target dose, respectively.

15. The T-cell engaging protein according to item 11, wherein the amount of all target doses is identical, and the amount is about 360 μg/kg for the first, second and third target dose, respectively.

16. The T-cell engaging protein according to item 11, wherein the amount of all target doses is identical, and the amount is about 540 μg/kg for the first, second and third target dose, respectively.

17. The T-cell engaging protein according to item 11, wherein the amount of all target doses is identical, and the amount is about 720 μg/kg for the first, second and third target dose, respectively.

18. The T-cell engaging protein according to item 11, wherein the amount of all target doses is identical, and the amount is about 1080 μg/kg for the first, second and third target dose, respectively.

19. The T-cell engaging protein according to item 11, wherein the amount of all target doses is identical, and the amount is about 1530 μg/kg for the first, second and third target dose, respectively.

20. The T-cell engaging protein for use according to any one of items 1 to 10, the method further comprising a first treatment cycle following said step-in cycle, said first treatment cycle comprising the steps of:
administering to a subject a first target dose of the T-cell engaging protein, said first target dose ranging from about 5 mg to about 70 mg,
administering to the subject a second dose of the T-cell engaging protein, said second dose ranging from about 5 mg to about 70 mg, and
administering to the subject a third dose of the T-cell engaging protein, said third dose ranging from about 5 mg to about 70 mg,
   wherein optionally the amount of all three target doses is identical.

21. The T-cell engaging protein according to item 20, wherein the amount of all three target doses is identical and the amount ranges from about 5 mg to about 10 mg, preferably being about 10 mg, for the first, second and third target dose, respectively.

22. The T-cell engaging protein according to item 20, wherein the amount of all target doses is identical, and the amount is about 60 mg for the first, second and third target dose, respectively.

23. The T-cell engaging protein for use according to item 20, wherein at least one of said first, second or third target doses of said first treatment cycle comprises more than 30 mg.

24. The T-cell engaging protein for use according to item 20, wherein at least one of said first, second or third target doses of the first treatment cycle is about 5 mg, about 10 mg, about 20 mg, about 30 mg, about 40 mg, about 50 mg, about 60 mg, or about 70 mg, optionally about 10 mg or about 60 mg.

25. The T-cell engaging protein for use according to item 24, wherein at least one of said first, second or third target doses of the first treatment cycle is about 10 mg.

26. The T-cell engaging protein for use according to item 24, wherein at least one of said first, second or third target doses of the first treatment cycle is about 60 mg.

27. The T-cell engaging protein for use according to any one of items 11 to 26, wherein said first treatment cycle is not repeated or said first treatment cycle is repeated at least once.

28. The T-cell engaging protein for use according to any one of items 11 to 27, wherein said first, second and third target doses are administered within three weeks of said first treatment cycle, wherein optionally said first target dose is administered on day 1, the second target dose is administered on day 8 and the third target dose is administered on day 15 of said first treatment cycle, wherein optionally said first treatment cycle has a duration of three weeks.

29. The T-cell engaging protein for use according to any one of items 11 to 28, wherein said first treatment cycle is followed by a second treatment cycle, said second treatment cycle comprising the step of or consisting of the step:
administering to the subject a target dose of said T-cell engaging protein, said target dose ranging from about 80 μg/kg of the subject's body weight to about 1600 μg/kg of the subject's body weight;
   wherein said second treatment cycle is optionally repeated at least once.

30. The T-cell engaging protein according to item 29, wherein the amount of said target dose is about 90 μg/kg.

31. The T-cell engaging protein according to item 29, wherein the amount of said target dose is about 180 μg/kg.

31. The T-cell engaging protein according to item 29, wherein the amount of said target dose is about 270 μg/kg.

32. The T-cell engaging protein according to item 29, wherein the amount of said target dose is about 360 μg/kg.

33. The T-cell engaging protein according to item 29, wherein the amount of said target dose is about 540 μg/kg.

34. The T-cell engaging protein according to item 29, wherein the amount of said target dose is about 720 μg/kg.

35. The T-cell engaging protein according to item 29, wherein the amount of said target dose is about 1080 μg/kg.

36. The T-cell engaging protein according to item 29, wherein the amount of said target dose is about 1530 μg/kg.

37 The T-cell engaging protein for use according to any one of items 11 to 28, wherein said the first treatment cycle is followed by a second treatment cycle, said second treatment cycle comprising the step of or consisting of the step:
administering to the subject a target dose of the T-cell engaging protein, said target dose ranging from about 5 mg to about 70 mg;
   wherein said second treatment cycle is optionally repeated at least once.

38. The T-cell engaging protein according to item 37, wherein the amount of said target dose is about 10 mg.

39. The T-cell engaging protein according to item 20, wherein the amount said target dose is about 60 mg.

40. The T-cell engaging protein for use according to item 28 or 39, wherein said second treatment cycle has a total duration of three weeks and/or said target dose is administered on day 1 of said three weeks.

41. The T-cell engaging protein for use according to any one of items 23 to 40, wherein said first treatment cycle directly follows said step-in cycle; and/or said second treatment cycle directly follows said first treatment cycle.

42. The T-cell engaging protein for use according to any one of claims 1 to 9, wherein said Neuroendocrine Neoplasm (NEN) is a Neuroendocrine Carcinoma (NEC), preferably selected from the group consisting of Lung-NEC such as LCNEC of the lung, Extrapulmonary (ep) NEC including NEC with unknown primary site, small cell lung cancer (SCLC) including extensive-stage (ES)-SCLC, and limited-stage (LS) SCLC; or said NEN is a Neuroendocrine Tumor (NET) such as grade 3 NET.

43. The T-cell engaging protein for use according to any one of the preceding items, wherein a premedication comprising a non-steroidal anti-inflammatory drug, such as acetaminophen, an anti-histaminicum and/or a steroid, preferably a corticosteroid such as prednisolone or dexamethasone, prior to at least one of the doses, optionally prior to each dose.

44. The T-cell engaging protein for use according to any one of the preceding items, wherein the subject has been treated with a chemotherapy involving administration of a cytostatic agent prior to the step-in cycle.

45. The T-cell engaging protein for use according to any one of the preceding items, wherein an occurrence of cytokine release syndrome is to be treated with an anti-cytokine release syndrome agent, preferably selected from the group consisting of a corticosteroid, an IL-6 agonist and an IL-6R antagonist.

46. The T-cell engaging protein for use according to any one of the preceding items, wherein said T-cell engaging protein comprises immunoglobulin (Ig) domains, preferably 12 Ig domains.

47. The T-cell engaging protein for use according to item 23, wherein said Ig domains are comprised in two polypeptide chains, wherein each polypeptide chain comprises six Ig domains.

48. The T-cell engaging protein for use according to any one of the preceding items, wherein either antigen binding unit comprises six complementarity determining regions (CDRs).

49. The T-cell engaging protein for use according to item 48, wherein said T-cell engaging protein comprises a first antigen binding unit specifically binding to DLL3, comprising light chain CDRs comprising the amino acid sequences of SEQ ID NO:13 (CDR1), SEQ ID NO:14 (CDR2) and SEQ ID NO:15 (CDR3) and heavy chain CDRs comprising the amino acid sequences of SEQ ID NO:16 (CDR1), SEQ ID NO:17 (CDR2) and SEQ ID NO:18 (CDR3) and a second antigen binding unit specifically binding to CD3 comprising light chain CDRs comprising the amino acid sequences of SEQ ID NO:55 (CDR1), SEQ ID NO:56 (CDR2) and SEQ ID NO:57 (CDR3) and heavy chain CDRs comprising the amino acid sequences of SEQ ID NO:58 (CDR1), SEQ ID NO:59 (CDR2) and SEQ ID NO:60 (CDR3).

50. The T-cell engaging protein for use according to item 49, wherein said T-cell engaging protein comprises (i) a first antigen binding unit specifically binding to DLL3 comprising a light chain variable domain comprising the amino acid sequence of SEQ ID NO:41 and heavy chain variable domain comprising the amino acid sequence of SEQ ID NO:42 and (ii) a second antigen binding unit specifically binding to CD3 comprising a light chain variable domain comprising the amino acid sequence of SEQ ID NO:67 and a heavy chain variable domain comprising the amino acid sequence of SEQ ID NO:68.

51. The T-cell engaging protein for use according to item 50, wherein said T-cell engaging protein comprises a first single chain Fab forming a first antigen binding unit comprising the sequence of SEQ ID NO:51 and a second single chain Fab comprising the sequence of SEQ ID NO:71 forming a second antigen binding unit.

52. The T-cell engaging protein for use according to any one of the preceding items, wherein said T-cell engaging protein comprises of a first polypeptide chain specific for DLL3 comprising an amino acid sequence of SEQ ID NO:75 and a second polypeptide chain specific for CD3 comprising the amino acid sequence of SEQ ID NO:79.

53. The T-cell engaging protein for use according to any one of the preceding items, wherein said T-cell engaging protein consists of a first polypeptide chain specific for DLL3 comprising an amino acid sequence of SEQ ID NO:75 and a second polypeptide chain specific for CD3 comprising the amino acid sequence of SEQ ID NO:79.

54. The T-cell engaging protein for use according to any one of the preceding items, wherein essentially no cytokine release syndrome occurs after said initial and/or said elevated dose of said step-in cycle in the subject.

55. The T-cell engaging protein for use according to any one of the preceding items, wherein said T-cell engaging protein is the only anti-cancer agent to be administered.

56. The T-cell engaging protein for use according to any one of the preceding items, wherein at least one further anti-cancer agent is to be administered, said further anti-cancer agent preferably being a chemotherapeutic agent and/or an immune-checkpoint inhibitor such as an anti-PD-1 antibody or an anti-PD-L1 antibody.

57. The T-cell engaging protein for use according to any one of items 1, 2 or 4-56, wherein said initial dose ranges from about 5 μg/kg of the subject's body weight to about 15 μg/kg of the subject's body weight; said elevated dose ranges from about 55 μg/kg of the subject's body weight to about 95 μg/kg of the subject's body weight, and optionally said elevated dose is about 90 μg/kg of the subject's body weight; and said further dose ranges from about 5 mg to about 15 mg or about about 50 mg to about 70 mg.

58. The T-cell engaging protein for use according to item 57,
wherein said initial dose is about 10 μg/kg;
said elevated dose is about 90 μg/kg; and
said further dose is about 10 mg.

59. The T-cell engaging protein for use according to item 57,
wherein said initial dose is about 10 μg/kg;
said elevated dose is about 60 μg/kg; and
said further dose is about 10 mg.

60. The T-cell engaging protein for use according to item 57,
wherein said initial dose is about 10 μg/kg;
said elevated dose is about 60 μg/kg; and
said further dose is about 60 mg.

61. The T-cell engaging protein for use according to item 57,
wherein said initial dose is about 10 μg/kg;
said elevated dose is about 10 mg; and
said further dose is about 60 mg.

62. A T-cell engaging protein comprising a first antigen binding unit that specifically binds to DLL3 and a second antigen binding unit that specifically binds to CD3 for use in a method of treatment of a DLL3-positive cancer or a cancer selected from a Neuroendocrine Neoplasm (NEN), Merkel cell carcinoma (MCC), medullary thyroidal carcinoma and glioma, the method comprising a step-in cycle, the step-in cycle comprising the steps of:
administering to the subject an initial dose, an elevated dose, which is elevated as compared to said initial dose, and a further dose of said T-cell engaging protein, wherein a ratio between said initial, elevated and further dose per kilogram of the subject's body weight is about 1: about 3: about 9.

63. A T-cell engaging protein comprising a first antigen binding unit that specifically binds to DLL3 and a second antigen binding unit that specifically binds to CD3 for use in a method of treatment of a DLL3-positive cancer or a cancer selected from a Neuroendocrine Neoplasm (NEN), Merkel cell carcinoma (MCC), medullary thyroidal carcinoma and glioma,
the method comprising a step-in cycle, the step-in cycle comprising the steps of:
   administering to a subject an initial dose of said T-cell engaging protein, said initial dose ranging from about 5 μg/kg of the subject's body weight to about 15 μg/kg of the subject's body weight,
   administering to the subject an elevated dose, which is elevated as compared to said initial dose, of said T-cell engaging protein, the elevated dose ranging from about 20 μg/kg of the subject's body weight to about 40 μg/kg of the subject's body weight,
   administering to the subject a further dose of said T-cell engaging protein, said further dose ranging from about 80 μg/kg of the subject's body weight to about 1600 μg/kg of the subject's bodyweight.

64. The T-cell engaging protein for use according to item 62 or 63, wherein said further dose is equal to a target dose, said target dose being the dose to be administered during a first treatment cycle which optionally directly follows said step-in cycle.

65. The T-cell engaging protein for use according to any one of items 62 to 64, wherein said initial dose, said elevated dose and/or said further is/are administered once; or
said initial dose, said elevated dose and/or said further is/are administered more than once, optionally 2 to 10 times,
wherein optionally the number of administrations is determined based on presence and/or degree of a cytokine release syndrome (CRS) and/or of an immune effector cell-associated neurotoxicity syndrome (ICANS),
and wherein further optionally said elevated dose follows the initial dose only if said CRS, to the extent determined, is rated grade 1 or less in terms of NCI CTC grade and/or if said ICANS, to the extend determined, is rated grade 1 or less, and/or wherein further optionally
further optionally said further dose follows the elevated dose only if said CRS, to the extent determined, is rated grade 1 or less in terms of NCI CTC grade and/or if said ICANS, to the extend determined, is rated grade 1 or less.

66. The T-cell engaging protein for use according to any one of items 62 to 65, wherein said initial dose and/or said elevated dose is administered three times to ten times within the step-in cycle.

67. The T-cell engaging protein for use according to any one of items 62 to 66, wherein said initial dose is administered three times within three weeks, optionally on day 1, day 8 and day 15 of the three weeks.

68 The T-cell engaging protein for use according to any one of items 62 to 67, wherein said step-in cycle has a duration of at least three weeks.

69. The T-cell engaging protein for use according to any one of items 62 to 68, wherein said initial, elevated and further doses are administered within three weeks, said initial dose being administered once, wherein optionally said initial dose is administered on day 1, said elevated dose is administered on day 8 and the said further dose is administered on day 15 of the step-in cycle.

70. The T-cell engaging protein for use according to any one of items 62 to 69, wherein said initial dose is about 10 μg/kg of the subject's body weight;
said elevated dose is about 30 μg/kg of the subject's body weight; and
said further dose is about is 90 μg/kg of the subject's body weight.

71. The T-cell engaging protein for use according to any one of items 62 to 70; the method further comprising a first treatment cycle following the step-in cycle, said first treatment cycle comprising the steps of:
administering to a subject a first target dose of said T-cell engaging protein, said first target dose ranging from about 80 μg/kg of the subject's body weight to about 1600 μg/kg of the subject's body weight,
administering to the subject a second target dose of the T-cell engaging protein, said second target dose ranging from about 80 μg/kg of the subject's body weight to about 1600 μg/kg of the subject's body weight, and
administering to the subject a third target dose of the T-cell engaging protein, said third target dose ranging from about 80 μg/kg of the subject's body weight to about 1600 μg/kg of the subject's bodyweight,
   wherein optionally the amount of all three target doses is identical.

72. The T-cell engaging protein according to item 71, wherein the amount of all three target doses is identical and the amount is about 90 μg/kg for the first, second and third target dose, respectively.

73. The T-cell engaging protein according to item 71, wherein the amount of all target doses is identical, and the amount is about 270 μg/kg for the first, second and third target dose, respectively.

74. The T-cell engaging protein according to item 71, wherein the amount of all target doses is identical, and the amount is about 360 μg/kg for the first, second and third target dose, respectively.

75. The T-cell engaging protein according to item 71, wherein the amount of all target doses is identical, and the amount is about 540 μg/kg for the first, second and third target dose, respectively.

76. The T-cell engaging protein according to item 71, wherein the amount of all target doses is identical, and the amount is about 720 μg/kg for the first, second and third target dose, respectively.

77. The T-cell engaging protein according to item 71, wherein the amount of all target doses is identical, and the amount is about 1080 μg/kg for the first, second and third target dose, respectively.

78. The T-cell engaging protein for use according to any one of items 62 to 70, the method further comprising a first treatment cycle following the step-in cycle, the first treatment cycle comprising the steps of:
administering to a subject a first target dose of the T-cell engaging protein, said first target dose ranging from about 5 mg to about 70 mg,
administering to the subject a second target dose of the T-cell engaging protein, said second target dose ranging from about 5 mg to about 70 mg, and
administering to the subject a third target dose of the T-cell engaging protein, said third dose ranging from about 5 mg to about 70 mg,
   wherein optionally the amount of all three target doses is identical.

79. The T-cell engaging protein according to item 78, wherein the amount of said target dose is about 10 mg.

80. The T-cell engaging protein according to item 78, wherein the amount said target dose is about 60 mg.

81. The T-cell engaging protein for use according to item 78, wherein at least one of the first, second or third target doses of the first treatment cycle comprises more than 30 mg.

82. The T-cell engaging protein for use according to item 78, wherein at least one of said first, second or third target doses of the first treatment cycle is about 5 mg, about 10 mg, about 20 mg, about 30 mg, about 40 mg, about 50 mg, about 60 mg, or about 70 mg, optionally about 10 mg or about 60 mg.

83. The T-cell engaging protein for use according to item 82, wherein at least one of said first, second or third target doses of the first treatment cycle is about 10 mg.

84. The T-cell engaging protein for use according to item 83, wherein at least one of said first, second or third target doses for the first, second or third target doses of the first treatment cycle is about 60 mg.

85. The T-cell engaging protein for use according to any one of items 62 to 84, wherein said first, second and third target doses are administered within three weeks of the first treatment cycle, wherein optionally said first target dose is administered on day 1, said second target dose is administered on day 8 and said third target dose is administered on day 15 of said first treatment cycle, wherein optionally said first treatment cycle has a duration of three weeks.

86. The T-cell engaging protein for use according to any one of items 62 to 85, wherein said first treatment cycle is followed by a second treatment cycle, said second treatment cycle comprising the step of or consisting of the step:
administering to the subject a target dose of said T-cell engaging protein, said target dose ranging from about 80 μg/kg of the subject's body weight to about 1600 μg/kg of the subject's body weight;
wherein said second treatment cycle is optionally repeated at least once.

87. The T-cell engaging protein according to item 86, wherein the amount of said target dose is about 90 μg/kg.

88. The T-cell engaging protein according to item 86, wherein the amount of said target dose is about 270 μg/kg.

89. The T-cell engaging protein according to item 86, wherein the amount of said target dose is about 360 μg/kg.

90. The T-cell engaging protein according to item 86, wherein the amount of said target dose is about 540 μg/kg.

91. The T-cell engaging protein according to item 86, wherein the amount of said target dose is about 720 μg/kg.

92. The T-cell engaging protein according to item 86, wherein the amount of said target dose is about 1080 μg/kg.

93. The T-cell engaging protein for use according to any one of items 62 to 86, wherein said first treatment cycle is followed by a second treatment cycle, said second treatment cycle comprising the step of or consisting of the step:
administering to the subject a target dose of said T-cell engaging protein, said target dose ranging from about 5 mg to about 70 mg;
wherein said second treatment cycle is optionally repeated at least once.

94. The T-cell engaging protein for use according to item 93, wherein said target dose is about 10 mg.

95. The T-cell engaging protein for use according to item 93, wherein said target dose is about 60 mg.

96. The T-cell engaging protein for use according to item 86 or 95, wherein said second treatment cycle has a total duration of three weeks and/or said target dose is administered on day 1 of the three weeks.

97. The T-cell engaging protein for use according to any one of items 62 to 96, wherein the first treatment cycle directly follows said step-in cycle; and/or the second treatment cycle directly follows said first treatment cycle.

98. The T-cell engaging protein for use according to any one of items 62 to 97, wherein said Neuroendocrine Neoplasm (NEN) is a Neuroendocrine Carcinoma (NEC), preferably selected from the group consisting of Lung-NEC such as LCNEC of the lung, Extrapulmonary (ep) NEC including NEC with unknown primary site, small cell lung cancer (SCLC) including extensive-stage (ES)-SCLC, and limited-stage (LS) SCLC; or said NEN is a Neuroendocrine Tumor (NET) such as grade 3 NET.

99. The T-cell engaging protein for use according to any one of items 62 to 98, wherein a premedication comprising a non-steroidal anti-inflammatory drug, such as acetaminophen, an anti-histaminicum and/or a steroid, such as prednisolone or dexamethasone, prior to at least one of the doses, optionally prior to each dose.

100. The T-cell engaging protein for use according to any one of items 62 to 99, wherein the subject has been treated with a chemotherapy involving administration of inhibitors of cell growth and/or division prior to the step-in cycle.

101. The T-cell engaging protein for use according to any one of items 62 to 100, wherein an occurrence of cytokine release syndrome is to be treated with an anti-cytokine release syndrome agent, preferably selected from the group consisting of a corticosteroid, an IL-6 antagonist and an IL-6R antagonist.

102. The T-cell engaging protein for use according to any one of items 62 to 101, wherein said T-cell engaging protein comprises immunoglobulin (Ig) domains, preferably 12 Ig domains.

103. The T-cell engaging protein for use according to item 102, wherein said Ig domains are comprised in two polypeptide chains, wherein each polypeptide chain comprises six Ig domains.

104. The T-cell engaging protein for use according to any one of items 62 to 103, wherein either antigen binding unit comprises six complementarity determining regions (CDRs).

105. The T-cell engaging protein for use according to item 104, wherein said T-cell engaging protein comprises a first antigen binding unit specifically binding to DLL3, comprising light chain CDRs comprising the amino acid sequences of SEQ ID NO:13 (CDR1), SEQ ID NO:14 (CDR2) and SEQ ID NO:15 (CDR3) and heavy chain CDRs comprising the amino acid sequences of SEQ ID NO:16 (CDR1), SEQ ID NO:17 (CDR2) and SEQ ID NO:18 (CDR3) and a second antigen binding unit specifically binding to CD3 comprising light chain CDRs comprising the amino acid sequences of SEQ ID NO:55 (CDR1), SEQ ID NO:56 (CDR2) and SEQ ID NO:57 (CDR3) and heavy chain CDRs comprising the amino acid sequences of SEQ ID NO:58 (CDR1), SEQ ID NO:59 (CDR2) and SEQ ID NO:60 (CDR3).

106. The T-cell engaging protein for use according to item 105, wherein said T-cell engaging protein comprises (i) a first antigen binding unit specifically binding to DLL3 comprising a light chain variable domain comprising the amino acid sequence of SEQ ID NO:41 and heavy chain variable domain comprising the amino acid sequence of SEQ ID NO:42 and (ii) a second antigen binding unit specifically binding to CD3 comprising a light chain variable domain comprising the amino acid sequence of SEQ ID NO:67 and a heavy chain variable domain comprising the amino acid sequence of SEQ ID NO:68.

107. The T-cell engaging protein for use according to item 106, wherein said T-cell engaging protein comprises a first single chain Fab forming a first antigen binding unit comprising the sequence of SEQ ID NO:51 and a second single chain Fab comprising the sequence of SEQ ID NO:71 forming a second antigen binding unit.

108. The T-cell engaging protein for use according to any one of items 62 to 107, wherein said T-cell engaging protein comprises a first polypeptide chain specific for DLL3 comprising an amino acid sequence of SEQ ID NO:75 and a second polypeptide chain specific for CD3 comprising the amino acid sequence of SEQ ID NO:79.

109. The T-cell engaging protein for use according to any one of items 62 to 108, wherein said T-cell engaging protein consists of a first polypeptide chain specific for DLL3 comprising an amino acid sequence of SEQ ID NO:75 and a second poly-peptide chain specific for CD3 comprising the amino acid sequence of SEQ ID NO:79.

110. The T-cell engaging protein for use according to any one of items 62 to 109, wherein essentially no cytokine release syndrome occurs after said elevated dose of said step-in cycle in the subject.

111. The T-cell engaging protein for use according to any one of items 62 to 110, wherein said T-cell engaging protein is the only anti-cancer agent to be administered.

112. The T-cell engaging protein for use according to any one of items 62 to 111, wherein at least one further anti-cancer agent is to be administered, said further anti-cancer agent preferably being a chemotherapeutic agent and/or an immune-checkpoint inhibitor such as an anti-PD-1 antibody or an anti-PD-L1 antibody.

113. The T-cell engaging protein for use according to any one of the preceding items, wherein the T-cell engaging protein is administered by an administration route selected from the group consisting of parenteral, subcutaneous, intraperitoneal, intrapulmonary, intranasal, and/or intralesional administration 114. The T-cell engaging protein for use according to item 113, wherein the T-cell engaging protein is administered by parenteral administration, parenteral administration including administrations selected from the group consisting intramuscular, intravenous, intraarterial, intraperitoneal, or subcutaneous administration.

115. The T-cell engaging protein for use according to item 114, wherein the T-cell engaging protein is administered by intravenous (IV) infusion.

116. The T-cell engaging protein for use according to any one of the preceding items, wherein the subject is a human.

### EXAMPLES

### Example 1: Phase I Study of BI 764532

The study described herein is a first-in-human, Phase I study of BI 764532 monotherapy in patients with DLL3 expressing SCLC, large cell NEC, NEC or small cell carcinomas of any other origin (NCT04429087).

The objectives of this study are to determine the maximum tolerated dose(s) (MTD) or the Recommended Dose for Expansion (RDE) and dosing regimen for BI 764532 in patients with SCLC or other NECs, and to evaluate safety, tolerability, pharmacokinetics (PK) and preliminary efficacy.

### Methods and Analysis

### Study Design

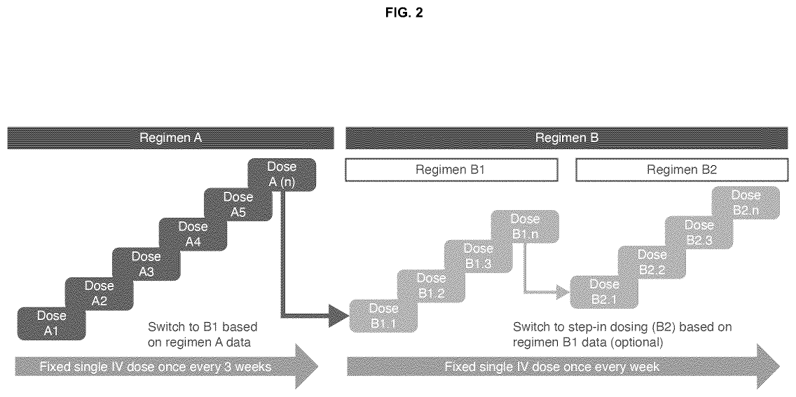

This Phase I study is a multicenter, open-label, non-randomized, dose-escalation study. In the Phase Ia period, BI 764532 is administered using different dosing regimens (FIG. 2):
Regimen A: target dose once every 3 weeks, i.e. on day 1 of a 3-week cycle,
Regimen B1: target dose once a week, i.e. on Days 1, 8 and 15 of a 3-week cycle,
Regimen B2: initial weekly step-in dose(s) followed by a target dose administered weekly i.e. on Days 1, 8 and 15 of a 3-week cycle

### Dosing Regimen A

Treatment cycles of 3 week (21 day) duration. BI 764532 monotherapy. Dosing regimen comprising once every three week (q3w) intravenous (i.v.) administration of the target (x ug/kg) dose in cycles 1-x (i.e. on day 1 of each 21 day cycle).

TABLE 3A
Regimen A:
One cycle duration = 3 weeks
Treatment cycle and following: target dose administered on day 1 of
each cycle (i.e. q3w) until progression or toxicity, maximum of 36 months

| | Treatment cycle and following |
|---|---|
| | d 1 |
| dose | Target dose: |
| | from 0.03 to 90 μg/kg: |
| | 0.03 μg/kg, 0.1 μg/kg, 0.3 μg/kg, |
| | 0.9 μg/kg, 2.5 μg/kg, 5 μg/kg, |
| | 10 μg/kg, 30 μg/kg, 90 μg/kg |

### Dosing Regimen B1

Treatment cycles of 3 week (21 day) duration. BI 764532 monotherapy. Dosing regimen comprising once every week (q1w) intravenous (i.v.) administration of the target (x ug/kg) dose in cycles 1 and following (i.e. on day 1, 8 and 15 of each 21 day cycle).

TABLE 3B
Regimen B1:
One cycle duration = 3 weeks
Treatment cycle and following: target dose administered
on day 1, 8 and 15 of each cycle (i.e. q1w) until
progression or toxicity, maximum of 36 months

| | Treatment cycle and following | | |
|---|---|---|---|
| | d 1 | d 8 | d 15 |
| dose | Target dose: | Target dose: | Target dose: |
| | from 0.9 to | from 0.9 to | from 0.9 to |
| | 30 μg/kg: | 30 μg/kg: | 30 μg/kg: |
| | 0.9 μg/kg, 10 μg/kg, | 0.9 μg/kg, 10 μg/kg, | 0.9 μg/kg, 10 μg/kg, |
| | 30 μg/kg | 30 μg/kg | 30 μg/kg |

### Dosing Regimen B2

Cycles of 3 week (21 day) duration. BI 764532 monotherapy. Dosing regimen comprising intravenous (i.v.) administration of a first step in dose (×1 ug/kg) in step-in cycle on day 1, a second step-in dose (×2 ug/kg) in step-in cycle on day 8, and a target dose (×3 ug/kg) in step-in cycle on day 15, followed by weekly i.v. administration of the target (x ug/kg) dose in all treatment cycles (i.e. on day 1, 8 and 15 of each 21 day cycle). A switch to Regimen B2 could be decided if ≥grade 2 toxicity occurred in regimen A or B1 after the first administration and this toxicity was determined to be due to CRS or related symptoms.

TABLE 3C
Regimen B2:
One cycle duration = 3 weeks
Step-in cycle: initial 3 doses on weekly intervals including 2 “step-in” doses
(d 1, d 8) and first target dose (d 15); target doses tested: 90 μg/kg 180 μg/kg,
270 μg/kg, 360 μg/kg, 540 μg/kg, 720 μg/kg, 1080 μg/kg, 1530 μg/kg)
Treatment cycles: Target doses (90 μg/kg 180 μg/kg, 270 μg/kg, 360 μg/kg,
540 μg/kg, 720 μg/kg, 1080 μg/kg, 1530 μg/kg) administered weekly
until progression or toxicity, maximum of 36 months

| | Step-in cycle | | | Treatment cycle 1 (repeated) | | |
|---|---|---|---|---|---|---|
| | d 1 | d 8 | d 15 | d 1 | d 8 | d 15 |
| dose | 30 ug/kg | 90 ug/kg | Target dose: | Target dose: | Target dose: | Target dose: |
| | | | from 90 to | from 90 to | from 90 to | from 90 to |
| | | | 1530 ug/kg | 1530 ug/kg | 1530 ug/kg | 1530 ug/kg |

Regimens are assessed sequentially, starting with Regimen A (FIG. 2). Patients will be treated until disease worsening or for a maximum of 36 months.

These schedules of administration were selected according to pharmacokinetic modelling based on preclinical data.

Regimen A was selected to avoid clinically significant drug accumulation beyond the first cycle.

Accordingly, it was considered appropriate to explore the safety of a single dose BI 764532 using a Bayesian Logistic Regression Model (BLRM) with an initial MTD period of 3 weeks.

Regimen B1 was initiated once data from Regimen A, including safety, pharmacokinetics, biomarker analysis, potential of drug accumulation and possible efficacy had been assessed.

Regimen B2 was initiated according to protocol when the observed adverse events (AEs), especially cytokine release syndrome (CRS) precluded further does escalation in Regimens A and B1. In regimens A and B1 the target dose was administered from the first administration and onwards (q3w or q1w); dose escalation in A and B1 was stopped at 90 and 30 μg/kg, respectively (safety findings at the initial 90 μg/kg administration did not allow further dose escalation despite premedication including steroids) and Regimen B2 (step-in dosing) was initiated with the aim to reduce the frequency and severity of adverse events at the initial administration(s), especially CRS and ICANS, and to reach higher therapeutic doses (target doses).

### Main Inclusion Criteria

Patients are adults (≥18 years) with locally advanced or metastatic tumors of the following histologies: SCLC; large cell neuroendocrine lung carcinoma; NEC or small cell carcinoma of any other origin. Patients have failed or are not eligible for available standard therapies and are previously received ≥1 line of chemotherapy (for SCLC platinum-based). Patients have an Eastern Cooperative Oncology Group performance status of 0 or 1, at least one evaluable lesion outside of the central nervous system (CNS), as defined by modified Response Evaluation Criteria In Solid Tumors (RECIST) version 1.1 criteria, adequate organ function, and have provided written informed consent.

### Study Endpoints

The primary endpoints of the Phase Ia period are: MTD in any studied regimen, defined as the highest dose with <25% risk of the true DLT rate being ≥33% during the MTD evaluation period, with separate MTDs determined in Regimen A and Regimen B; and the number of patients with DLTs in the MTD evaluation period.

The secondary endpoints of the Phase Ia period are objective response based on RECIST 1.1 criteria in patients with measurable disease, and the PK parameters maximum measured concentration of BI 764532 (Cmax) and the area under the concentration-time curve of the analyte over a uniform dosing interval τ (AUCτ). Further endpoints include DLL3 expression; tumor pathology and grade; immunogenic response; pharmacodynamic biomarker modulation in tumor tissue and blood; the number of patients with DLT during the entire treatment period; progression-free survival and duration of response; changes in soluble DLL3 in plasma over time; changes in circulating tumor cell counts over time (in SCLC patients only); and additional PK parameters.

### Statistical Analysis

### Determination of the MTD

For each dosing regimen, a separate BLRM employing the EWOC principle is used during the escalation phase for the selection of the dose levels and the estimation of the MTD, where applicable (Babb J, Rogatko A, Zacks S. Cancer phase I clinical trials: efficient dose escalation with overdose control. Stat Med 17(10), 1103-1120 (1998); Neuenschwander B, Branson M, Gsponer T. Critical aspects of the Bayesian approach to phase I cancer trials. Stat Med 27(13), 2420-2439 (2008)). The estimated probability of a DLT at each dose level from the model is summarized using the following intervals: under toxicity, 0.00-0.16; targeted toxicity, 0.16-0.33; and over toxicity, 0.33-1.00. Applying the EWOC criterion, it should be unlikely (less than 25% probability) that the DLT rate at that dose exceeds 0.33.

The MTD may be considered reached if one of the following criteria is fulfilled: the posterior probability of the true DLT rate in the target toxicity interval of the MTD is above 0.5, or at least 15 patients have been treated in the dose escalation phase of the trial, of which at least six have been treated at the MTD.

Safety Data from Phase I Study Across Regimens a, B1 and B2

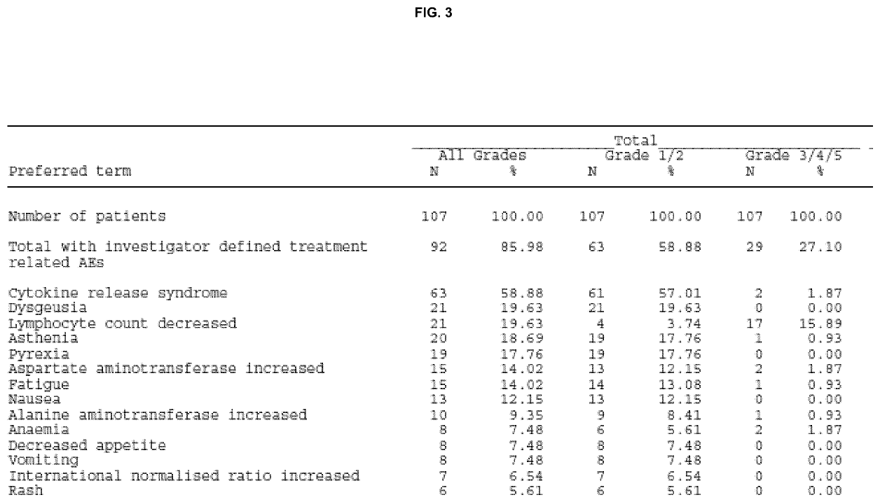

Safety data from the patients treated in the regimens A, B1 and B2 described in Example 3 above were collected using the CTCAE-version 5. The observed drug-related adverse events (AEs) are shown in FIG. 3.

As expected from the BI 764532 Mode of Action (MoA) the most frequent drug-related AE is CRS which occurs in approximately 60% of the patients (all grades), most of the CRS events were low grade (CTCAE 1-2) and only approx. 2% of the patients experienced CRS grade 3 or higher.

Based on MoA and other published data for other TcE treatments as well neurological AEs, especially ICANS, could be expected as well.

### Results of Phase I Study

### A1

For Regimen A (see Table 3A above) the starting dose of BI 764532 was 0.03 μg/kg, based on a pharmacologically active dose, estimated from the 20% effect concentrations of the most sensitive in vitro pharmacology assay. For BI 764532 dose escalation the following provisional dose levels were planned per protocol: 0.1, 0.3, 0.9, 2.5, 5, 10, 20, 40 and 80 μg/kg. The trial protocol allowed for lower or higher doses to be investigated as long as they fulfil the escalation with overdose control (EWOC) principle. Application of the EWOC principle means that any dose tested should be unlikely (<25% posterior probability) to result in a DLT rate of ≥33%.

Based on reported safety per dose group the BI 764532 dose was escalated over a total of 9 dose levels (0.03 μg/kg, 0.1 μg/kg, 0.3 μg/kg; 0.9 μg/kg, 2.5 μg/kg, 5 μg/kg, 10 μg/kg, 30 μg/kg, 90 μg/kg). CRS (all grades) per dose level was reported in Regimen A at the following frequencies: 0% at 0.03 μg/kg, 0% at 0.1 μg/kg, 0% at 0.3 μg/kg; 33% at 0.9 μg/kg, 33% at 2.5 μg/kg, 33% at 5 μg/kg, 33% at 10 μg/kg, 33% 30 μg/kg, 100% at 90 μg/kg. These results indicate that frequency of CRS increases with dose (dose safety correlation). At the dose of 90 μg/kg all pts experienced CRS (6 out of 6, 100%) in 2 cases the CRS was CTCAE grade 2 (33%). All pts treated at the dose of 90 μg/kg experienced CRS at the first BI 764532 administration; at the 2^{nd} administration 2 out of 6 pts experienced CRS and at subsequent administrations (≥3) only one patient was reported with CRS. These results indicate that the CRS frequency decreases with repeated dosing. Furthermore, 3 pts (50% of pts) were reported with ICANS at BI 764532 90 μg/kg, ICANS was grade 2 in 2 cases and grade 3 in one case. Based on these safety findings it was concluded that 90 μg/kg in Regimen A was the upper limit where the dose can be safely escalated, and it was decided to stop dose escalation in Regimen A.

Tumor response was assessed in all patients enrolled. No response (CR or PR) by RECIST 1.1 was reported at BI 764532 dose levels of 0.03 μg/kg, 0.1 μg/kg, 0.3 μg/kg; 0.9 μg/kg, 2.5 μg/kg, 5 μg/kg, 10 μg/kg and 30 μg/kg. At 90 μg/kg a PR was reported in two patients. Accordingly, in Regimen A the 90 μg/kg dose was the only dose with a signal of clinical efficacy (FIG. 5A).

### B1

Dose escalation steps in Regimen B1 (see Table 3B): Based on reported safety per dose group in Regimen A it was decided to escalate in Regimen B1 the BI 764532 dose from 0.9 μg/kg to 30 μg/kg over a total of 3 dose levels (0.9 μg/kg, 10 μg/kg and 30 μg/kg). CRS (all grades) per dose level was reported in Regimen B1 at the following frequencies: 0% at 0.9 μg/kg, 33% at 10 μg/kg, 50% at 30 μg/kg. Most of the CRS events occurred at the initial BI 764532 administration. The B1 data indicate that with q1w administration similar doses (per administration) are tolerable as with q3w dosing, i.e., the dose per administration rather than the cumulative dose per cycle seemed to determine tolerability. Dose escalation in Regimen B1 was stopped when the dose level 30 μg/kg was completed based on results from Regimen A indicating that further dose escalation in B1 to 90 μg/kg would not have been tolerable.

Tumor response was assessed in all patients enrolled. No response (CR or PR) by RECIST 1.1 was reported in Regimen B1 at any BI 764532 dose level. Accordingly, in Regimen B1 no signal of clinical efficacy was detected.

### B2

Rationale for B2: In regimens A and B1
dose escalation was stopped at 90 and 30 μg/kg, respectively, because of safety findings at the initial 90 μg/kg administration that did not allow further dose escalation despite premedication including steroids.
CRS frequency at the initial administration increased with dose (dose safety correlation)
CRS frequency decreased with repeated dosing

These results led to the conclusion that starting with a lower dose at the initial administration and increasing the dose at subsequent administrations (step-in dosing) should improve tolerability and might allow to reach target doses>90 μg/kg. Regimen B2 (step-in dosing, see Table 3C) was accordingly implemented aiming to reduce the frequency and severity of adverse events, such as CRS and ICANS and to reach higher therapeutic doses (target dose). For Regimen B2 the first administration (d1) was chosen to be 30 μg/kg and the second administration (d8) was chosen to be 90 μg/kg based on safety data generated in Regimens A and B1. The third administration (d15) in B2 was defined to be the target dose. During Regimen B2 the initial administration of 30 μg/kg and second administration of 90 μg/kg remained fixed and target dose was escalated.

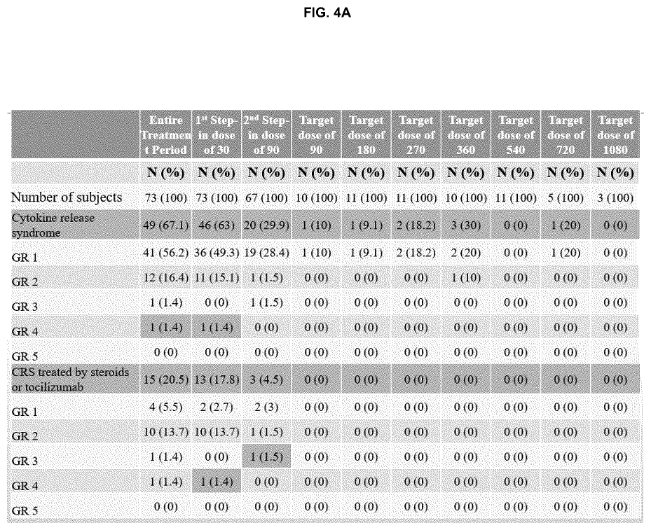

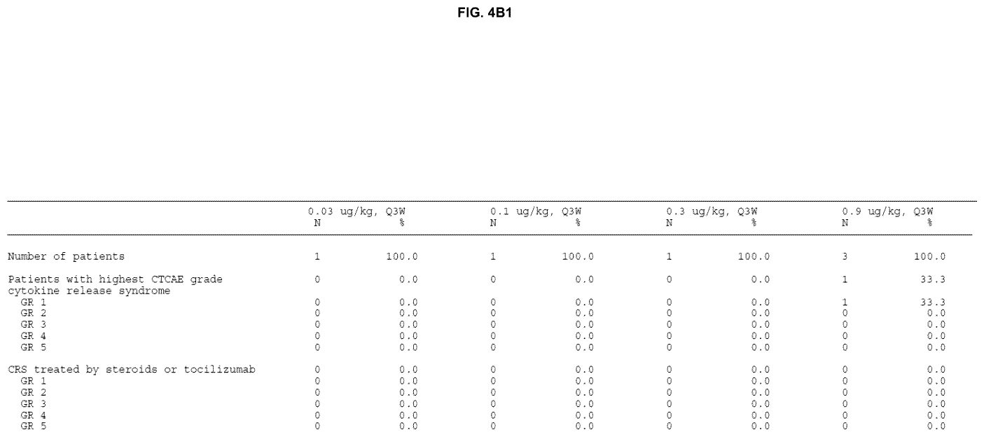

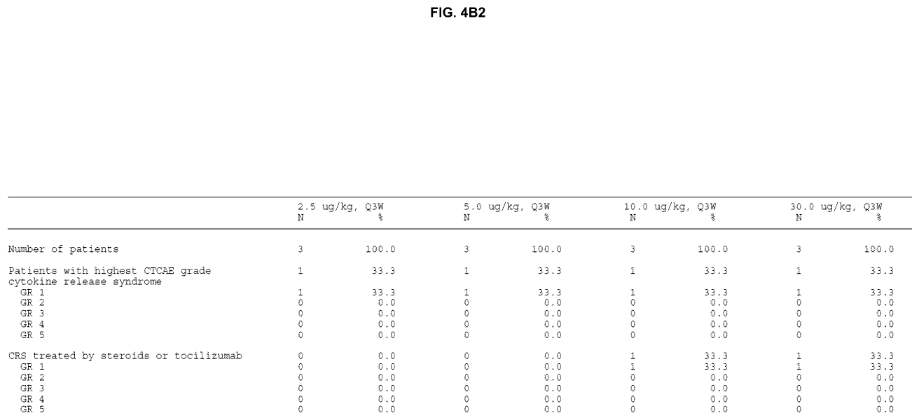

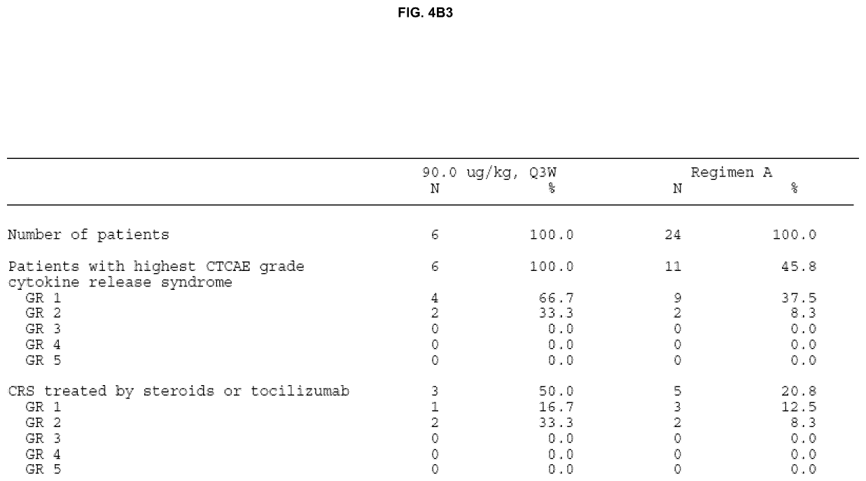

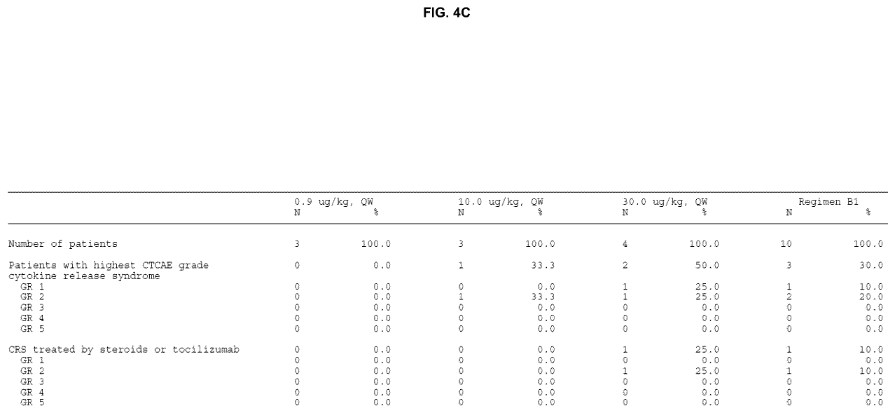

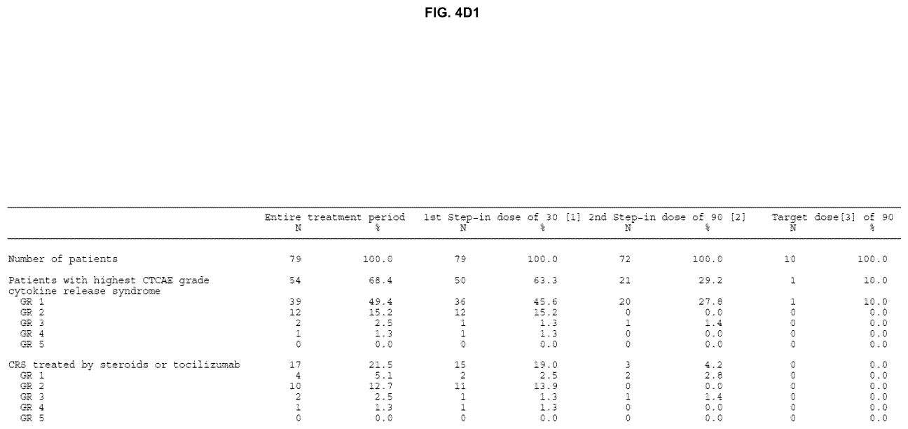

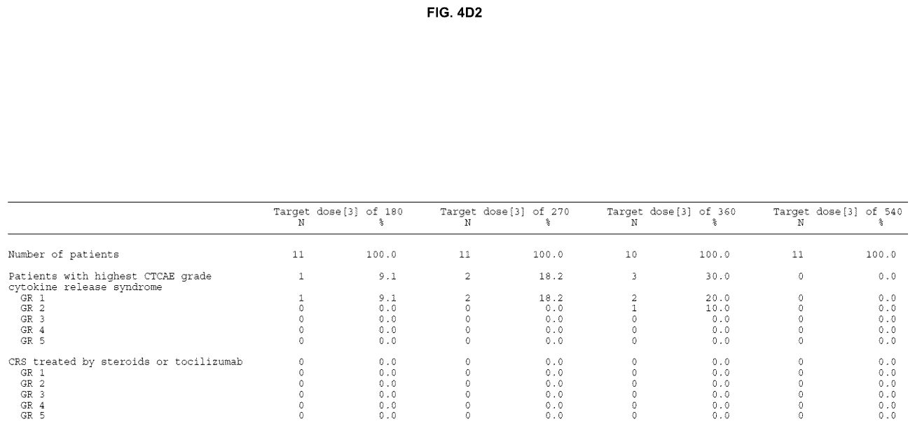

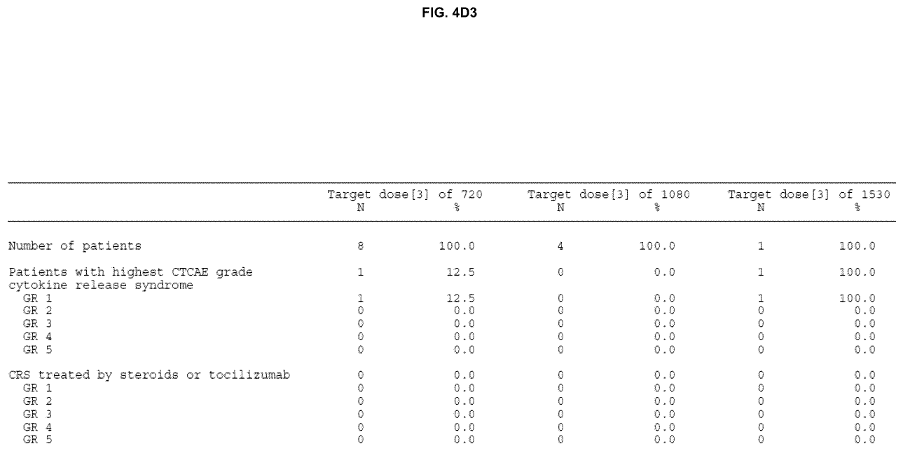

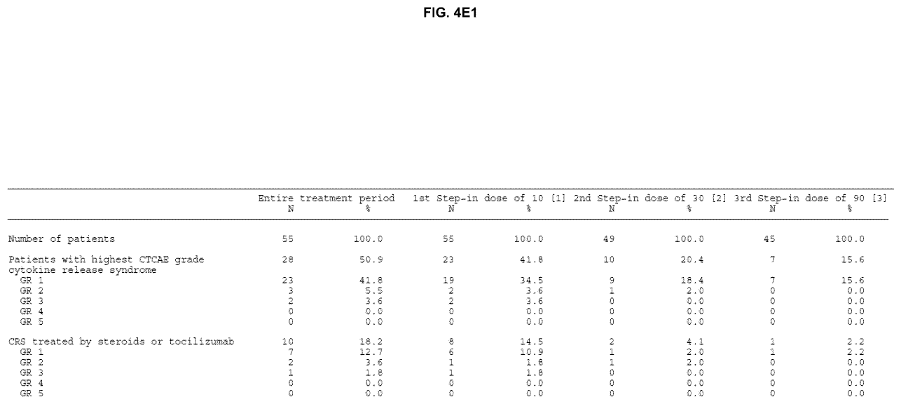

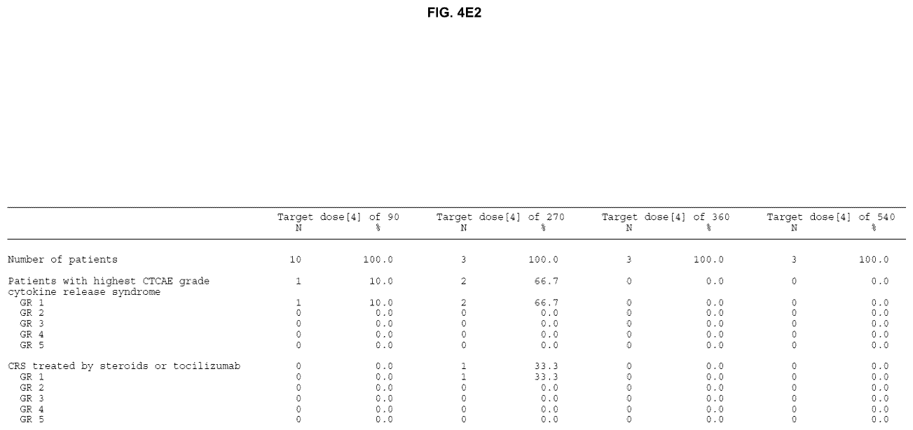

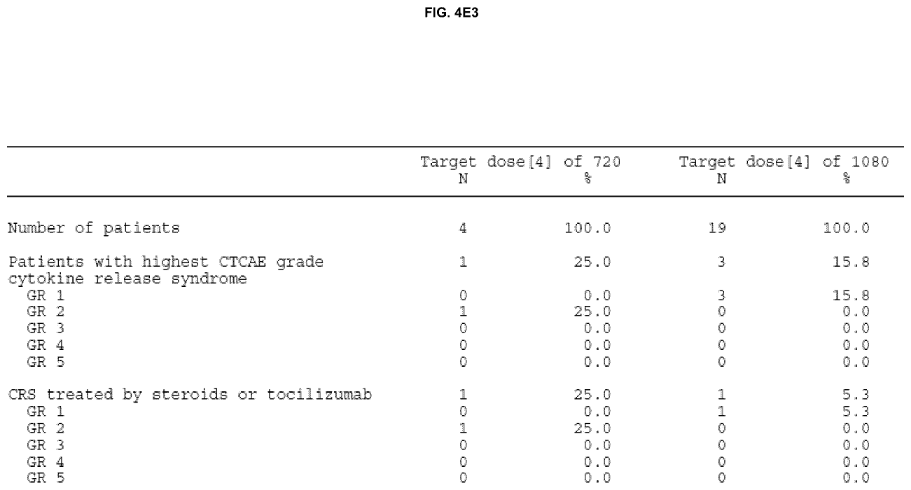

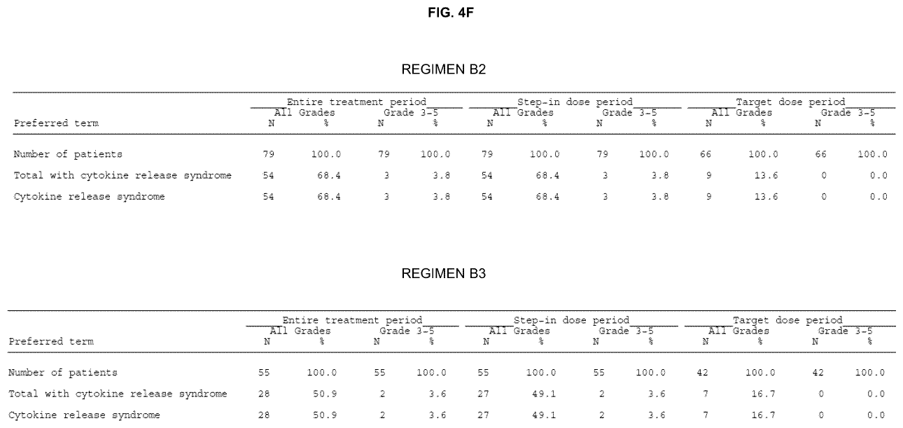

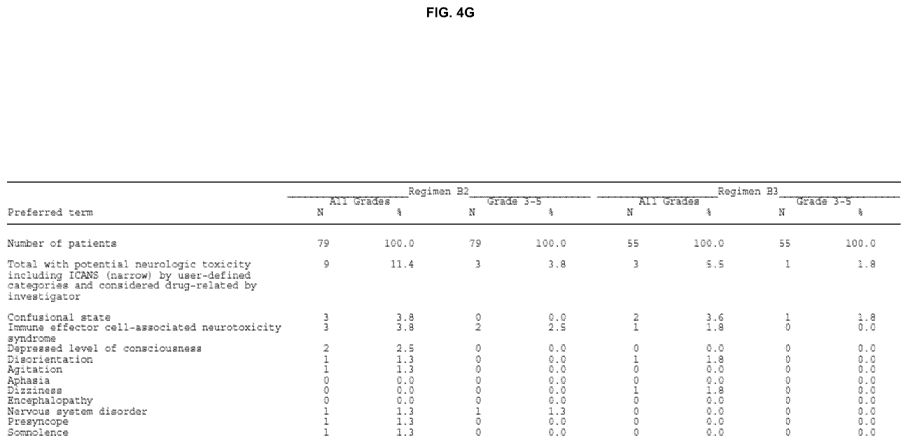

Based on reported safety per dose group it was decided to escalate the BI 764532 target dose from 90 μg/kg to 1530 μg/kg over a total of 8 dose levels (90 μg/kg, 180 μg/kg, 270 μg/kg, 360 μg/kg, 540 μg/kg, 720 μg/kg, 1080 μg/kg, 1530 μg/kg). CRS (all grades) was reported in Regimen B2 (all dose levels) in 67% of patients; most of the reported CRS events were low grade (CTCAE grade 1-2), and only approx. 3% of patients were reported with CRS CTCAE grade 3 and higher. The vast majority of the CRS events occurred at the initial BI 764532 step-in administrations, i.e. at the step-in doses of 30 μg/kg (CRS all grade 63%, CRS grade≥3 approx 1.5%) and 90 μg/kg (CRS all grade 30%, CRS grade≥3 approx 1.5%). Surprisingly, at target dose levels (after step-in) the CRS severity was low (max grade 2), CRS frequency was well controlled (max 20%) and no CRS treatment with steroids or tocilizumab was required (see FIG. 4A).

Extended step-in period—in case Grade 2 or 3 drug-related AEs occurred during step-in dosing, the investigator was allowed to continue with the same step-in dose at the next administration, or to delay the next trial drug administration. In both cases, the step-in period was extended. Thus, the target dose was reached later for individual patients. In case of additional step-in doses, additional hospitalizations were required (until target dose is reached).

Safety conclusion: while in Regimens without step-in dosing dose escalation had to be stopped at 90 μg/kg, with implementation of step-in dosing the BI 764532 target dose could be increased up to 1530 μg/kg; and even at this target dose level in Regimen B2 no MTD is defined yet.

Tumor response was assessed in all patients enrolled in Regimen B2. Best overall tumor response PR by RECIST 1.1 was reported in 16 out of 73 evaluable patients (21.9%) across all BI 764532 dose levels in Regimen B2 (see FIG. 5B).

### Example 2: Interferon-Gamma Levels at the Target Dose can be Reduced by Introduction of the Step-In Dosing

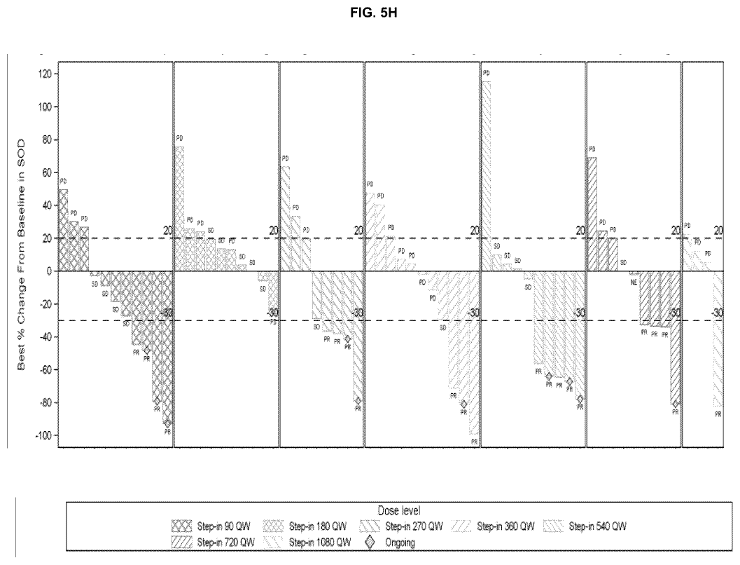

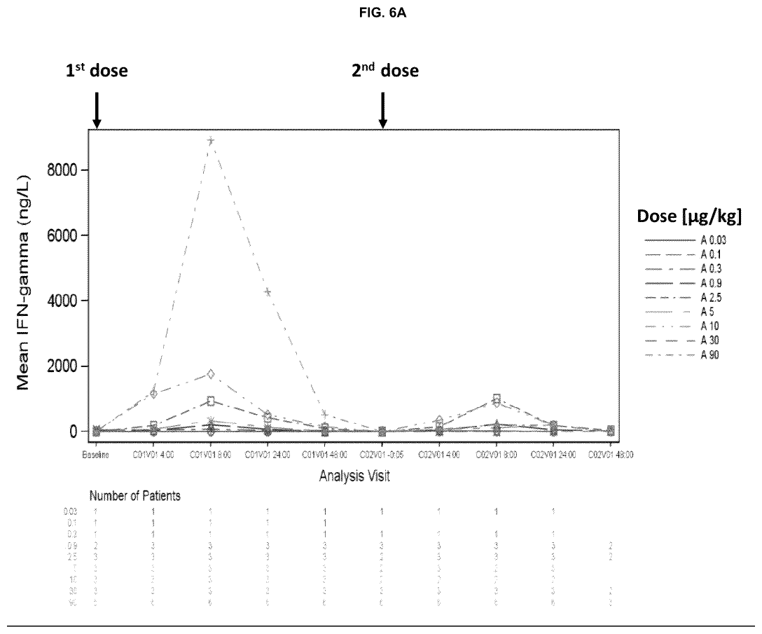

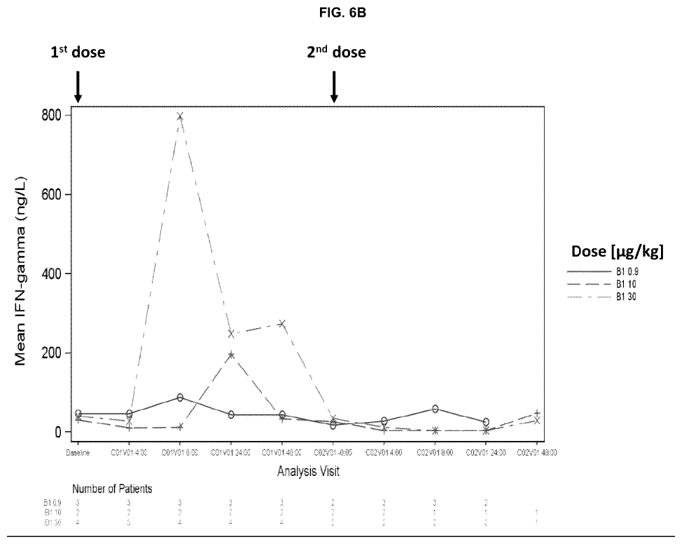

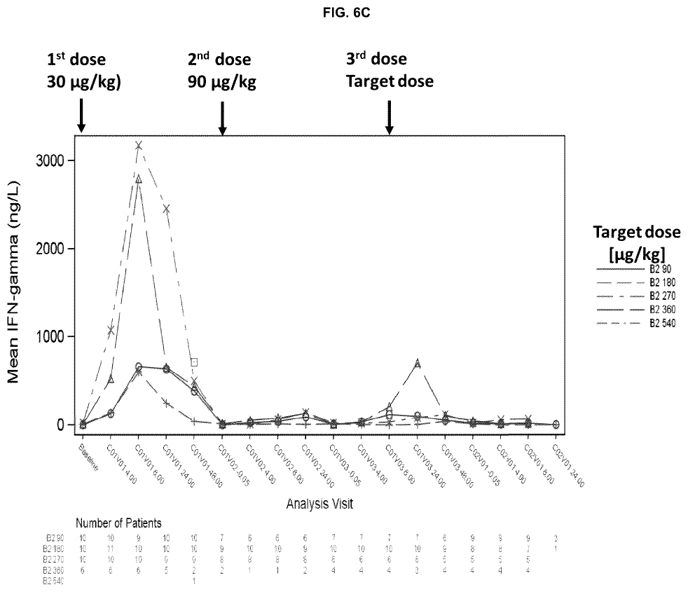

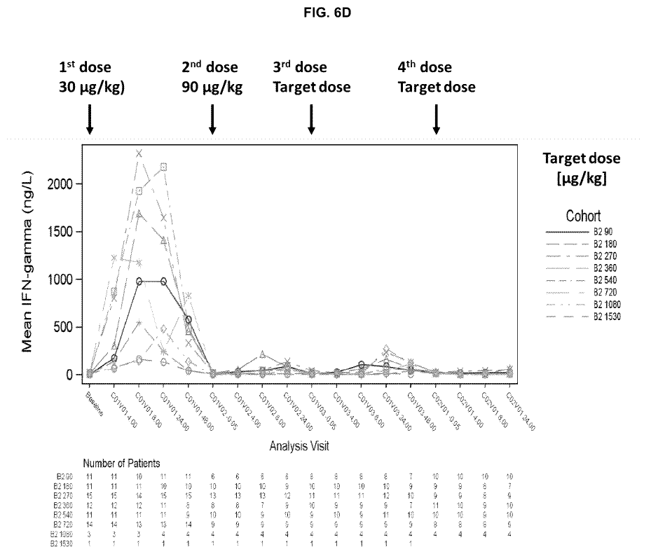

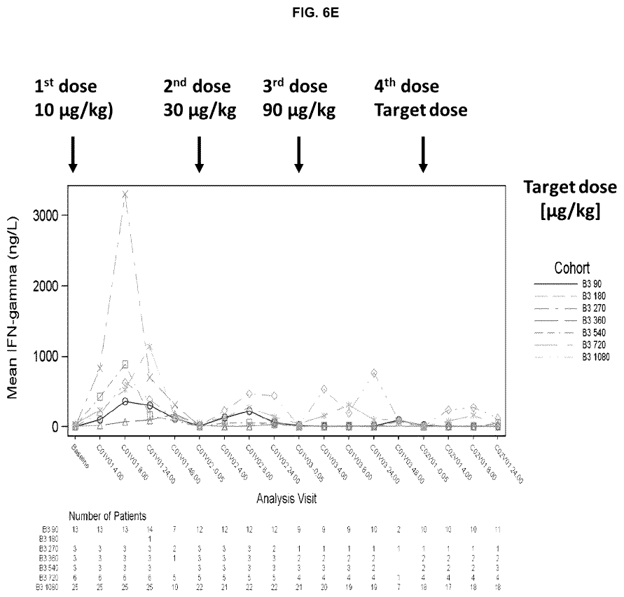

Serum levels of Interferon-gamma were quantified at various time points after administration of BI 764532. Initial peak of Interferon-gamma serum levels was observed 8 hours after administration of the first dose of BI 764532. After receiving the second dose of BI 764532 peak of Interferon-gamma levels were reduced when administered 3 weeks after the first dose (FIG. 6A, Regimen A) as well as when administered one week after the first dose (FIG. 6B, Regimen B1). When patients received a first dose of 30 μg/kg followed by a second dose of 90 μg/kg one week later, or a first dose of 10 μg/kg followed by a second dose of 30 μg/kg one week later, followed by a third dose of 90 μg/kg one week later, Interferon-gamma levels after the 3^{rd} or 4^{th} dose of 90, 180, 270, 360, 540, 720, 1080, or 1530 g/kg BI 764532 (target dose) were reduced compared to when patients received a first dose of 90 μg/kg (FIG. 6C, FIG. 6D, FIG. 6E, Regimen B2, Regimen B3).

### Example 3A: Regimen B3 Design

### Dosing Regimen B3

Treatment cycles of 3 week (21 day) duration. BI 764532 monotherapy. Dosing regimen comprising i.v. administration of a first step in dose (×1 μg/kg) in step-in cycle on day 1, a second step-in dose (×2 μg/kg) in step-in cycle on day 8, a third step-in dose (×3 μg/kg) in step-in cycle on day 15, followed by weekly i.v. administration of the target dose (x μg/kg) in treatment cycle 1 (i.e. on day 1, 8 and 15 of the 21 day cycle) followed by once every three week (q3w) administration of the target dose in repeated treatment cycles 2 (i.e. on day 1).

TABLE 4
Regimen B3:
one cycle = 3 weeks duration
Step-in cycle: initial 3 doses on weekly intervals as “step-in”
Treatment cycle 1: Target doses (90 μg/kg, 270 μg/kg,
360 μg/kg, 540 μg/kg, 720 μg/kg, or 1080 μg/kg) administered weekly
Treatment cycles 2 (repeated): Target doses (90 μg/kg, 270 μg/kg,
360 μg/kg, 540 μg/kg, 720 μg/kg, or 1080 μg/kg) administered once every
3 weeks (q3w) until progression or toxicity, maximum of 36 months

| | | | | | | | Treatment |
|---|---|---|---|---|---|---|---|
| | | | | | | | cycles 2 |
| | Step-in cycle | | | Treatment cycle 1 | | | (repeated) |
| | d 1 | d 8 | d 15 | d 1 | d 8 | d 15 | d 1 |
| dose | 10 μg/kg | 30 μg/kg | 90 μg/kg | Target dose: | Target dose: | Target dose: | Target dose: |
| | | | | 90-1080 μg/kg | 90-1080 μg/kg | 90-1080 μg/kg | from 90 to |
| | | | | | | | 1080 μg/kg |

Regimen B3 is further included in the Phase I study described in example 1 above and aims to further improve tolerability by a modified step-in as well as reduce burden of long term weekly iv administration in Regimen B2. Regime B3 is a “hybrid regimen” starting with q1w dosing and continuing in treatment cycles 2 with q3w administration.

Step-in strategy in Regimen B3: safety data for Regimen B2 (most CRS cases were observed at the step-in doses with the majority at the initial 30 μg/kg step-in dose) indicate that the initial step-in dose is critical for the overall tolerability of BI 764532 and further modifications of step-in dosing might improve the safety. With the aim to further reduce the frequency and severity of adverse events, especially CRS and potentially as well ICANS, during the step-in phase, a lower initial step-in dose of 10 μg/kg is added to the step-in regimen. Accordingly, patients in Regimen B3 will receive a first step in dose (10 μg/kg) in step-in cycle on day 1, a second step-in dose (30 μg/kg) in step-in cycle on day 8, and a third step-in dose (90 μg/kg) in step-in cycle on day 15. Administration of target dose starts one week after the 3^{rd} step-in dose, i.e. at treatment cycle 1 day 1. In case Grade 2 or 3 drug related AEs occur at step-in doses the investigator may decide to repeat at the next administration the same step-in dose or delay next drug administration to extend the step in period. For individual patients the target dose might be reached at a later time.

Target doses in Regimen B3:_based on the observation in Regimen B2 (tolerability of target doses 90-1530 μg/kg) target doses of BI 764532 will be escalated in Regimen B3 from 90 μg/kg to 1080 μg/kg over a total of 6 dose levels. In treatment cycle 1 target doses are administered weekly (i.e. treatment cycle 1 on day 1, treatment cycle 1 on day 8, and treatment cycle 1 on day 15). From treatment cycle 2 onwards target dose is administered once per cycle (cycle×day 1), i.e. once every 3 weeks (q3w) until progression or toxicity, maximum of 36 months. The rationale for “hybrid-dosing”, i.e. switching from q1w to q3w administration from treatment cycle 1 to treatment cycle 2 is that administration regimen q3w may be more convenient for patients.

Surprisingly, the overall CRS frequency (all CTCAE grades) in Regimen B3 was reduced as compared to Regimen B2 (50.9% vs 68.4%, FIGS. 4E1 and 4D1) and similarly CRS events of higher severity (CTCAE grade≥2) were reported in B3 at a lower frequency (9.1% (5.5% grade 2, 3.6% grade 3) as compared to B2 (19% (15.2% grade 2, 2.5% grade 3, 1.3% grade 4)) (FIGS. 4E1 and 4D1). Especially during the step-in cycle the reduced frequency of CRS was notable comparing Regimens B2 (CRS, all grades reported in 68.4% of patients, FIG. 4F) and B3 (CRS, all grades reported in 49.1% of patients, FIG. 4F) while the frequency of CRS was low at target dose levels (treatment cycles) and comparable in both regimens (13.6% in B2 and 16.7% in B3) (FIG. 4F). At target dose levels (after step-in) no higher grade CRS (CTCAE grade≥3) was reported and CRS treatment with steroids or tocilizumab was required only in a few cases (see FIGS. 4E and 4D).

The overall ICANS frequency (all CTCAE grades) in Regimen B3 was surprisingly reduced as compared to Regimen B2 (5.5% vs 11.4%, FIGS. 4G) and similarly ICANS events of higher severity (CTCAE grade≥3) were reported in B3 at a lower frequency (1.8%) as compared to B2 (3.8%) (FIG. 4G).

Surprisingly, the clinical efficacy of treatment as measured by best overall tumor response and disease control was similar for regimens B2 (objective response 26.6%, disease control 46.8%) and B3 (objective response 29.4%, disease control 49%) (FIG. 5C).

Conclusion: the step-in Regimen in B3 improved tolerability of BI 764532 treatment as compared to the step-in Regimen in B2, by reducing the frequency of CRS occurring at step-in doses as well as reducing the frequency of ICANS while allowing to reach similar target doses in both regimens (B2 and B3) and achieving comparable clinical efficacy.

Notably, clinical activity of BI 764532 treatment was reported in all indications (SCLC, epNEC, LCNEC) investigated in the Phase I study (FIGS. 5F, 5G)

### Example 3B (Dosing Regimens B4A, B4B, B4C, B4D)

Regimens B4a, B4b, B4c and B4d are further included in the Phase I study described in example 1 above, build on clinical results generated with Regimen B3 and aim to further optimize tolerability and time clinically active doses by a modified step-in strategy. Furthermore, Regimens B4a, B4b, B4c and B4d will start with body-weight adapted dosing with a switch to fixed doses (i.e. doses without adaptation to body weight) at subsequent administration—based on the rationale described in Example 4 below). Regimens B4a, B4b, B4c and B4d are “hybrid regimen” starting with q1w dosing and continuing in treatment cycles 2 onwards with q3w administration.

B4a: Treatment cycles of 3 week (21 day) duration. BI 764532 monotherapy. Dosing regimen comprising i.v. administration of a first step in dose (10 μg/kg) in step-in cycle on day 1, a second step-in dose (90 μg/kg) in step-in cycle on day 8, followed by administration of the body weight-independent target dose of 10 mg (Week 3). The target dose of 10 mg will further be administered weekly in treatment cycle 1 (i.e. on day 1, 8 and 15 of the 21 day cycle) followed by once every three week (q3w) administration of the target dose in repeated treatment cycles 2 (i.e. on day 1).

In case Grade 2 or 3 drug related AEs occur in step-in cycle the investigator may decide to repeat at the next administration the same step-in dose or delay next drug administration to extend the step in period (as a result for individual patients the target dose might be reached at a later time). Based on safety findings at the target dose individual target dose reduction will be allowed as needed.

Regimen B4a:
one cycle = 3 weeks duration
Step-in cycle: initial 3 doses on weekly intervals as “step-in”
Treatment cycle 1: Target dose 10 mg administered weekly
Treatment cycles 2 (repeated): Target dose 10 mg administered once every
3 weeks (q3w) until progression or toxicity, maximum of 36 months

| | | | | | | | Treatment |
|---|---|---|---|---|---|---|---|
| | | | | | | | cycles 2 |
| | Step-in cycle | | | Treatment cycle 1 | | | (repeated) |
| | d 1 | d 8 | d 15 | d 1 | d 8 | d 15 | d 1 |
| dose | 10 μg/kg | 90 μg/kg | 10 mg | Target dose: | Target dose: | Target dose: | Target dose: |
| | | | | 10 mg | 10 mg | 10 mg | 10 mg |

Regimen B4b:
one cycle = 3 weeks duration
Step-in cycle: initial 3 doses on weekly intervals as “step-in”
Treatment cycle 1: Target dose 60 mg administered weekly
Treatment cycles 2 (repeated): Target dose 60 mg administered once every
3 weeks (q3w) until progression or toxicity, maximum of 36 months

| | | | | | | | Treatment |
|---|---|---|---|---|---|---|---|
| | | | | | | | cycles 2 |
| | Step-in cycle | | | Treatment cycle 1 | | | (repeated) |
| | d 1 | d 8 | d 15 | d 1 | d 8 | d 15 | d 1 |
| dose | 10 μg/kg | 10 mg | 60 mg | Target dose: | Target dose: | Target dose: | Target dose: |
| | | | | 60 mg | 60 mg | 60 mg | 60 mg |

B4c: Treatment cycles of 3 week (21 day) duration. BI 764532 monotherapy. Dosing regimen comprising i.v. administration of a first step in dose (10 μg/kg) in step-in cycle on day 1, a second step-in dose (60 μg/kg) in step-in cycle on day 8, followed by administration of the body weight-independent target dose of 10 mg (Week 3). The target dose of 10 mg will further be administered weekly in treatment cycle 1 (i.e. on day 1, 8 and 15 of the 21 day cycle) followed by once every three week (q3w) administration of the target dose in repeated treatment cycles 2 (i.e. on day 1).

In case Grade 2 or 3 drug related AEs occur in step-in cycle the investigator may decide to repeat at the next administration the same step-in dose or delay next drug administration to extend the step in period (as a result for individual patients the target dose might be reached at a later time). Based on safety findings at the target dose individual target dose reduction will be allowed as needed.

Regimen B4c:
one cycle = 3 weeks duration
Step-in cycle: initial 3 doses on weekly intervals as “step-in”
Treatment cycle 1: Target dose 10 mg administered weekly
Treatment cycles 2 (repeated): Target dose 10 mg administered once every
3 weeks (q3w) until progression or toxicity, maximum of 36 months

| | | | | | | | Treatment |
|---|---|---|---|---|---|---|---|
| | | | | | | | cycles 2 |
| | Step-in cycle | | | Treatment cycle 1 | | | (repeated) |
| | d 1 | d 8 | d 15 | d 1 | d 8 | d 15 | d 1 |
| dose | 10 μg/kg | 60 μg/kg | 10 mg | Target dose: | Target dose: | Target dose: | Target dose: |
| | | | | 10 mg | 10 mg | 10 mg | 10 mg |

B4d: Treatment cycles of 3 week (21 day) duration. BI 764532 monotherapy. Dosing regimen comprising i.v. administration of a first step in dose (10 μg/kg) in step-in cycle on day 1, a second step-in dose (60 μg/kg) in step-in cycle on day 8, followed by administration of the body weight-independent target dose of 60 mg (Week 3). The target dose of 60 mg will further be administered weekly in treatment cycle 1 (i.e. on day 1, 8 and 15 of the 21 day cycle) followed by once every three week (q3w) administration of the target dose in repeated treatment cycles 2 (i.e. on day 1).

In case Grade 2 or 3 drug related AEs occur in step-in cycle the investigator may decide to repeat at the next administration the same step-in dose or delay next drug administration to extend the step in period (as a result for individual patients the target dose might be reached at a later time). Based on safety findings at the target dose individual target dose reduction will be allowed as needed.

Regimen B4d:
one cycle = 3 weeks duration
Step-in cycle: initial 3 doses on weekly intervals as “step-in”
Treatment cycle 1: Target dose 10 mg administered weekly
Treatment cycles 2 (repeated): Target dose 10 mg administered once every
3 weeks (q3w) until progression or toxicity, maximum of 36 months

| | | | | | | | Treatment |
|---|---|---|---|---|---|---|---|
| | | | | | | | cycles 2 |
| | Step-in cycle | | | Treatment cycle 1 | | | (repeated) |
| | d 1 | d 8 | d 15 | d 1 | d 8 | d 15 | d 1 |
| dose | 10 μg/kg | 60 μg/kg | 60 mg | Target dose: | Target dose: | Target dose: | Target dose: |
| | | | | 60 mg | 60 mg | 60 mg | 60 mg |

### Example 4. An Open-Label, Multi-Center Phase II Dose Selection Trial of Intravenous BI 764532, a DLL3-Targeting T Cell Engager, in Patients with Relapsed/Refractory Extensive-Stage Small Cell Lung Cancer and in Patients with Other Relapsed/Refractory Neuroendocrine Carcinomas

### Dosing Regimen Dareon™-5

Treatment cycles of 3 week (21 day) duration. BI 764532 monotherapy. Dosing regimen comprising a first step in dose (×1 μg/kg) in step-in cycle on day 1, a second step-in dose (×2 μg/kg) in step-in cycle on day 8, a third step-in dose (×3 μg/kg) in step-in cycle on day 15, followed by weekly administration of the target dose (fixed dose×mg) in treatment cycle 1 (i.e. on day 1, 8 and 15), followed by once every tree week (q3w) administration of the target dose in repeated treatment cycles 2 (i.e. on day 1).

Following patients are included in this study: patients with SCLC who have had progression or recurrence following at least two prior lines of therapy, including at least one platinum-based regimen and in patients with histologically or cytologically confirmed epNEC or LCNEC of the lung, who have progression or recurrence following at least one platinum-based regimen

Regimen Dareon ™-5:
1 cycle = 3 weeks duration
Step-in cycle: initial 3 doses (x μg/kg) on weekly intervals as “step-in”
Treatment cycle 1: Target doses (x mg) administered weekly
Treatment cycles 2 (repeated): Target dose (x mg) administered
once every 3 weeks (q3w) until progression or toxicity, maximum of
36 months (please, note that target doses are NOT weight based):

| | | | | | | | Treatment |
|---|---|---|---|---|---|---|---|
| | | | | | | | cycles 2 |
| | Step-in cycle | | | Treatment cycle 1 | | | (repeated) |
| | d 1 | d 8 | d 15 | d 1 | d 8 | d 15 | d 1 |
| dose | 10 μg/kg | 30 μg/kg | 90 μg/kg | Target dose: | Target dose: | Target dose: | Target dose: |
| | | | | From 5 mg | From 5 mg | From 5 mg | From 5 mg |
| | | | | to 60 mg | to 60 mg | to 60 mg | to 60 mg |

Step-in strategy in Regimen Dareon™-5: (step-in dosing with ≥3 steps and weight based dosing (10 μg/kg, 30 μg/kg, 90 μg/kg)).
Target doses in Regimen Dareon™-5: at the target doses the doses will be fixed without adaptation to body weight. The rationale for fixed target doses is the following:
1) Tolerability of a wide range of target doses in Regimen B2:
target doses between 90 μg/kg and 1530 μg/kg were tolerated in Regimen B2 without results pointing to a dose-safety correlation at the target dose (i.e. no increased AE frequency in B2 at higher target doses). This indicates that adaptions of target dose to body weight are not needed for safety reasons. Accordingly, target doses of 10 mg and 60 mg (corresponding to 125 μg/kg and 750 μg/kg for a 80 kg patient) will be tested in Regimen Dareon™-5.
2) convenience of fixed doses: fixed dosing reduces complexity of drug preparation as well as drug wasting.

In contrast to the fixed target doses applied during treatment cycles in Regimen Dareon™-5 the step-in doses in Dareon™-5 will be adapted to body weight. The rationale for weight-adapted step in dosing is as follows:
dosing/exposure is critical for tolerability at the initial administrations (during step-in)
risk of CRS is considered to correlate with body weight.
weight-based step-in dosing was determined in Regimes B2 and B3 to result in manageable safety

### Example 5. A Phase I, Non-Randomized, Open-Label, Multi-Center Dose Escalation Trial of BI 764532 Combined with Ezabenlimab in Patients with Small Cell Lung Carcinoma and Other Neuroendocrine Neoplasms Expressing DLL3

### Dosing Regimen C

Treatment cycles of 3 week (21 day) duration. BI 764532 combined with a checkpoint inhibitor (ezabenlimab).
Dosing regimen comprising a first step in BI 764532 dose (×1 μg/kg) in step-in cycleon day 1, a second step-in dose (×2 μg/kg) in step-in cycle on day 8, a third step-in dose (×3 μg/kg) in step-in cycle on day 15, followed by weekly administration of the target dose (fixed dose×mg) in treatment cycle 1 (i.e. on day 1, 8 and 15), followed by once every three week (q3w) administration of the target dose in repeated treatment cycles 2 (i.e. on day 1).
The checkpoint-inhibitor (ezabenlimab) will be administered on day 1 of each treatment cycle.

Combination of BI 764532 and ezabenlimab
1 cycle = 3 weeks duration
BI 764532 dosing (dose escalation in subsequent dose cohorts):
Step-in cycle: initial 3 doses (x μg/kg) on weekly intervals as “step-in”
Treatment cycle 1: Target doses (x mg) administered weekly
Treatment cycles 2 (repeated): Target dose (x mg) administered once every
3 weeks (q3w) until progression or toxicity, maximum of 36 months
Ezabenlimab (PD-1 inhibitor) dosing (fixed dose, no dose escalation):
240 mg i.v. infusion on day 1 in all cycles

| | | | | | | | Treatment |
|---|---|---|---|---|---|---|---|
| | | | | | | | cycles 2 |
| | Step-in cycle | | | Treatment cycle 1 | | | (repeated) |
| | d 1 | d 8 | d 15 | d 1 | d 8 | d 15 | d 1 |
| BI | 5-10 μg/ | 15-30 μg/ | 30-90 μg/ | Target dose: | Target dose: | Target dose: | Target dose: |
| 764532 | kg | kg | kg | From 5 mg to | From 5 mg to | From 5 mg to | From 5 mg to |
| dose | | | | to 60 mg | to 60 mg | to 60 mg | to 60 mg |
| Ezabenlimab | 240 mg | | | 240 mg | | | 240 mg |
| dose | | | | | | | |

Step-in strategy in Regimen C: step-in dosing will start at reduced doses compared to B2, B3, Dareon™-5: initial step-in dosing with ≥3 steps and weight-based dosing (5 μg/kg, 15 μg/kg, 30 μg/kg). If the reduced step-in in combination with ezabenlimab is tolerated well the step-in doses might be increased in subsequent dosing cohorts
Target doses in Regimen C: the fixed target doses (i.e. without adaptation to body weight) will be escalated in subsequent dosing cohorts to define the MTD/RDE.
The rationale for body weight adapted step-in doses and fixed target doses is the same as for Regimen Dareon™-5
Depending on tolerability of the combination the BI 764532 dosing days might be shifted (e.g. to avoid BI 764532 dosing on days with ezabenlimab administration).

### Example 6: Combination of BI 764532 with Topotecan in Patients with SCLC

### Dosing Regimen D

Treatment cycles of 3 week (21 day) duration. BI 764532 combined with a Topotecan.
Dosing regimen comprising a first step in BI 764532 dose (×1 μg/kg) in step-in cycle on day 1, a second step-in dose (×2 μg/kg) in step-in cycle on day 8, a third step-in dose (×3 μg/kg) in step-in cycle on day 15, followed by weekly administration of the target dose (fixed dose×mg) in treatment cycle 1 (i.e. on day 1, 8 and 15), followed by once every three week (q3w) administration of the target dose in repeated treatment cycles 2 (i.e. on day 1).
Topotecan will be administered either with a dose of 2.3 mg/m² orally (p.o.) or 1.5 mg/m² by intravenous infusion daily on Day 1 to Day 5 during each cycle every 21 days.

Combination of BI 764532 with topotecan 1 cycle = 3 weeks duration
BI 764532 dosing (dose escalation in subsequent dose cohorts):
Step-in cycle: initial 3 doses (x μg/kg) on weekly intervals as “step-in”
Treatment cycle 1: Target doses (x mg) administered weekly
Treatment cycles 2: Target dose (x mg) administered once every
3 weeks (q3w) until progression or toxicity, maximum of 36 months
Topotecan: 2.3 mg/m² orally (p.o.) OR 1.5 mg/m² by intravenous
infusion daily on Day 1 to Day 5 during each cycle every 21 days.
If topotecan treatment is stopped (e.g. because planned
treatment duration is reached or for tolerability reasons)
BI 764532 treatment will continue as monotherapy

| | | | | | | | Treatment |
|---|---|---|---|---|---|---|---|
| | | | | | | | cycle 2 |
| | Step-in cycle | | | Treatment cycle 1 | | | (repeated) |
| | d 1 | d 8 | d 15 | d 1 | d 8 | d 15 | d 1 |
| BI | 10 μg /kg | 30 μg /kg | 90 μg /kg | Target dose: | Target dose: | Target dose: | Target dose: |
| 764532 | | | | From 5 mg | From 5 mg | From 5 mg | From 5 mg |
| dose | | | | to 60 mg | to 60 mg | to 60 mg | to 60 mg |
| | d 1-5 | | | d 1-5 | | | d 1-5 |
| Topotecan | 2.3 mg/m²/d p.o. | | | 2.3 mg/m²/d p.o. | | | 2.3 mg/m²/d p.o. |
| | OR | | | OR | | | OR |
| | 1.5 mg/m²/d i.v. | | | 1.5 mg/m²/d i.v. | | | 1.5 mg/m²/d i.v. |

Step-in strategy in Regimen D: step-in dosing will apply weight based dosing based on Regimen B3 results
Target doses in Regimen D: the fixed target doses (i.e. without adaptation to body weight) will be escalated in subsequent dosing cohorts (e.g. 10 mg, 30 mg, 60 mg) to define the MTD/RDE.
The rationale for body weight adapted step-in doses and fixed target doses is the same as for Regimen Dareon™-5

### Example 7: Combination of BI 764532 with Platin+Etoposide (SOC for 1 L EPNEC, LC NEC Lung, NEC of Unknown Primary Site) in Patients with EPNEC, Pulmonary LCNEC and NEC of Unknown Primary Site

### Dosing Regimen E

Treatment cycles of 3 week (21 day) duration. BI 764532 combined with a platin+eptoposide.
Treatment with BI 764532 will start after an initial cycle of platin+etoposide is completed (i.e. the step-in cycle of the combination BI 764532+platin+etoposide (described as step-in cycle below) is the 2^{nd} cycle of platinum+etoposide)
BI 764532 dosing regimen comprising a first step-in dose (×1 μg/kg) in step-in cyle on day 1, a second step-in dose (×2 μg/kg) in step-in cycle on day 8, a third step-in dose (×3 μg/kg) in step-in cycle on day 15, followed by weekly administration of the target dose (fixed dose×mg) in combination treatment cycle 1 (i.e. on day 1, 8 and 15), followed by once every three week (q3w) administration of the target dose in treatment cycles 2 (i.e. on day 1).
Platin will be administered on Day 1 of each cycle, etoposide will be administered on days 1-3 of each cycle.
A total of 3-5 cycles of BI 764532+platin+etoposide will be administered. After completion of BI 764532+platin+etoposide, monotherapy with BI 764532 will continue until progression or toxicity for a maximum of 36 months

Combination of BI 764532 with platin + etoposide (SoC for 1 L epNEC, LC NEC lung, NEC of unknown primary site)
1 cycle = 3 weeks duration
BI 764532 + platin + etoposide will start after an initial cycle of platin + eptoposide
is completed (i.e. the step-in cycle of the combination BI 764532 + platin + etoposide
(described as step-in cycle below) is the 2nd cycle of platinum + etoposide)
BI 764532 dosing (dose escalation in subsequent dose cohorts):
Step-in cycle: initial 3 doses (x μg/kg) on weekly intervals as “step-in”
Treatment cycle 1: Target doses (x mg) administered weekly
Treatment cycle 2 (repeated): Target dose (x mg) administered once every 3 weeks (q3w) until progression or toxicity, maximum of 36 months
Platin: d 1 of each cycle
Etoposide: d 1-3 of each cycle

| | | | | | | | Treatment | |
|---|---|---|---|---|---|---|---|---|
| | | | | | | | cycles 2 | |
| | | | | | | | (combi) | Treatment |
| | | | | | | | administered 1 | cycles 2 |
| | Step-in cycle | | | Treatment cycle 1 | | | up to 3 times | (mono) |
| | d 1 | d 8 | d 15 | d 1 | d 8 | d 15 | d 1 | D 1 |
| BI | 10 μg/ | 30 μg/ | 90 μg/ | Target dose: | Target dose: | Target dose: | Target dose: | Target dose: |
| 764532 | kg | kg | kg | From 5 mg | From 5 mg | From 5 mg | From 5 mg | From 5 mg |
| dose | | | | to 60 mg | to 60 mg | to 60 mg | to 60 mg | to 60 mg |
| Platin | Carboplatin | | | Carboplatin | | | Carboplatin | |
| | AUC 5 mg/mL min | | | AUC 5 mg/mL min | | | AUC 5 mg/mL min | |
| | OR Cisplatin | | | OR Cisplatin | | | OR Cisplatin | |
| | 75 mg/m2 | | | 75 mg/m2 | | | 75 mg/m2 | |
| | d 1-3 | | | d 1-3 | | | d 1-3 | |
| Etopopside | 100 mg/m2 | | | 100 mg/m2 | | | 100 mg/m2 | |

Step-in strategy in Regimen E: step-in dosing will apply weight-based dosing based on Regimen B3 results
Target doses in Regimen E: the fixed target doses (i.e. without adaptation to body weight) will be escalated in subsequent dosing cohorts (e.g. 10 mg, 30 mg, 60 mg) to define the MTD/RDE.
The rationale for body weight adapted step-in doses and fixed target doses is the same as for Regimen Dareon™-5

### Example 8: Combination of BI 764532 with Platin+Etoposide+Anti-Pd-L1 (SOC for 1 L SCLC) in Patients with Extensive-Stage Small Cell Lung Carcinoma

### Dosing Regimen F

Treatment cycles of 3 week (21 day) duration. BI 764532 combined with a platin+eptoposide+aPD-L1 (e.g. atezolizumab, durvalumab).
Treatment with BI 764532 will start after an initial cycle of platin+etoposide+aPD-L1 is completed (i.e. the step-in cycle of the combination BI 764532+platin+etoposide+aPD-L1 (described as step-in cycle below) is the 2^{nd} cycle of platinum+etoposide+aPD-L1)
BI 764532 dosing regimen comprising a first step-in dose (×1 μg/kg) in step-in cycle on day 1, a second step-in dose (×2 μg/kg) in step-in cycle on day 8, a third step-in dose (×3 μg/kg) in step-in cycle on day 15, followed by weekly administration of the target dose (fixed dose×mg) in treatment cycle 1 (i.e. on day 1, 8 and 15), followed by once every three week (q3w) administration of the target dose in repeated treatment cycles 2 (i.e. on day 1).
Platin will be administered on Day 1 of each cycle, etoposide will be administered on days 1-3 of each cycle, aPD-L1 will be administered on day 1 of each cycle.
A total of 3-5 cycles of BI 764532+platin+etoposide+aPD-L1 will be administered. After completion of BI 764532+platin+etoposide+aPD-L1, maintenance treatment (aPD-L1+BI 764532 or monotherapy with BI 764532) will continue until progression or toxicity for a maximum of 36 months.

Combination of BI 764532 with platin + etoposide + anti-PD-L1 (SoC for 1 L SCLC)
1 cycle = 3 weeks duration
BI 764532 + platin + etoposide + aPD-L1 will start after an initial cycle of platin + etoposide + aPD-L1
is completed (i.e. the first cycle of the combination BI 764532 + platin + etoposide +
aPD-L1 (described as step-in cycle below) is the 2nd cycle of platinum + etoposide + aPD-L1)
BI 764532 dosing (dose escalation in subsequent dose cohorts):
Step-in cycle: initial 3 doses (x μg/kg) on weekly intervals as “step-in”
Treatment cycle 1: Target doses (x mg) administered weekly
Treatment cycles 2 (repeated): Target dose (x mg) administered once every 3 weeks (q3w) until progression or toxicity, maximum of 36 months
Platin: d 1 of each cycle
Etoposide: d 1-3 of each cycle

| | | | | | | | Treatment | Treatment |
|---|---|---|---|---|---|---|---|---|
| | | | | | | | cycles 2 | cycles 2 |
| | Step-in cycle | | | Treatment cycle 1 | | | (combi) | (mono) |
| | d 1 | d 8 | d 15 | d 1 | d 8 | d 15 | d 1 | d 1 |
| BI | 10 μg/ | 30 μg/ | 90 μg/ | Target dose: | Target dose: | Target dose: | Target dose: | Target dose: |
| 764532 | kg | kg | kg | From 5 mg | From 5 mg | From 5 mg | From 5 mg | From 5 mg |
| dose | | | | to 60 mg | to 60 mg | to 60 mg | to 60 mg | to 60 mg |
| SoC | Carboplatin | | | Carboplatin | | | Carboplatin | |
| | AUC 5 mg/mL min | | | AUC 5 mg/mL min | | | AUC 5 mg/mL min | |
| | OR Cisplatin | | | OR Cisplatin | | | OR Cisplatin | |
| | 75 mg/m2 | | | 75 mg/m2 | | | 75 mg/m2 | |
| aPD-L1 | Atezolizumab | | | Atezolizumab | | | Atezolizumab | Atezolizumab |
| | OR Durvalumab | | | OR Durvalumab | | | OR Durvalumab | OR Durvalumab |
| | | | | | | | | (will be |
| | | | | | | | | dose q4w) |
| | d 1-3 | | | d 1-3 | | | d 1-3 | |
| Etopopside | 100 mg/m2 | | | 100 mg/m2 | | | 100 mg/m2 | |

Step-in strategy in Regimen F: step-in dosing will apply weight based dosing based on Regimen B3 results
Target doses in Regimen F: the fixed target doses (i.e. without adaptation to body weight) will be escalated in subsequent dosing cohorts (e.g. 10 mg, 30 mg, 60 mg) to define the MTD/RDE.
The rationale for body weight adapted step-in doses and fixed target doses is the same as for Regimen Dareon™-5

### Example 9: Phase I Study of BI 764532 in Glioma

The study described herein is a first-in-human, Phase I dose escalation study of BI 764532 monotherapy in patients with glioma expressing DLL3 (NCT05916313).

The objectives of this study (Part A) are to characterize the dose-tolerability curve for BI 764532 in patients with diffuse glioma expressing DLL3 by assessing multiple increasing doses. The main objectives are to characterise safety of BI 764532 by assessing multiple doses with overdose control and to determine the maximum tolerated dose (MTD) and the recommended dose for expansion (RDE) in patients with diffuse glioma expressing DLL3. The main objective of this study (Part B—Expansion) is to confirm safety at the RDE.

### Methods and Analysis

### Study Design

This Phase I study is a multicenter, open-label, non-randomized, dose-escalation study. In the Part A BI 764532 is administered multiple increasing doses. In Part B (Expansion) BI 764532 is administered at dose(s) determined RDE in Part A to confirm safety

### Dosing Regimen

Intravenous (i.v.) administration. BI 764532 will be administered in a step-in dosing regimen. That is, the initial 3 administrations will be composed of a fraction of the target dose. Thereafter, the full dose (=target dose) will be administered weekly for 3 weeks, and then every 3 weeks.

Glioma Ph1 dosing regimen:
one cycle = 3 weeks duration
Step-in cycle: initial 3 doses on weekly intervals as “step-in”
Treatment cycle 1: provisional target doses (90 μg/kg,
180 μg/kg, 360 μg/kg, or 720 μg/kg) administered weekly
Treatment cycles 2 (repeated): provisional target doses 90 μg/kg,
180 μg/kg, 360 μg/kg, or 720 μg/kg) administered once every 3
weeks (q3w) until progression or toxicity, maximum of 36 months.
Further doses (step-in and target doses; doses between and above the
depicted dose levels) may be investigated based on safety results

| | | | | | | | Treatment |
|---|---|---|---|---|---|---|---|
| | | | | | | | cycles 2 |
| | Step-in cycle | | | Treatment cycle 1 | | | (repeated) |
| | d 1 | d 8 | d 15 | d 1 | d 8 | d 15 | d 1 |
| dose | 10 μg/kg | 10-30 μg/kg | 30-90 μg/kg | Target dose: | Target dose: | Target dose: | Target dose: |
| | | | | 90-720 μg/kg | 90-720 μg/kg | 90-720 μg/kg | 90-720 μg/kg |

### Main Inclusion Criteria

Patients with histologically confirmed primary progressive diffuse glioma who have failed standard of care therapies.
Eligible tumour histologies include:
   astrocytoma IDH mutant, CNS WHO Grade 2-4
   oligodendroglioma IDH mutant and 1p19q co-deleted, CNS WHO Grade 2 and 3
   glioblastoma (IDH wild type) according to the WHO 2021 brain tumour classification
Tumours must be positive for DLL3 expression
   STUDY endpoints

### Part A—Dose Escalation:

Primary endpoint: Occurrence of dose-limiting toxicity (DLT) during the MTD evaluation period
Secondary endpoint: Occurrence of DLT during the entire treatment period

### Part B—Expansion:

Primary endpoint: Occurrence of DLT during the entire treatment period
Secondary endpoints: None
   Statistical analysis, Determination of the MTD

Dose escalation is guided by Bayesian logistic regression model (BLRM) with overdose control that will be fitted to binary toxicity outcomes. The estimate of parameters will be updated as data are accumulated using the BLRM. At the end of the dose escalation, the toxicity probability at each dose level will be calculated to determine an estimate of the MTD and/or the RDE. Descriptive analysis (summary statistics) will be used to describe other safety and efficacy endpoints.

1. A T-cell engaging protein comprising a first antigen binding unit that specifically binds to DLL3 and a second antigen binding unit that specifically binds to CD3 for use in a method of treatment of a DLL3-positive cancer or a cancer selected from a Neuroendocrine Neoplasm (NEN), Merkel cell carcinoma (MCC), medullary thyroidal carcinoma and glioma,
the method comprising a step-in cycle, said step-in cycle comprising the steps of:
administering to a subject an initial dose of said T-cell engaging protein, said initial dose ranging from about 5 μg/kg of the subject's body weight to about 40 μg/kg of the subject's body weight,
administering to the subject an elevated dose, which is elevated as compared to said initial dose, of said T-cell engaging protein, the elevated dose ranging from about 15 μg/kg of the subject's body weight to about 100 μg/kg of the subject's body weight, or the elevated dose ranging from about 5 mg to about 20 mg, and
administering to the subject a further dose of the T-cell engaging protein, said further dose ranging from about 80 μg/kg of the subject's body weight to about 1600 μg/kg of the subject's bodyweight, wherein said further dose is equal to or higher than said elevated dose, or said further dose is ranging from about 5 mg to about 70 mg.

2. A T-cell engaging protein comprising a first antigen binding unit that specifically binds to DLL3 and a second antigen binding unit that specifically binds to CD3 for use in a method of treatment of a DLL3-positive cancer or a cancer selected from a Neuroendocrine Neoplasm (NEN), Merkel cell carcinoma (MCC), medullary thyroidal carcinoma and glioma,
the method comprising a step-in cycle, said step-in cycle comprising the steps of:
administering to a subject an initial dose of said T-cell engaging protein, said initial dose ranging from about 5 μg/kg of the subject's body weight to about 15 μg/kg of the subject's body weight,
administering to the subject an elevated dose, which is elevated as compared to said initial dose, of said T-cell engaging protein, the elevated dose ranging from about 15 μg/kg of the subject's body weight to about 100 μg/kg of the subject's body weight, or the elevated dose ranging from about 5 mg to about 20 mg, and
administering to the subject a further dose of the T-cell engaging protein, said further dose ranging from about 5 mg to about 70 mg.

3. A T-cell engaging protein comprising a first antigen binding unit that specifically binds to DLL3 and a second antigen binding unit that specifically binds to CD3 for use in a method of treatment of a DLL3-positive cancer or a cancer selected from a Neuroendocrine Neoplasm (NEN), Merkel cell carcinoma (MCC), medullary thyroidal carcinoma and glioma,
the method comprising a step-in cycle, said step-in cycle comprising the steps of:
administering to a subject an initial dose of said T-cell engaging protein, said initial dose ranging from about 20 μg/kg of the subject's body weight to about 40 μg/kg of the subject's body weight,
administering to the subject an elevated dose, which is elevated as compared to said initial dose, of said T-cell engaging protein, the elevated dose ranging from about 60 μg/kg of the subject's body weight to about 100 μg/kg of the subject's body weight,
administering to the subject a further dose of the T-cell engaging protein, said further dose ranging from about 80 μg/kg of the subject's body weight to about 1600 μg/kg of the subject's bodyweight, wherein said further dose is equal to or higher than said elevated dose.

4. A T-cell engaging protein comprising a first antigen binding unit that specifically binds to DLL3 and a second antigen binding unit that specifically binds to CD3 for use in a method of treatment of a DLL3-positive cancer or a cancer selected from a Neuroendocrine Neoplasm (NEN), Merkel cell carcinoma (MCC), medullary thyroidal carcinoma and glioma, the method comprising a step-in cycle, the step-in cycle comprising the steps of:
administering to the subject an initial dose, an elevated dose, which is elevated as compared to said initial dose, and a further dose of said T-cell engaging protein, wherein a ratio between said initial, elevated and further dose per kilogram of the subject's body weight is about 1: about 3: about 9.

5. A T-cell engaging protein comprising a first antigen binding unit that specifically binds to DLL3 and a second antigen binding unit that specifically binds to CD3 for use in a method of treatment of a DLL3-positive cancer or a cancer selected from a Neuroendocrine Neoplasm (NEN), Merkel cell carcinoma (MCC), medullary thyroidal carcinoma and glioma,
the method comprising a step-in cycle, the step-in cycle comprising the steps of:
administering to a subject an initial dose of said T-cell engaging protein, said initial dose ranging from about 5 μg/kg of the subject's body weight to about 15 μg/kg of the subject's body weight,
administering to the subject an elevated dose, which is elevated as compared to said initial dose, of said T-cell engaging protein, the elevated dose ranging from about 20 μg/kg of the subject's body weight to about 40 μg/kg of the subject's body weight,
administering to the subject a further dose of said T-cell engaging protein, said further dose ranging from about 80 μg/kg of the subject's body weight to about 1600 μg/kg of the subject's bodyweight.

6. The T-cell engaging protein for use according to claim 1, wherein
said initial dose, said elevated dose and/or said further is/are administered once; or
said initial dose, said elevated dose and/or said further is/are administered more than once, optionally 2 to 10 times,
wherein optionally the number of administrations is determined based on treatment tolerability including presence and/or degree of a cytokine release syndrome (CRS) and/or of an immune effector cell-associated neurotoxicity syndrome (ICANS),
and wherein further optionally said elevated dose follows the initial dose only if said CRS, to the extent determined, is rated grade 1 or less in terms of NCI CTC grade and/or if said ICANS, to the extend determined, is rated grade 1 or less, and/or wherein further optionally said further dose follows the elevated dose only if said CRS, to the extent determined, is rated grade 1 or less in terms of NCI CTC grade and/or if said ICANS, to the extend determined, is rated grade 1 or less.

7. The T-cell engaging protein for use according to claim 1; the method further comprising a first treatment cycle following the step-in cycle, said first treatment cycle comprising the steps of:
administering to a subject a first target dose of said T-cell engaging protein, said first target dose ranging from about 80 μg/kg of the subject's body weight to about 1600 μg/kg of the subject's body weight,
administering to the subject a second target dose of said T-cell engaging protein, said second dose ranging from about 80 μg/kg of the subject's body weight to about 1600 μg/kg of the subject's body weight, and
administering to the subject a third target dose of said T-cell engaging protein, said third dose ranging from about 80 μg/kg of the subject's body weight to about 1600 μg/kg of the subject's bodyweight,
wherein optionally the amount of all three target doses is identical.

8. The T-cell engaging protein for use according to claim 1, the method further comprising a first treatment cycle following said step-in cycle, said first treatment cycle comprising the steps of:
administering to a subject a first target dose of the T-cell engaging protein, said first target dose ranging from about 5 mg to about 70 mg,
administering to the subject a second dose of the T-cell engaging protein, said second dose ranging from about 5 mg to about 70 mg, and
administering to the subject a third dose of the T-cell engaging protein, said third dose ranging from about 5 mg to about 70 mg,
wherein optionally the amount of all three target doses is identical.

9. The T-cell engaging protein for use according to claim 7, wherein said first treatment cycle is followed by a second treatment cycle, said second treatment cycle comprising the step of or consisting of the step:
administering to the subject a target dose of said T-cell engaging protein, said target dose ranging from about 80 μg/kg of the subject's body weight to about 1600 μg/kg of the subject's body weight;
wherein said second treatment cycle is optionally repeated at least once.

10. The T-cell engaging protein for use according to claim 7, wherein said the first treatment cycle is followed by a second treatment cycle, said second treatment cycle comprising the step of or consisting of the step:
administering to the subject a target dose of the T-cell engaging protein, said target dose ranging from about 5 mg to about 70 mg;
wherein said second treatment cycle is optionally repeated at least once.

11. The T-cell engaging protein for use according to claim 7, wherein said first treatment cycle directly follows said step-in cycle; and/or said second treatment cycle directly follows said first treatment cycle.

12. The T-cell engaging protein for use according to claim 1, wherein said Neuroendocrine Neoplasm (NEN) is a Neuroendocrine Carcinoma (NEC), preferably selected from the group consisting of Lung-NEC such as LCNEC of the lung, Extrapulmonary (ep) NEC including NEC with unknown primary site, small cell lung cancer (SCLC) including extensive-stage (ES)-SCLC, and limited-stage (LS) SCLC; or said NEN is a Neuroendocrine Tumor (NET) such as grade 3 NET.

13. The T-cell engaging protein for use according to claim 1, wherein said T-cell engaging protein comprises a first antigen binding unit specifically binding to DLL3, comprising light chain CDRs comprising the amino acid sequences of SEQ ID NO: 13 (CDR1), SEQ ID NO: 14 (CDR2) and SEQ ID NO:15 (CDR3) and heavy chain CDRs comprising the amino acid sequences of SEQ ID NO: 16 (CDR1), SEQ ID NO: 17 (CDR2) and SEQ ID NO:18 (CDR3) and a second antigen binding unit specifically binding to CD3 comprising light chain CDRs comprising the amino acid sequences of SEQ ID NO:55 (CDR1), SEQ ID NO:56 (CDR2) and SEQ ID NO:57 (CDR3) and heavy chain CDRs comprising the amino acid sequences of SEQ ID NO:58 (CDR1), SEQ ID NO:59 (CDR2) and SEQ ID NO:60 (CDR3).

14. The T-cell engaging protein for use according to claim 1, wherein at least one further anti-cancer agent is to be administered, said further anti-cancer agent preferably being a chemotherapeutic agent and/or an immune-checkpoint inhibitor such as an anti-PD-1 antibody or an anti-PD-L1 antibody.

15. The T-cell engaging protein for use according to claim 4, to the extent they refer to claims 4 and 5, wherein said initial dose is about 10 μg/kg of the subject's body weight;
said elevated dose is about 30 μg/kg of the subject's body weight; and
said further dose is about is 90 μg/kg of the subject's body weight.


SEQUENCE LISTING (90 sequences)

SEQ ID NO: 1 - protein, 12 aa, synthetic construct
RASQSVSSNFLV

SEQ ID NO: 2 - protein, 7 aa, synthetic construct
GASTRAS

SEQ ID NO: 3 - protein, 9 aa, synthetic construct
QQYGDSPYT

SEQ ID NO: 4 - protein, 10 aa, synthetic construct
GNTFTNYYMH

SEQ ID NO: 5 - protein, 17 aa, synthetic construct
IIDPSVGSKSYAQKFLG

SEQ ID NO: 6 - protein, 12 aa, synthetic construct
AGKRFGESYFDY

SEQ ID NO: 7 - protein, 11 aa, synthetic construct
RASQGISNYLA

SEQ ID NO: 8 - protein, 7 aa, synthetic construct
AASSLQS

SEQ ID NO: 9 - protein, 9 aa, synthetic construct
LQHNSSPYT

SEQ ID NO: 10 - protein, 10 aa, synthetic construct
GYTFTSYYMH

SEQ ID NO: 11 - protein, 17 aa, synthetic construct
IINPSGGSTSYAQKFQG

SEQ ID NO: 12 - protein, 15 aa, synthetic construct
GEAVGGNYYYYGMDV

SEQ ID NO: 13 - protein, 11 aa, synthetic construct
RASQGISNYLV

SEQ ID NO: 14 - protein, 7 aa, synthetic construct
AVSSLYS

SEQ ID NO: 15 - protein, 9 aa, synthetic construct
LQHDSYPYT

SEQ ID NO: 16 - protein, 10 aa, synthetic construct
GYTFTSYYVH

SEQ ID NO: 17 - protein, 17 aa, synthetic construct
IINPGGGTTSYAQKFLG

SEQ ID NO: 18 - protein, 15 aa, synthetic construct
GEAVTGNYFYYGMDV

SEQ ID NO: 19-36 - skipped (18 entries)

SEQ ID NO: 37 - protein, 108 aa, synthetic construct
EIVLTQSPGTLSLSPGERATLSCRASQSVSSNFLVWYQQKPGQAPRPLIYGASTRASGIPDRFSGSGSGADFTLTISRLEPEDFALYYCQQYGDSPYTFGQGTTLEIK

SEQ ID NO: 38 - protein, 121 aa, synthetic construct
QVQLVQSGAEVKKPGASVKVSCKASGNTFTNYYMHWVRQAPGPGLEWMGIIDPSVGSKSYAQKFLGRVTIARDTSTSTVFLDLYSLRSEDTAVYFCARAGKRFGESYFDYWGQGTLVTVSS

SEQ ID NO: 39 - protein, 107 aa, synthetic construct
DIQMTQSPSAMSASVGDRVTITCRASQGISNYLAWFQQKPGKVPEPLIYAASSLQSGVPSRFSGSGSVTEFTLTISSLQPEDFATYYCLQHNSSPYTFGQGTKLEIK

SEQ ID NO: 40 - protein, 124 aa, synthetic construct
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGEAVGGNYYYYGMDVWGQGTTVTVSS

SEQ ID NO: 41 - protein, 107 aa, synthetic construct
DIQMTQSPSAMSASVGDRVTITCRASQGISNYLVWFQQKPGKAPKRLIYAVSSLYSGVPSRFSGSGSGTEFTLTISSLQPEDFATYYCLQHDSYPYTFGQGTKLEIK

SEQ ID NO: 42 - protein, 124 aa, synthetic construct
QVQLVQSGAEVKKPGASVKVSCKASGYTFTSYYVHWVRQAPGQGLEWMVIINPGGGTTSYAQKFLGRVTMTRDTSTNTVYMELKSLRSEDTAVYYCARGEAVTGNYFYYGMDVWGQGTTVTVSS

SEQ ID NO: 43-48 - skipped (6 entries)

SEQ ID NO: 49 - protein, 477 aa, synthetic construct
EIVLTQSPGTLSLSPGERATLSCRASQSVSSNFLVWYQQKPGQAPRPLIYGASTRASGIPDRFSGSGSGADFTLTISRLEPEDFALYYCQQYGDSPYTFGQGTTLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSEGKSSGSGSESKSTEGKSSGSGSESKSTGGGGSQVQLVQSGAEVKKPGASVKVSCKASGNTFTNYYMHWVRQAPGPGLEWMGIIDPSVGSKSYAQKFLGRVTIARDTSTSTVFLDLYSLRSEDTAVYFCARAGKRFGESYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 50 - protein, 479 aa, synthetic construct
DIQMTQSPSAMSASVGDRVTITCRASQGISNYLAWFQQKPGKVPEPLIYAASSLQSGVPSRFSGSGSVTEFTLTISSLQPEDFATYYCLQHNSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSEGKSSGSGSESKSTEGKSSGSGSESKSTGGGGSQVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGEAVGGNYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 51 - protein, 479 aa, synthetic construct
DIQMTQSPSAMSASVGDRVTITCRASQGISNYLVWFQQKPGKAPKRLIYAVSSLYSGVPSRFSGSGSGTEFTLTISSLQPEDFATYYCLQHDSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSEGKSSGSGSESKSTEGKSSGSGSESKSTGGGGSQVQLVQSGAEVKKPGASVKVSCKASGYTFTSYYVHWVRQAPGQGLEWMVIINPGGGTTSYAQKFLGRVTMTRDTSTNTVYMELKSLRSEDTAVYYCARGEAVTGNYFYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 52-54 - skipped (3 entries)

SEQ ID NO: 55 - protein, 14 aa, synthetic construct
RSSTGAVTTSNYAN

SEQ ID NO: 56 - protein, 7 aa, synthetic construct
GTNKRAP

SEQ ID NO: 57 - protein, 9 aa, synthetic construct
ALWYSNLWV

SEQ ID NO: 58 - protein, 10 aa, synthetic construct
GFTFNTYAMN

SEQ ID NO: 59 - protein, 19 aa, synthetic construct
RIRSKYNNYATYYADSVKD

SEQ ID NO: 60 - protein, 14 aa, synthetic construct
HGNFGNSYVSWFAY

SEQ ID NO: 61 - protein, 14 aa, synthetic construct
RSSTGAVTTSNYAN

SEQ ID NO: 62 - protein, 7 aa, synthetic construct
GTNKRAP

SEQ ID NO: 63 - protein, 9 aa, synthetic construct
ALWYSNLWV

SEQ ID NO: 64 - protein, 10 aa, synthetic construct
GFTFNTYAMN

SEQ ID NO: 65 - protein, 19 aa, synthetic construct
RIRSKYINYATYYADSVKD

SEQ ID NO: 66 - protein, 14 aa, synthetic construct
HGNFGNSYVSWFAY

SEQ ID NO: 67 - protein, 109 aa, synthetic construct
EAVVTQEPSLTVSPGGTVTLTCRSSTGAVTTSNYANWVQEKPGQLPRGLIGGTNKRAPWVPARFSGSLLGGKAALTLSGAQPEDEAEYFCALWYSNLWVFGGGTKLTVL

SEQ ID NO: 68 - protein, 125 aa, synthetic construct
EVQLVESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSA

SEQ ID NO: 69 - protein, 109 aa, synthetic construct; identical to SEQ ID NO: 67

SEQ ID NO: 70 - protein, 125 aa, synthetic construct
EVQLVESGGGLVQPGGSLKLSCAASGFTFNTYAMNWVRQAPGKGLEWVARIRSKYINYATYYADSVKDRFTISRDDSKNTAYLQMNNLKTEDTAVYYCVRHGNFGNSYVSWFAYWGQGTLVTVSA

SEQ ID NO: 71 - protein, 481 aa, synthetic construct
EAVVTQEPSLTVSPGGTVTLTCRSSTGAVTTSNYANWVQEKPGQLPRGLIGGTNKRAPWVPARFSGSLLGGKAALTLSGAQPEDEAEYFCALWYSNLWVFGGGTKLTVLGQPKAAPSVTLFPPSSEELQANKATLVCLISDFYPGAVKVAWKADGSPVNTGVETTTPSKQSNNKYAASSYLSLTPEQWKSHRSYSCQVTHEGSTVEKTVAPAECSGGGGSEGKSSGSGSESKSTEGKSSGSGSESKSTGGGGSEVQLVESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 72 - protein, 481 aa, synthetic construct
EAVVTQEPSLTVSPGGTVTLTCRSSTGAVTTSNYANWVQEKPGQLPRGLIGGTNKRAPWVPARFSGSLLGGKAALTLSGAQPEDEAEYFCALWYSNLWVFGGGTKLTVLGQPKAAPSVTLFPPSSEELQANKATLVCLISDFYPGAVKVAWKADGSPVNTGVETTTPSKQSNNKYAASSYLSLTPEQWKSHRSYSCQVTHEGSTVEKTVAPAECSGGGGSEGKSSGSGSESKSTEGKSSGSGSESKSTGGGGSEVQLVESGGGLVQPGGSLKLSCAASGFTFNTYAMNWVRQAPGKGLEWVARIRSKYINYATYYADSVKDRFTISRDDSKNTAYLQMNNLKTEDTAVYYCVRHGNFGNSYVSWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 73 - protein, 703 aa, synthetic construct
EIVLTQSPGTLSLSPGERATLSCRASQSVSSNFLVWYQQKPGQAPRPLIYGASTRASGIPDRFSGSGSGADFTLTISRLEPEDFALYYCQQYGDSPYTFGQGTTLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSEGKSSGSGSESKSTEGKSSGSGSESKSTGGGGSQVQLVQSGAEVKKPGASVKVSCKASGNTFTNYYMHWVRQAPGPGLEWMGIIDPSVGSKSYAQKFLGRVTIARDTSTSTVFLDLYSLRSEDTAVYFCARAGKRFGESYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 74 - protein, 705 aa, synthetic construct
DIQMTQSPSAMSASVGDRVTITCRASQGISNYLAWFQQKPGKVPEPLIYAASSLQSGVPSRFSGSGSVTEFTLTISSLQPEDFATYYCLQHNSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSEGKSSGSGSESKSTEGKSSGSGSESKSTGGGGSQVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGEAVGGNYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 75 - protein, 705 aa, synthetic construct
DIQMTQSPSAMSASVGDRVTITCRASQGISNYLVWFQQKPGKAPKRLIYAVSSLYSGVPSRFSGSGSGTEFTLTISSLQPEDFATYYCLQHDSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSEGKSSGSGSESKSTEGKSSGSGSESKSTGGGGSQVQLVQSGAEVKKPGASVKVSCKASGYTFTSYYVHWVRQAPGQGLEWMVIINPGGGTTSYAQKFLGRVTMTRDTSTNTVYMELKSLRSEDTAVYYCARGEAVTGNYFYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 76-78 - skipped (3 entries)

SEQ ID NO: 79 - protein, 707 aa, synthetic construct
EAVVTQEPSLTVSPGGTVTLTCRSSTGAVTTSNYANWVQEKPGQLPRGLIGGTNKRAPWVPARFSGSLLGGKAALTLSGAQPEDEAEYFCALWYSNLWVFGGGTKLTVLGQPKAAPSVTLFPPSSEELQANKATLVCLISDFYPGAVKVAWKADGSPVNTGVETTTPSKQSNNKYAASSYLSLTPEQWKSHRSYSCQVTHEGSTVEKTVAPAECSGGGGSEGKSSGSGSESKSTEGKSSGSGSESKSTGGGGSEVQLVESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 80 - protein, 707 aa, synthetic construct
EAVVTQEPSLTVSPGGTVTLTCRSSTGAVTTSNYANWVQEKPGQLPRGLIGGTNKRAPWVPARFSGSLLGGKAALTLSGAQPEDEAEYFCALWYSNLWVFGGGTKLTVLGQPKAAPSVTLFPPSSEELQANKATLVCLISDFYPGAVKVAWKADGSPVNTGVETTTPSKQSNNKYAASSYLSLTPEQWKSHRSYSCQVTHEGSTVEKTVAPAECSGGGGSEGKSSGSGSESKSTEGKSSGSGSESKSTGGGGSEVQLVESGGGLVQPGGSLKLSCAASGFTFNTYAMNWVRQAPGKGLEWVARIRSKYINYATYYADSVKDRFTISRDDSKNTAYLQMNNLKTEDTAVYYCVRHGNFGNSYVSWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 81 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 82 - protein, 226 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 83 - protein, 226 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 84 - protein, 228 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 85 - protein, 228 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 86 - protein, 228 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSLG

SEQ ID NO: 87 - protein, 107 aa, synthetic construct [sequence omitted]

SEQ ID NO: 88 - protein, 106 aa, synthetic construct
GQPKAAPSVTLFPPSSEELQANKATLVCLISDFYPGAVKVAWKADGSPVNTGVETTTPSKQSNNKYAASSYLSLTPEQWKSHRSYSCQVTHEGSTVEKTVAPAECS

SEQ ID NO: 89 - protein, 38 aa, synthetic construct
GGGGSEGKSSGSGSESKSTEGKSSGSGSESKSTGGGGS

SEQ ID NO: 90 - protein, 103 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC